(19) United States
(12) Patent Application Publication
(10) Pub. No.: US 2026/0242806 A1
(43) Pub. Date: Aug. 20, 2026

(54) CONTRACTILE INJECTION SYSTEM AND USE THEREOF

(71) Applicant: KØBENHAVNS UNIVERSITET, Copenhagen K (DK)

(72) Inventors: Eva Maria REBROVA, Copenhagen K (DK); Nicholas TAYLOR, Copenhagen K (DK)

(21) Appl. No.: 18/876,631

(22) PCT Filed: Jun. 30, 2023
(86) PCT No.: PCT/EP2023/068102

§ 371 Date: Dec. 18, 2024

(30) Foreign Application Priority Data
Jun. 30, 2022 (EP) 22182318.0

(51) Int. Cl. C12N 15/74; A01N 63/50; A01P 1/00; A01P 3/00; A01P 5/00; A01P 7/04; C07K 14/255; C07K 14/27; C12R 1/18; C12R 1/19; C12R 1/42; C12R 1/425

(52) U.S. Cl. CPC C12N 15/74; A01N 63/50; A01P 1/00; A01P 3/00; A01P 5/00; A01P 7/04; C07K 14/255; C07K 14/27; C07K 2319/02; C07K 2319/55; C12R 2001/18; C12R 2001/19; C12R 2001/42; C12R 2001/425

(57) ABSTRACT

The invention relates to re-engineering of a natural microbial syringe for loading of effector proteins that are not naturally found as cargo of the specific syringe. The cargo, that is one or more effector protein and proteins required for packing, can be up to 300 kDa and can be proteins such as toxins, therapeutic proteins, and gene modification tools. The re-engineering of the natural syringe result in a contractile injection system that allows packing of large cargo and cargo combinations, such as toxin-chimeras. The system comprises a Conserved Virulence Cassette encoding the syringe structure and is co-expressed with a sequence encoding cargo and a signal peptide. The signal peptide comprises a packing motif that enables packing of cargo into the syringe. The contractile injection system can be used for agricultural purposes as well as for medicinal purposes.

### FIELD

The present invention relates to a genetically modified Contractile Injection System (CIS) syringe. CIS's are natural cell-free proteinaceous nano-scale syringes that are prevalent in bacteria and archaea. The syringes can bind to specific target cells, such as bacteria, insect or plant cells, and inject effectors, such as toxins, into them. The CIS structure comprises a contractile sheath enveloping a rigid tube that is sharpened by a spike-shaped protein complex at its tip. The spike complex forms the centerpiece of a baseplate complex that terminates the sheath and the tube. The baseplate anchors the tail to the target cell membrane with the help of fibrous proteins emanating from it and triggers contraction of the sheath. The contracting sheath drives the tube with its spiky tip through the target cell membrane. Subsequently, the cargo is injected through the tube and into the target cell.

### BACKGROUND

Microorganisms exist in a competitive natural environment, resulting in the evolution of toxin injecting molecular machines. One such machine, the extracellular contractile injection systems (eCIS), are natural nano-scale syringes, that can bind to specific target cells, and inject effector toxins into them. One of the systems that has been investigated is the anti-feeding prophage (Afp) injection system, encoded on the natural pADAP plasmid in the bacterial species Serratia entomophila.

Many microorganisms have similar toxin-injecting syringes carrying different toxins. All of the systems are believed to be species-specific to their target. The bacterial variety and abundance of CIS syringes offer tremendous opportunities to be exploited for targeted delivery of molecules, effectors, and drugs.

Current state of the art investigates the genetic modification of Photorhabdus Virulence Cassettes (PVCs) needle tips, wherein the genetic modifications comprise truncation of the native PVC effector toxin down to its signal sequence of 50 amino acids, translocating a variety of heterologous effector toxins from other organisms or CRISPR/Cas enzymes within the needle complex via fusion with the truncated signal sequence, and modification of the tail fibre domain of the needle complex for species-specific targeting (WO02020245611A1). Other CIS systems that have been investigated are for example Metamorphosis Associated Contractile (MAC) syringes for the delivery of proteinaceous cargo/heterologous protein/peptide to a cell (WO2020102746A1) and Type III secretion systems disclosing fusion polypeptide methods, comprising a needle tip-translocator complex (or antigenic fragments thereof) and truncated bacterial effector peptides (WO2019217243A1, WO2020223601A1). Furthermore, nucleic acid expression vectors comprising an inhibitory Type III secretion system nucleic acid sequence, and further comprising a CIS acting regulatory element capable of driving transcription of the nucleic acid sequence in a plant cell to confer bacterial pathogen resistance to the cell, further wherein the dominant negative T3SS protein mediates assembly of a dysfunctional needle complex (WO2010117406A2).

Other related technologies available on market for agricultural pest management, are Bt (Bacillus thuringiensis) crops, manipulation crop strains by representing crystalline toxins that must be activated by alkaline lysis; predatory species, such as Adaline, Amblyline, Encarline, Erviline etc. by Bioline AgroSciences which are life formulations, predatory ladybug, mites, parasites, wasps; Photorhabdus luminescens toxin sequences, Eric Duchard et al., WO2003087377A9 WIPO (PCT), drawback in stability, targeted delivery, and off target problems; and life bacterial formulations e.g., Actinovate® by Novozymes A/S, which is a naturally occurring bacterium (Streptomyces lydicus WYEC 108) and bionematicidal formulations, AZAGUARD@, BIOCERES®, and BT NOW® by BioSafe Systems of entomopathogenic fungi and bacteria; to control agricultural pests such as nematodes, fungi, and insects.

Evolutionary related technologies are, Bacteriophage (BP) Therapies, which have a couple of limitations in terms of specific activity, difficulties in production of BP strains without antibiotic resistant genes or bacterial virulence factors, formulation and stability challenges, emerging resistance, and contribution to said resistance and reduced activity due to immune system responses to BPs. The anti-feeding prophage (Afp) is a CIS, encoded on the natural pADAP plasmid in the bacterial species Serratia entomophila. The purified Afp syringe, including a 264 kDa injected toxin, Afp18, causes a rapid anti-feeding effect against the New Zealand grass grub larvae, resulting in larval starvation and mortality (Hurst et al., 2007). The Afp CIS is proposed to be extracellular, meaning non-membrane bound and secreted for targeted delivery of toxins. The genomic CIS syringe operon cassette comprises a conserved syringe core building up a contractile sheath, tail tube, baseplate complex, central spike, an AAA ATPase proposed to be involved in syringe (sheath) assembly or packing of the syringe and C-terminally located effectors. The genomic organization and multiprotein assembly of the Contractile Injection System (CIS) from Serratia entomompila (Afp) is shown in FIG. 1. As shown in FIG. 1 (A), the genome cluster comprises 18 open reading frames (ORFs) as encoded on the natural plasmid pADAP and a Conserved Virulence Cassette (CVC) building up the syringe and C-terminal effectors/toxins associated with the syringe. The pADAP Afp gene cluster naturally consists of 18 Open Reading Frames (ORFs, SEQ. NO. 2-19). The ORFs afp1-16 (SEQ. NO. 2-17) build up the syringe. Downstream of afp16, sits a hypothetical remnant toxin afp17 (SEQ. NO. 18, 1077 nt/358 amino acids) and a large afp18 toxin (7101 nt/2366 amino acids) at the end of the cluster. FIG. 1 (B) is a negative-stain Electron Microscopy (EM) image of the Afp syringe, comprising a proteinaceous multi-protein assembly (around 10 MDa) building up an injection machinery of an average of 120 nm in length, 20 nm in width and syringe features highlighted (white arrows).

The structure of Afp and related CIS syringes have been published recently (Desfosses et al., 2019, Jiang et al., 2019, Xu et al., 2022, Jiang et al. 2022), however mechanism of action and in particular cargo packing, translocation mechanisms and maximum size of cargo are unknown.

The S. entomophila and related CIS syringes are extremely stable because of unique operon set up with stabilizing features, they are extracellular, have tail fiber legs for cell attachment, and can carry huge toxin proteins, when compared to competing technologies e.g., Type III secretion systems, which are membrane associated. Cell specific tail fibers stabilize the syringe and can be modified for targeted cell delivery. The Afp syringe can be produced in E. coli in high abundance with years of shelf life at fridge temperatures. The Afp syringe is stable in presence and absence of cargo, and in presence of toxin-chimeras or heterologous non-syringe related cargo modifications.

The use of these systems for targeted delivery of drugs is of high interest since those syringes transport translated proteins, instead of competing technologies that transport RNA or DNA (phage therapy) and are transporting cargo to specific eukaryotic or bacterial cells via tail fibers. Those syringes have been shown to be associated with a row and variety of effectors and in particular insect toxins, however, the mechanism of action has not been revealed to date and moreover the detrimental factor for allocation of various cargos is an obstacle.

Thus, a cell-free CIS system modified in such a way that different kinds of cargo can be introduced into various target cells in a controlled manner would be a beneficial tool in pest management as well as for medicinal purposes.

### SUMMARY

This invention relates to re-engineering of a natural microbial syringe for loading of effector proteins that are not naturally found as cargo of the specific syringe. The cargo, that is one or more effector protein and proteins required for packing, can be up to 300 kDa and can be proteins such as toxins, therapeutic proteins, and gene modification tools. The re-engineering of the natural syringe result in a contractile injection system that allows packing of large cargo and cargo combinations, such as toxin-chimeras. The system comprises a Conserved Virulence Cassette encoding the syringe structure and co-expression of a signal peptide fused to the sequence encoding the cargo.

The signal peptide comprises a packing motif that enables packing of cargo into the syringe. The contractile injection system can be used for agricultural purposes as well as for medicinal purposes.

In a first aspect, the present invention provides a contractile injection system comprising a Conserved Virulence Cassette controlled by a promoter, said Conserved Virulence cassette comprising structural operons selected from subtypes Ia, Ib, IIa, IIb, IIc, or IId, comprising Open Reading Frames (ORF) encoding a proteinaceous phage tail like syringe structure which functions as a delivery system of cargo, said Conserved Virulence Cassette being co-expressed with an Open Reading Frame under the control of a second promoter encoding a signal peptide and effector cargo, said signal peptide comprising a packing motif that enables packing of effector cargo into the syringe, said effector cargo being different from the effector cargo found in the natural contractile injection system of the Conserved Virulence Cassette and being of up to 300 kDa; characterized in that said packing motif has a covariance less than zero, said covariance being a function of a first vector and a second vector, said first vector comprising the amino acid hydrophilicities of each amino acid in said packing motif and said second vector comprising the electronic properties of each amino acid in said packing motif, said covariance having a lag of 2 for said first vector or for said second vector.

In a second aspect, the present invention provides for the use of a contractile injection system of the first aspect for controlling agricultural pests, such as bacteria, nematodes, fungi, and insects at any developmental stage.

In a third aspect, the present invention provides a contractile injection system of the first aspect comprising a medicament or a genetic modification tool as effector cargo for use in delivering a medicament or a genetic modification tool.

In a fourth aspect, the present invention provides a composition comprising a contractile injection system of the first aspect.

### BRIEF DESCRIPTION OF DRAWINGS

FIG. 1: (A) Schematic drawing of the genomic organization and multiprotein assembly of the CIS from Serratia entomophila, (B) Negative-stain Electron Microscopy image of the CIS from Serratia entomophila syringe.

FIG. 2: Schematic drawing of a recombinant co-expression protocol.

FIG. 3: Diagrams of properties of packing motifs (A) drawing showing the classification of amino acid properties with a focus on polar amino acids (B) diagram showing packing motif representation based on multiple sequence alignment highlighting high polar amino acid abundance (C) sequence alignment.

FIG. 4: Table showing CC values of predicted packing motifs of syringe cluster proteins.

FIG. 5: Diagram alignment of the polarity of amino acid signal peptides comprising packing motifs.

FIG. 6: (A) Negative staining electron micrographs of intact syringes, (B) SDS PAGE syringe resolution, (C) Immunodetection blot.

FIG. 7: (A) and (B) Negative staining electron micrographs of syringes at different temperatures.

FIG. 8: Schematic drawing of Afp18 N-terminal and C-terminal truncations (SEQ. NO. 115-149) and tables disclosing sizes of the constructs.

FIG. 9: (A) SDS PAGE Coomassie staining (B) and (C) Western blots.

FIG. 10: Negative staining electron microscopy of syringes.

FIG. 11: (A) Immunodetection blots of syringe and toxin-chimeras, (B) negative staining electron microscopy of syringes.

FIG. 12: (A) structure of Casphi2 (SEQ. NO. 99, 102, 105, 108, 111, 113, 152) (left), immunodetection blot (mid), and negative staining electron microscopy of syringes, (B) structure of LL37 (SEQ. NO. 82, 85, 88, 91, 94, 96, 161)(left), immunodetection blot (mid), and negative staining electron microscopy of syringes.

FIG. 13: Immunodetection blots of Afp18 truncation variants.

FIG. 14: SDS PAGE (left) and WESTERN Blot (right) of Afp syringe and N- and C-terminally truncated Afp18 toxin.

FIG. 15: Negative staining electron microscopy of syringes of Afp18 toxin N- and C-Terminal truncation variants.

FIG. 16: Negative staining electron microscopy of syringes of Afp18 Toxin N- and C-Terminal Truncation Variants.

FIG. 17: (A) Sequence, structure, and parameter comparisons of three signal peptides, (B) immunodetection with antibodies and negative staining electron microscopy of intact syringes, (C) immunodetection blot.

FIGS. 18 and 27: Table disclosing homology search of novel packing motifs and CIS syringes tested in Example 4 (A) and Table disclosing polar aa content and CC values of sequences tested in Example 4.

FIG. 19: Table disclosing packing motif parameters for N-terminal peptides of CIS cargo.

FIG. 20: N-terminal packing domains from other CIS species effectors pack non-CIS related Casphi2, into Afp. A) selection of N-terminal signal domains (see FIGS. 18A and 19) with high negative CC values was chosen to investigate whether they can pack non syringe related cargo into Afp. Signal domains were chosen from a variety of bacterial species e.g., Salmonella enterica and Photorhabdus luminescens Virulence Cassettes (PVCs). Co-production of Afp syringe and signal domain chimeras was carried out. (B) Signal domain properties highlighted using peptide calculator https://www.bachem.com/knowledge-center/peptide-calculator/. Hydrophilic amino acids are highlighted with green bars, hydrophobic with red. It is apparent that signal domains have high abundance of hydrophilic and low amount of hydrophobic amino acids. (C) Immuno-detection blotting of syringe and signal domain-Casphi2 chimeras. Antibodies against syringe (SEQ. NO. 202 or 203) and N-terminal signal domains, CyaANT20_ag (SEQ. NO. 285 binding to SEQ. NOs. 265-267), EPTox20_ag (SEQ. NO. 284 binding to SEQ. NOs. 266-268), SE_Afp18-ag1 (SEQ. NO. 200) and Casphi2 (commercially available monoclonal antibody Cas12j Antibody (MA5-47448) in WB, Thermo Fisher) were used to detect successful purification. (D) Mass spectrometry confirmation of co-production and presence of N-terminal domains and Casphi2. (E) Negative staining electron micrographs of syringes, validating syringe integrity and architecture.

FIG. 21: N-terminal packing domain of effectors from other CIS species that were shown not successful (not detectable) packing of Casphi2. (A) Selection of potential non-qualified N-terminal signal domains based on physico-chemical properties (highly positive CC value, hydrophobicity) from a variety of bacterial species e.g., Pseudomonas aeruginosa and Photorhabdus luminescens (PVCs). Co-production of Afp syringe and signal domain chimeras was carried out. (B) Signal domain properties highlighted using peptide calculator https://www.bachem.com/knowledge-center/peptide-calculator/. Hydrophilic amino acids are highlighted with green bars, hydrophobic with red. Higher amount of hydrophobic amino acids an be observed and lower hydrophilicity. (C) Immuno-detection blotting of syringe and signal domain-Casphi2 chimeras. Antibodies against syringe and N-terminal signal domains and Casphi2 (commercially available monoclonal antibody Cas12j Antibody (MA5-47448) in WB, Thermo Fisher), ExoUNT20_ag (SEQ. NO. 286 binding to SEQ. NOs. 272-274), SEAfp17NT20_ag (SEQ. NO. 288 binding to SEQ. NO. 269-271) were used to detect successful purification. (D) Negative staining electron micrographs of syringes, validating syringe integrity and architecture.

FIG. 22: Mutational analysis of native SEAfp18N20 packing domain. (A) Mutational analysis of Afp's native packing domain SEAfp18NT20 (SEQ. NO. 103). Hydrophilic residues are replaced by non-polar alanine. (B) Signal domain properties highlighted using peptide calculator https://www.bachem.com/knowledge-center/peptide-calculator/. Hydrophilic amino acids are highlighted with green bars, hydrophobic with red. Decreasing amount of hydrophilic amino acids down to 50% and replacing highly hydrophilic residues, lysines, threonines and glutamic acids (K, T, E) by nonpolar amino acid alanine. (C) Immuno-detection blotting of syringe and signal domain mutants-Casphi2 chimeras. Antibodies against syringe (SEQ. NO. 202/203) and N-terminal signal domains SE_Afp18-ag1 (SEQ. NO. 200), SEAfp18NT20KtA_ag (SEQ. NO. 288) and Casphi2 (commercially available monoclonal antibody Cas12j Antibody (MA5-47448) in WB, Thermo Fisher), were used to detect successful purification. Mass spectrometry validation is included in ‘FIG. 20: N-terminal packing domains from other CIS species effectors pack non-CIS related Casphi2, into Afp. (D)’. (D) Negative staining electron micrographs of syringes, validating syringe integrity and architecture.

FIG. 23: Cryo-EM analysis of empty and syringes with Afp18 toxin and toxin chimeras. (A) From left to right: Full length Afp (SE1-18, red, SEQ. NO.1: pBAD33_SE1-18) shows small density along the lower third of the tail tube, Afp1-18ΔC4 (SE1-18C4, dark red, SEQ. NO.20: pBAD33_SE1-18C4), Afp with half the Afp18 toxin size present shows diminished density inside the tube but remaining cargo close to the baseplate, SE1-17 (grey, SEQ. NO. 39: pBAD33_SE1-17) with small density close to the baseplate and tip entry (potentially Afp6 helices), SE1-16 (blue, SEQ. NO.57: pBAD33_SE1-16) the Afp particle without any cargo shows no density in the inner tube, SE1-16+18C8-Casphi2 (brown, SEQ. NO. 57: pBAD33_SE1-16 and SEQ. NO.150:pET11a_Afp18C8-Casphi2), the Afp particle with Afp18C8 fused to Casphi2 shows novel partially structured density appearing all along the tail tube. (B) High-resolution cryo-EM maps of two non-eCIS cargos loaded inside the Afp tail tube, Afp18C8 fused to Casphi2 (brown, SEQ. NO. 57: pBAD33_SE1-16 and SEQ. NO.150: pET11a_Afp18C8-Casphi2) and to ExoU (purple, SEQ. NO. 57: pBAD33_SE1-16 and SEQ. NO.153:pET11a_Afp18C8-PAExoU). The tail tube is filled at various locations along the tube and partially structured elements can be observed. Syringe components were validated ahead of cryo-EM analysis (see FIG. 25 Validation of stability and protein entities of Afp18 toxin-effector chimera preparations.)

FIG. 24: In vivo efficacy of native Afp and modified Afp syringes in G. melonella larvae. (A) Natively expressed Afp (SE1-18), Afp without Afp18 (SE1-17), Afp without any toxin (SE1-16), empty pBAD33 vector expression and buffer conditions (1×PBS) were injected into G. melonella larvae. SE1-18 causes larval mortality between 5-13 days. SE1-16 induces developmental arrest (dormancy). (B) Afp revealing high temperature stability (Tstabil) with observed temperature induced ejection between 58-60° C. Particles exposed to respective temperatures for 10 min in 1×PBS and analyzed over negative staining electron microscopy. (C) Anti-eukaryotic effectors fused to Afp18 N-termini were investigated for their efficacy in vivo, and co-produced with SE1-16 syringes. The syringe preparations were injected and increased mortality after 3 days can be observed for ExoU and LL37 effectors.

FIG. 25: Validation of stability and protein entities of syringe and Afp18 toxin-effector chimera preparations. (A) Architecture of SE1-16 syringes co-produced with Afp18C8-effectors. All preparations show complete syringe architecture and similar production levels. (B) Temperature stability of preparations investigated at 50° C. or 10 min in 1×PBS and analyzed using negative staining electron microscopy. Syringes appear stable after high temperature exposure. (C) Mass spectrometry validation of effector and syringe components being present. All expected proteins could be confirmed. (D) Coomassie protein gel analysis and immune-detection blotting confirming protein component of syringe and effectors being present. The syringe preparations were used for cryo-EM analysis (FIG. 23, Cryo-EM analysis of empty and syringes with Afp18 toxin and toxin chimeras)

FIG. 26 Table showing CC values of predicted packing motifs of syringe cluster proteins.

FIG. 27: Table disclosing polar aa content and CC values of sequences tested in Example 4.

### DETAILED DESCRIPTION OF THE INVENTION

The present invention relates to a re-engineered CIS syringe, for loading of effector cargo, such as insecticidal toxins, that differ in bacterial origin, toxin size and target activity. The re-engineered syringe allows packing of toxin combinations, termed toxin-chimeras. The identification and use of CIS packing motif allows syringe and non-syringe related effector cargo to be attached to the syringe. We have found that removal of one native Afp gene within the cluster (remnant toxin afp17), resulted in the possibility for packing of larger toxins. The syringe can be rapidly adapted to attach various toxin proteins and effector cargo for targeted transport, to specifically target pests, including resistant insect, worm, and nematode populations. This invention provides a customizable tool, one that can be developed and used as a future crop pesticide within the agricultural pest management sector, in the form of an insecticidal spray, providing a sustainable, easily adaptable, reproducible, and scalable pesticide for agricultural industry.

The exact genetic CIS modification of the syringe complex and toxins to allow attachment of heterologous effector toxins and non-syringe related effectors was hitherto unknown. We have now surprisingly found that the use of a minimum operon set up to produce a syringe, such as the Afp syringe, in combination with a previously unidentified minimum N-terminal packing motif, such as the packing motif of Afp18, to be attached to effector cargos of choice, such as toxins and gene modification tools, can transport cargo fusions. Moreover, we show herein that CIS related cargo that doesn't meet the herein identified packing motif characteristics cannot associate to the syringe. With the CIS according to the present invention, heterologous cargo and toxins in different sizes and target activity can be attached to the syringe by N-terminal attachment of the packing motif to effector cargo of interest.

The CIS of the present invention is based on the identification of a crucial packing motif of 20 amino acids at the N-terminus of Afp18 (SEQ. NO. 115-123 and 143-149), that allows attachment of CIS toxins, toxin-chimeras, and non-syringe related effectors with antibacterial, antifungal, and antiviral activity to the syringe to act as toxin and effector delivery tool. The herein identified packing motif has a high abundance of polar amino acids and has been found in a row of other CIS syringe cargo at the N-terminal region with high cross covariance and high abundance of polar amino acids.

The genetic modification of the Afp CIS system and related syringes described herein represents a non-life formulation, loaded with active translated toxins/effectors, that can be delivered to target cells without off-target activity. The system is easily adaptable, reproducible, offers economical long-time storage and is scalable for agricultural industry, for medicinal purposes, and for gene modification purposes.

The present disclosure provides a contractile injection system comprising a Conserved Virulence Cassette controlled by a promoter, said Conserved Virulence cassette comprising structural operons selected from subtypes Ia, Ib, IIa, IIb, IIc, or IId, comprising Open Reading Frames encoding a proteinaceous phage tail like syringe structure which functions as a delivery system of cargo, said Conserved Virulence Cassette being co-expressed with an Open Reading Frame under the control of a second promoter encoding a signal peptide comprising a packing motif that enables packing of effector cargo into the syringe, said effector cargo being different from the effector cargo found in the natural contractile injection system of the Conserved Virulence Cassette and being of up to 300 kDa; characterized in that said packing motif has a covariance less than zero, said covariance being a function of a first vector and a second vector, said first vector comprising the amino acid hydrophilicities of each amino acid in said packing motif and said second vector comprising the electronic properties of each amino acid in said packing motif, said covariance having a lag of 2 for said first vector and for said second vector.

### Definitions

Contractile injections system (CIS) means, in the context of the present invention, proteinaceous macromolecular machines produced by bacteria that are evolutionarily related to bacteriophage tails and are specialized to puncture membranes to deliver molecules.

Conserved Virulence Cassette (CVC) means, in the context of the present invention, evolutionary conserved virulence genes encoded CIS on a genomic region in form of a structural operon.

Structural operon means, in the context of the present invention, a set of adjacent structural genes arranged under a common promoter and regulated by common operators for translation.

Class I and Class II CIS means, in the context of the present invention, phylogenetic lineage classification of CISs/CVCs into class I and class II, where class I has two and class II has four subtypes, subtype Ia and Ib and subtype IIa-d, respectively (Xu et al., 2022).

Open Reading Frame means, in the context of the present invention, a span of DNA sequence between a start and stop codon encoding for a protein.

Syringe Structure means, in the context of the present invention, bacterially produced multiprotein assemblies that form a syringe-like structure able to carry and transfer proteins/DNA/RNA/effectors through piercing and injection of membranes carried out by a contraction mechanism.

Cargo means, in the context of the present invention, any natural, putative, or synthetic nucleotide or protein that can be attached to a CIS. Effector cargo refers to the part of the cargo, such as a toxin, that provides an effect on the target.

Co-expressed means, in the context of the present invention, that different ORFs are expressed simultaneously. The different ORFs can be situated in order on the same plasmid or the different ORFs can be situated on different plasmids.

Signal Peptide means, in the context of the present invention, a peptide which is fused naturally or synthetically to facilitate protein translocation.

Packing Motif means, in the context of the present invention, a specific area of a signal peptide with shared/conserved structural, functional, biophysical, or biochemical peptide characteristics crucial for functional translocation.

Fused to means, in the context of the present invention, joined or blended to form a single entity. Fusing/joining the packing motif to natural, putative, or synthetic nucleotides or proteins on genomic or protein level means that it can be done in any known way such as via cloning, protein-protein interaction, or cross-linking.

Hydrophilicity means, in the context of the present invention, physiochemical property of amino acids that are of polar nature and therefore are of, relating to or having a strong affinity for water.

Electronic Properties means, in the context of the present invention, a description of polarity and charge of an amino acid or an amino acid chain.

Lag refers to, in the context of the present invention, how far a series is offset. For instance, a lag of 1 autocorrelation is the correlation between values that are one unit apart. In auto- and cross-correlation and covariance (ACC) transforms a maximum lag describes the longest sequence stretch used. A lag of 1 if parameters account only for the nearest neighbor interaction, same transformation applied to the next-near-neighbor represents a lag of 2, etc. First and second vector means, in the context of the present invention, a mathematical vector used to describe hydrophilicities of each amino acid in said packing motif or electronic properties of each amino acid in said packing motif, respectively.

Expression vector means, in the context of the present invention, an expression vector, otherwise known as an expression construct, is a plasmid or virus designed for gene expression of nucleic acid in cells. The vector is used to introduce a specific gene into a target cell, and can commandeer the cell's mechanism for protein synthesis to produce the protein encoded by the gene. Expression vectors are the basic tools in biotechnology for the production of proteins. The vector may be engineered to contain regulatory sequences that act as enhancer and promoter regions and lead to efficient transcription of the gene carried on the expression vector. Expression vector may be used interchangably with vector in the present invention.

Cross Covariance means, in the context of the present invention, transformations to describe changes in physicochemical properties and property combinations over sequence stretches of different lengths.

Correlation means, in the context of the present invention, how much increase/decrease in one variable causes the other variable to increase/decrease and vice-versa.

Covariance means, in the context of the present invention, a measure of the joint variability (variance) of two variables.

The contractile injection systems of the present invention can be extracellular, or membrane bound depending on the CVC applied. Numerous CIS have been identified and they are phylogenetically diverse and comprises a superfamily of broadly distributed putative CIS loci. A dedicated online open-access database named dbeCIS discloses an analysis of the putative CIS loci (http.//www.mgc.ac.cn/dbeCIS/) and a CVC in the contractile system of the present invention can for instance be selected and identified in this database. The database covers both Gram-negative and Gram-positive bacteria, as well as archaea. The CVC loci are sub-divided into six specific subfamilies with distinct patterns in terms of genetic composition and organization. Further comparative analysis of the predicted CIS in the database with potential CIS can be used to identify other CVC that can be applied in the system presented herein.

There are two classes of CVCs that can be applied in the present CIS, Class I and Class II. Class I comprises two subtypes Ia and Ib. Members of class I are from gram negative bacterial loci only where, Ia derived from γ and β Proteobacteria, subtype Ib comprises loci not only from the Proteobacteria but also from other Gram-negative phyla including Bacteroidetes and Cyanobacteria. Class II comprises four subtypes derived from Gram-negative and Gram-positive bacterial and archaeal genomes, where subtype IIa-IId show strong correlation to Class I, however with Class II members in addition to the eight (ORF1/5, 2/3/4, 7, 8, 9, 11, 15, 16) core backbone subunits of CISs, also comprises subtype-specific arrangements and several additional subtype-specific genes classified based on comprising distinct patterns in terms of genetic composition and organization of the core components.

The Open Reading Frames class I or class II, of Open Reading Frames encoding a proteinaceous phage tail like syringe structure which functions as a delivery system of cargo, can been subdivided into two (Ia, Ib) and four (IIa-IId) subtypes, members of subtype I, from gram negative bacterial loci only where, Ia derived from γ and β Proteobacteria, subtype Ib comprises loci not only from the Proteobacteria but also from other Gram-negative phyla including Bacteroidetes and Cyanobacteria; members of subtype II containing loci derived from Gram-negative and Gram-positive bacterial and archaeal genomes, where subtype IIa-IId showing strong correlation to subtype I, however with type II members in addition to the eight (ORF1/5, 2/3/4, 7, 8, 9, 11, 15, 16) core backbone subunits of CISs also have subtype-specific arrangements and several additional subtype-specific genes classified based on distinct patterns in terms of genetic composition and organization of the core components.

In one preferred embodiment, the Conserved Virulence Cassette comprising structural operons selected from subtypes Ia, Ib, IIa, IIb, IIc, or IId, comprises up to 16 Open Reading Frames encoding a proteinaceous phage tail like syringe structure which functions as a delivery system of cargo

In another preferred embodiment, the Conserved Virulence Cassette comprising structural operons selected from subtypes Ia, Ib, IIa, IIb, IIc, or IId, comprises at least 16 Open Reading Frames encoding a proteinaceous phage tail like syringe structure which functions as a delivery system of cargo

In a preferred embodiment, the Conserved Virulence Cassette is of Class 1 and selected from the group comprising subtypes Ia and Ib derived from Gram-negative phyla from Proteobacteria, Bacteroidetes and Cyanobacteria.

In another preferred embodiment, the Conserved Virulence Cassette is an anti-feeding prophage cassette of subtype Ia with Open Reading Frames afp1 to afp16, from bacteria of the genus Serratia, Salmonella, Erwinia, Yersinia, or Photorhabdus.

In another preferred embodiment, the Conserved Virulence Cassette is selected from gene locations: afp1 (AAT48338, such as KHA73_24215)-afp18 (AAT48355, such as KHA73_24130) from Serratia entomophila, DJ39_RS03165-DJ39_RS03245 from Yersinia ruckeri, L581_RS22600-L581_RS22520 from Serratia fonticola, IFT93_RS22375-IFT93_RS22455 from Erwinia persicina, AEP37_RS09525-AEP37_RS09605 from Yersinia pekkanenii, JFQ86_16445-JFQ86_RS16365 from Serraia ureilytica, C0558_08345-C0558_08275 from Serratia marcescens, JDD69_004072-JDD69_004088 from Salmonella enterica, PluDJC_08620-PluDJC_08545, PluDJC_08725-PluDJC_08650, PluDJC_08815-PluDJC_08740, PluDJC_08925-PluDJC_08845, PluDJC_12605-PluDJC_12675, PluDJC_13295-PluDJC_13220 from Photorhabdus luminescens, and PAU_RS10200-PAU_RS10140, PAU_RS10855-PAU_RS10770, PAU_RS09635-PAU_RS09710, PAU_RS13565-PAU_RS13640, PAU_RS16655-PAU_RS16580 from Photorhabdus asymbiotica.

The promoter controlling the CVC can be the native CVC promoter or another promoter. The promoter can be an inducible promoter or a constitutively expressed promoter. Preferably, the promoter controlling the CVC is constitutively expressed.

The second Open Reading Frame in the CIS encodes a signal peptide comprising a packing motif that enables packing of effector cargo into the syringe and the effector cargo. This ORF is under the control of a second promoter that can be a native CIS promoter or another promoter. The second promoter can be the same as the promoter controlling the CVC.

Any commercially available promotor control systems suitable for bacterial, insect and mammalian protein expression are suitable for expression of the CVC of the present CIS and/or for controlling the ORF that encodes a signal peptide comprising a packing motif that enables packing of cargo into the syringe and the cargo.:

Preferably, the promoter controlling the CVC is a controlled or constitutively expressed promoter regulation system. Preferably, the promoter controlling the signal peptide and effector cargo is an inducible promoter.

Examples of suitable constitutive promotor systems is genome-integrated T7 expression systems, such as σ70 promoters and lacIQ promoters.

Examples of suitable controlled expression in bacteria comprises chemically inducible promoters such as Tac promoters (lactose/IPTG inducible), T7 LacO promotors (lactose/IPTG inducible), pBAD promotors (arabinose inducible, glucose repressible), pTrc, hybrid of trp and lac promoters (Lactose/IPTG inducible), OR2-OR1-PR, pLtetO, pLlacO (Anhydrotetracycline, lactose, IPTG), alcohol-inducible AlcA promoters, Steroid-regulated LexA promoter), pTetO (Anhydrotetracycline); temperature inducible promoters such as Hsp70 and Hsp90-derived promoters; light-inducible promoters such as Opto-Cre-Vvd—blue light inducible system, eLightOn system, FixK2, pR_FixK2 (Blue Light (470 nm), Red light inducible inverter (pCph8 and pPLPCB(S), that makes lacZ expression inducible by red light), PcpcG2(Green Light (532 nm)).

In a preferred embodiment, the promoter controlling the CVC is the native CVC promoter and the promoter controlling the signal peptide and cargo is an arabinose inducible promoter.

In order for the system to be complete, the CVC and the signal peptide and cargo must be co-expressed meaning that these ORFs must be expressed at the same time/simultaneously. Co-expression can be provided by different means. The different ORFs can be situated on the same plasmid or on different plasmids.

The system tested in the examples herein was made by using a recombinant co-expression protocol, which allows rapid cloning and attachment of genes encoding effector proteins (FIG. 2). As shown in FIG. 2, The Afp syringe (top) without cargo was expressed on an arabinose inducible nucleic acid expression vector (SEQ. NO. 57). Toxins and toxin-chimeras (bottom) were cloned into an IPTG inducible pET11a vector for co-expression and for toxin regulation (SEQ. NO. 74-112, 150-197). Afp18 N-termini (Afp18C4/C6/C8/C10, black, SEQ. NO. 120, 122, 142, 146) were fused with insecticidal toxins from homologous CVCs from Y. ruckeri, P. luminescens and P. asymbiotica, and effectors from a Type III and Type VI Secretion System, resulting in a variety of toxin-toxin and toxin-effector combinations of different sizes (41-239 kDa) (SEQ. NO. 153, 156, 165, 169, 191, 194, 197). The homolog to Afp18 in Y. ruckeri, Afp17 (237 kDa), and the Photopexin of P. luminescens (38.6 kDa) were co-purified without Afp18 fragments (SEQ. NO. 76, 78).

In that co-expression protocol, the ORF encoding the CVC is expressed by one plasmid (an arabinose inducible pBAD33 vector) whereas the ORFs encoding the signal peptide and effector chimeras, are expressed by another plasmid (IPTG induction on a pET11a vector). Thus, whereas it is a possibility to have all ORFs on one plasmid, the sequences required for obtaining a complete functional CIS need not be on the same plasmid as long as co-expression is provided.

It was found here that the cargo comprising the effector cargo and the protein(s) required for packing of cargo into the syringe of the provided CIS could be up to approximately 300 kDa in order for the cargo to be successfully packed into the syringe.

Various cargo sizes were tested and found to be successfully packed into the syringe. The cargo ranged from 6.8 kDa-290.8 kDa cf. Table 2. In Thus, cargo that can be successfully packed into the CIS of the present invention is of a size ranging from approximately 6 kDa-300 kDa, such as, such as 80-300 kDa, such as 100-300 kDa, such as 145-300 kDa, such as 190-300 kDa.

In a preferred embodiment, the cargo is large cargo, such as cargo of at least 150 kDa, such as cargo between 190-300 kDa. In Table 2, the large cargo that was successfully packed has a size from 196.4 kDa up to 290.8 kDa.

The effector cargo can be effector proteins from natural CISs other than the CIS wherefrom the CVC is obtained. Moreover, effector cargo can be effector proteins that are not naturally associated with CIS. We have found that the origin of total cargo up to 300 kDa is not limiting to the suitability as cargo in the present CIS provided that the signal peptide comprises a packing motif that meets the defined criteria explained below.

The sequence encoding cargo may encode an antiviral compound, an antibacterial compound, an antifungal compound, an anti-eukaryotic compound, or a gene modification tool.

In a preferred embodiment, the sequence encoding cargo is selected from genes encoding a phage related gene modification enzyme Casφ-2, a CRISPR/Cas gene or genes encoding insecticidal toxins.

It has so far not been possible to provide a CIS that is easily customized to a certain desired use because the requirements for packing any desired cargo into the existing syringes was unknown. The present examples reveal that in order for different kind of cargo to be packed into a syringe, a signal sequence comprising a certain packing motif with must be comprised by the CIS.

Whereas signal sequences can be long, it is shown here that signal sequences down to 20 amino acids are suitable as long as the packing motif is comprised by the 20 amino acid signal peptide (SEQ. NO. 81, 84, 87, 90, 93, 98, 101, 104, 107, 110, 115-123, 143-149).

In a preferred embodiment, the signal peptide comprising the packing motif consists of 20 amino acids.

Whereas the specific amino acid sequence of the signal peptide and of the packing motif can vary, we have found that the packing motif can be identified because it is characterized in having a covariance less than zero, when the covariance is a function of a first vector and a second vector, where the first vector comprises the amino acid hydrophilicities of each amino acid in the packing motif and wherein the second vector comprises the electronic properties of each amino acid in the packing motif, when the lag is of 2 for each vector.

The electronic properties are a measure of the molecules charge and polarity.

The covariance is in a preferred embodiment the cross-covariance, calculated in accordance with the following equation I:

$${CC}_{za \neq zb , lag} = {\sum }_{i}^{n - lag} \frac{Vza , i \times Vzb , i + lag}{{( n - lag )}^{p}} ,$$

wherein CC is the cross covariances between the first vector za comprising amino acid hydrophilicities of each amino acid in said packing motif and the second vector zb comprising the electronic properties of each amino acid in said packing motif, i is the position of each amino acid and is a number between 1 and 20, n=20 is the number of amino acids comprised by the vector, I=2 is the lag, p is the normalization degree and V is the descriptor value.

The descriptor value V represents the principal amino acid descriptors measured and calculated from the physico-chemical properties.

In a preferred embodiment, the principal amino acid descriptors as disclosed in Hellberg et al., 1987, is applied for the calculation.

Table 1 below discloses the calculated physico-chemical properties of 20 amino acids and values and ranges from −5.36 to +4.13 obtained from Hellberg et al., 1987:

TABLE 1
Descriptor Scales Z1, Z2 and Z3 for Amino acids.

| Amino acid | Z1 | Z2 | Z3 |
|---|---|---|---|
| Ala (A) | 0.07 | −1.73 | 0.09 |
| Val (V) | −2.69 | −2.53 | −1.29 |
| Leu (L | −4.19 | −1.03 | −0.98 |
| Ile (I) | −4.44 | −1.68 | −1.03 |
| Pro (P) | −1.22 | 0.88 | 2.23 |
| Phe (F) | −4.92 | 1.30 | 0.45 |
| Trp (W) | −4.75 | 3.65 | 0.85 |
| Met(M) | −2.49 | −0.27 | −0.41 |
| Lys (K) | 2.84 | 1.41 | −3.14 |
| Arg (R) | 2.88 | 2.52 | −3.44 |
| His (H) | 2.41 | 1.74 | 1.11 |
| Gly (G) | 2.23 | −5.36 | 0.30 |
| Ser (S) | 1.96 | −1.63 | 0.57 |
| Thr (T) | 0.92 | −2.09 | −1.40 |
| Cys (C) | 0.71 | −0.97 | 4.13 |
| Tyr (Y) | −1.39 | 2.32 | 0.01 |
| Asn (N) | 3.22 | 1.45 | 0.84 |
| Gln (Q) | 2.18 | 0.53 | −1.14 |
| Asp (D) | 3.64 | 1.13 | 2.36 |
| Glu (E) | 3.08 | 0.39 | −0.07 |

Hellberg refers to these “principal properties” of the amino acids and tentatively interpret them as related to hydrophilicity (z1), bulk (z2), and electronic properties (z3). For the purpose of determining the covariance of the packaging motif, the Z-scale for hydrophilicity (Z1) and the Z-scale for electronic properties (Z3) are applied.

Qualification as packing motif is thus done via an alignment-independent approach, using cross covariance (CC) which is a transformation into uniform vectors of principal amino acid properties described in z scales. The CC value can be found by use of common available computer programs, such as the VaxiJen server (http://www.ddgpharmfac.net/vaxijen/VaxiJen/VaxiJen.html) or other servers or programs that offer CC calculations of proteins. The VaxiJen server and most other programs and servers for CC calculations of proteins applies the z-scales disclosed in Hellberg for the calculations.

Thus, it is possible to determine whether a specific signal peptide comprises a packing motif that will enable packing of cargo of up to 300 kDa into the syringe by adding the amino acid sequence of the signal peptide into this server and performing the CC calculations.

If the calculated covariance, such as the CC value, is negative, i.e., less than zero, our data have confirmed that the tested signal peptide comprises a packing motif that will enable packing of cargo of up to 300 kDa into the syringe.

We have moreover found that packing motifs as identified herein by having a covariance less than zero comprises an overrepresentation of polar amino acids (negative polar and positive). For confirmed working packing motifs percentage of polar amino acids was found to be at more than 60% over 20 amino acids.

The polar amino acids can be interchanging positive and negative polar amino acids. In one embodiment, the positive polar amino acids are selected from lysine (K), histidine (H), arginine (R), and the negative polar amino acids are selected from glutamic acid (E), (D) supported by other polar amino acids asparagine (N), serine (S), threonine (T), Glutamine (Q) in optimized sequence distribution to achieve high negative CC1,3 (lag=2) values.

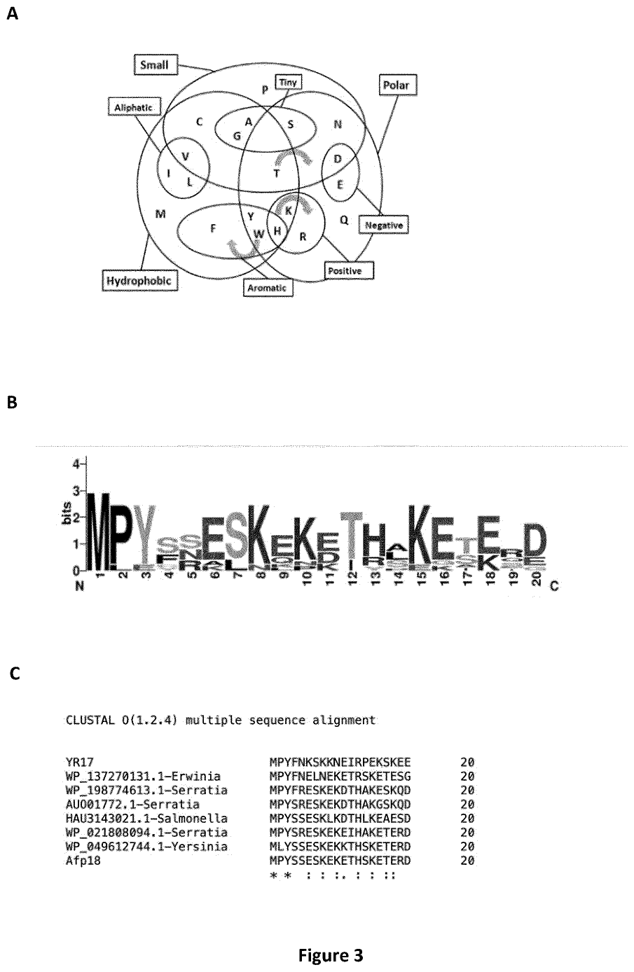

FIG. 3 (A) and (B) is a representation of packing motif sequence alignments using (http://weblogo.berkeley.edu). In FIG. 3 (B) the overall height of the stack indicates the sequence conservation at that position, while the height of symbols within the stack indicates the relative frequency of each amino or nucleic acid at that position. Polar residues (K), (R), (E), (D), (S), (T), (H) are overrepresented.

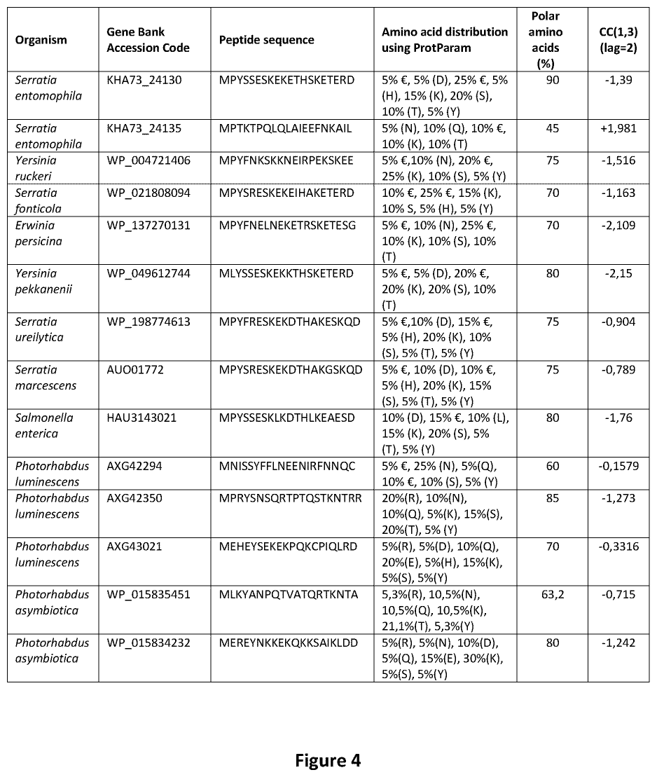

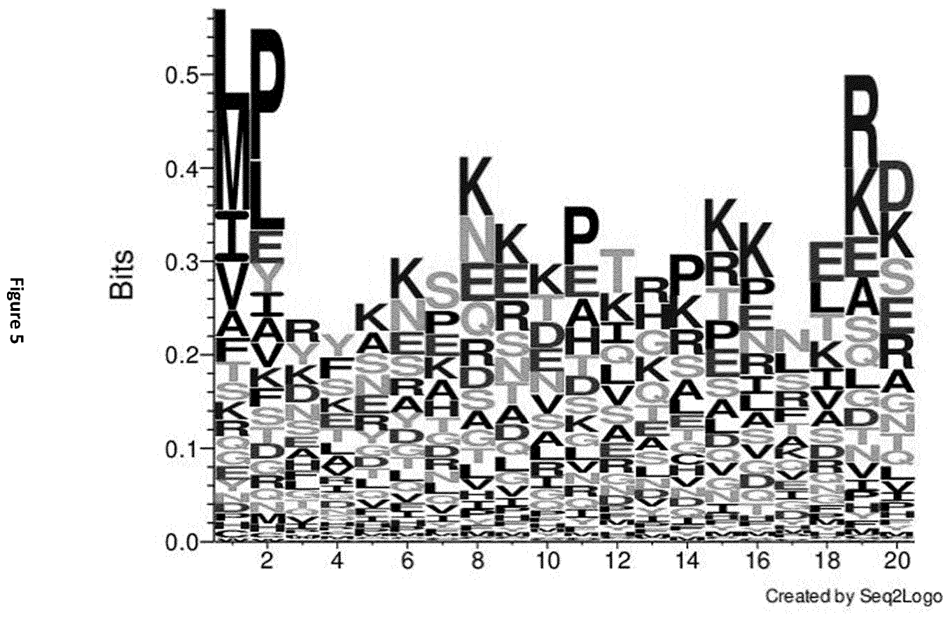

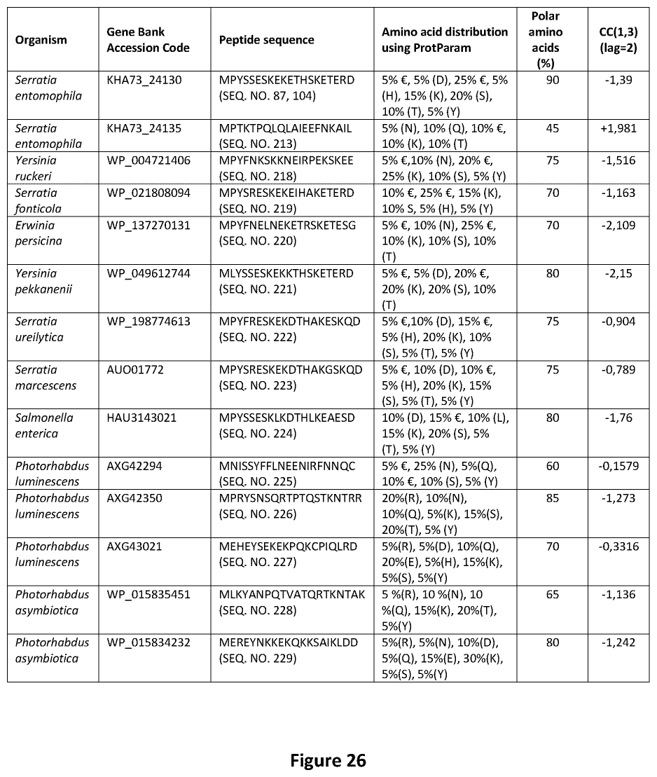

FIGS. 4 and 26 discloses a table comprising the polar amino acid content of known signal peptides from known CVCs, wherein the packing motif has a CC of below zero. The packing motifs are from evolutionary related syringes and the percentages of polar amino acids calculated over 20 amino acids using protparam webtool (https://web.expasy.org/protparam/) is included in the table. Negative CC(1,3)(lag=2) values can be used to identify any peptide within a sequence, that can serve as potential packing motif. As shown in FIGS. 4 and 26 out of 51 predicted syringe cluster proteins, that could be potentially packed, 35 were identified to have positive CC1,3(lag=2) values. FIGS. 4 and 26 comprises evolutionary related syringes and potential protein cargos within gene clusters. N-terminal sequences were investigated for qualification as packing motif using CC calculations cut off values (CC1,3 with lag=2).

FIG. 5 is a representation of evolutionary related packing motifs and amino acid preferences/motifs using Seq2Logo, a web-based sequence logo generation method for construction and visualization of amino acid motifs (https://services.healthtech.dtu.dk/service.php?Seq2Logo-2.0). It can be seen that polar amino acids play an important role (lysine (K), glutamic acid (E), asparagine (N), arginine (R).

In a preferred embodiment, the packing motif accordingly comprises more than 60% of polar amino acids selected from lysine, histidine, arginine, glutamic acid, asparagine, serine, threonine, glutamine, and combinations thereof.

In a preferred embodiment, the signal peptide in the contractile injection system according to the present invention is selected from:

(SEQ. NO. 87, 104)
MPYSSESKEKETHSKETERD,
(SEQ. NO. 218)
MPYFNKSKKNEIRPEKSKEE,
(SEQ. NO. 219)
MPYSRESKEKEIHAKETERD,
(SEQ. NO. 220)
MPYFNELNEKETRSKETESG,
(SEQ. NO. 221)
MLYSSESKEKKTHSKETERD,
(SEQ. NO. 222)
MPYFRESKEKDTHAKESKQD,
(SEQ. NO. 223)
MPYSRESKEKDTHAKGSKQD,
(SEQ. NO. 237)
MPYCNASKQKETYLKEIERD,
(SEQ. NO. 224)
MPYSSESKLKDTHLKEAESD,
(SEQ. NO. 205)
MPYTHSKETERDSSESKEKE,
(SEQ. NO. 206)
MPYDRETEKSHTEKEKSESS,
(SEQ. NO. 207)
MDPRYESTSEEKSSEHETKE,
(SEQ. NO. 208)
MRDPYTESSKEESHSKETKE,
(SEQ. NO. 209)
MPYSSESKEKETHSKE,
(SEQ. NO. 210)
MPYSSESKEKETHS,
(SEQ. NO. 211)
MPYSSESKEKET,
(SEQ. NO. 216)
MLPITAKKTNPFQELEQILA,
(SEQ. NO. 217)
MILPTKAKTFNPQEELQILA,
(SEQ. NO. 238)
MNISSYFFLNEENIKFNNQY,
(SEQ. NO. 230)
MLSTEKHNKDTKHPRNREKK,
(SEQ. NO. 231)
MPNSKYSEKVNHSANGAEKC,
(SEQ. NO. 226)
MPRYSNSQRTPTQSTKNTRR,
(SEQ. NO. 253)
MEHEYSEKEKPQGKKPLIKS,
(SEQ. NO. 234)
MEREYSEKEKHKKHPIQLRD,
(SEQ. NO. 235)
MVHEYSINDRQKRHSFSSAN,
(SEQ. NO. 228)
MLKYANPQTVATQRTKNTA,
(SEQ. NO. 239)
LLTHINLIFRVKCKYSICCLF,
(SEQ. NO. 240)
LCAFPIDGYTNERANQGCGE,
(SEQ. NO. 225)
MNISSYFFLNEENIRFNNQC,
(SEQ. NO. 241)
IIFTRDRNPTLSAHIKGGKK,
(SEQ. NO. 230)
MLSTEKHNKDTKHPRNREKK,
(SEQ. NO. 242)
EVNNGGNKSKAQAHTPDLVM,
(SEQ. NO. 243)
ELKHDDSKIKSQVSIPNLVK,
(SEQ. NO. 244)
TIVNPYSIYLKHIPNGFQDA,
(SEQ. NO. 227)
MEHEYSEKEKPQKCPIQLRD,
(SEQ. NO. 245)
LGDDIMPISNLAKESEVRAV,
(SEQ. NO. 232)
MYDSKKKNSEPTTKKKFERS,
(SEQ. NO. 246)
VGNKNTPSRVKIFISALIFM,
(SEQ. NO. 233)
MPNKKYSENTHQGKKPLIKS,
(SEQ. NO. 238)
MNISSYFFLNEENIKFNNQY,
(SEQ. NO. 247)
MPRYANYQJNPKQNIKNSHG,
(SEQ. NO. 248)
MMFDENLECVNEIINEKLD,
(SEQ. NO. 249)
SENKTKHNQQDSTAKDCWHE,
(SEQ. NO. 250)
ESHSDDKHRDQETNQKTANK,
(SEQ. NO. 251)
EDDSKDHFKQSRNQTEKHYN,
(SEQ. NO. 252)
EDHVNKKKQHTTSSDQDINE.

The tests disclosed in the Examples in the present disclosure was made using the first full Serratia genome that we recently submitted to NCBI. The chromosomal DNA is accessible under accession code: https://www.ncbi.nlm.nih.gov/nuccore/CP082787.1, and the plasmid used in the tests is available under accession code: https://www.ncbi.nlm.nih.gov/nuccore/CP082788.

In a preferred embodiment, the Contractile injection system of the present invention is a system wherein the Conserved Virulence Cassette is encoded on the pADAP plasmid with GenBank number CP082788.1.

In another preferred embodiment, the Contractile injection system of the present invention comprises Open Reading Frames afp1 to afp16 (SEQ. NO. 57) and is co-expressed with an Open Reading Frame comprising C-terminal truncated afp18 encoding a signal peptide selected from:

(SEQ. NO. 87, 104)
MPYSSESKEKETHSKETERD,
(SEQ. NO. 218)
MPYFNKSKKNEIRPEKSKEE,
(SEQ. NO. 219)
MPYSRESKEKEIHAKETERD,
(SEQ. NO. 220)
MPYFNELNEKETRSKETESG,
(SEQ. NO. 221)
MLYSSESKEKKTHSKETERD,
(SEQ. NO. 222)
MPYFRESKEKDTHAKESKQD,
(SEQ. NO. 223)
MPYSRESKEKDTHAKGSKQD,
(SEQ. NO. 237)
MPYCNASKQKETYLKEIERD,
(SEQ. NO. 224)
MPYSSESKLKDTHLKEAESD,
(SEQ. NO. 205)
MPYTHSKETERDSSESKEKE,
(SEQ. NO. 206)
MPYDRETEKSHTEKEKSESS,
(SEQ. NO. 207)
MDPRYESTSEEKSSEHETKE,
(SEQ. NO. 208)
MRDPYTESSKEESHSKETKE,
(SEQ. NO. 209)
MPYSSESKEKETHSKE,
(SEQ. NO. 210)
MPYSSESKEKETHS,
(SEQ. NO. 211)
MPYSSESKEKET,
(SEQ. NO. 216)
MLPITAKKTNPFQELEQILA,
(SEQ. NO. 217)
MILPTKAKTFNPQEELQILA,
(SEQ. NO. 238)
MNISSYFFLNEENIKFNNQY,
(SEQ. NO. 230)
MLSTEKHNKDTKHPRNREKK,
(SEQ. NO. 231)
MPNSKYSEKVNHSANGAEKC,
(SEQ. NO. 226)
MPRYSNSQRTPTQSTKNTRR,
(SEQ. NO. 253)
MEHEYSEKEKPQGKKPLIKS,
(SEQ. NO. 234)
MEREYSEKEKHKKHPIQLRD,
(SEQ. NO. 235)
MVHEYSINDRQKRHSFSSAN,
(SEQ. NO. 228)
MLKYANPQTVATQRTKNTA,
(SEQ. NO. 239)
LLTHINLIFRVKCKYSICCLF,
(SEQ. NO. 240)
LCAFPIDGYTNERANQGCGE,
(SEQ. NO. 225)
MNISSYFFLNEENIRFNNQC,
(SEQ. NO. 241)
IIFTRDRNPTLSAHIKGGKK,
(SEQ. NO. 230)
MLSTEKHNKDTKHPRNREKK,
(SEQ. NO. 242)
EVNNGGNKSKAQAHTPDLVM,
(SEQ. NO. 243)
ELKHDDSKIKSQVSIPNLVK,
(SEQ. NO. 244)
TIVNPYSIYLKHIPNGFQDA,
(SEQ. NO. 227)
MEHEYSEKEKPQKCPIQLRD,
(SEQ. NO. 245)
LGDDIMPISNLAKESEVRAV,
(SEQ. NO. 232)
MYDSKKKNSEPTTKKKFERS,
(SEQ. NO. 246)
VGNKNTPSRVKIFISALIFM,
(SEQ. NO. 233)
MPNKKYSENTHQGKKPLIKS,
(SEQ. NO. 238)
MNISSYFFLNEENIKFNNQY,
(SEQ. NO. 247)
MPRYANYQJNPKQNIKNSHG,
(SEQ. NO. 248)
MMFDENLECVNEIINEKLD,
(SEQ. NO. 249)
SENKTKHNQQDSTAKDCWHE,
(SEQ. NO. 250)
ESHSDDKHRDQETNQKTANK,
(SEQ. NO. 251)
EDDSKDHFKQSRNQTEKHYN,
(SEQ. NO. 252)
EDHVNKKKQHTTSSDQDINE.

In another preferred embodiment, the Contractile injection system has a minimum of 50% sequence identity to the CVC of the present invention and comprises a packing motif wherein the CC is less than zero.

The contractile injection system described herein can be expressed in any commonly applied expression host selected from a eukaryote cell or a prokaryote cell such as yeast, a bacteria, E. coli, or the native host. In a preferred embodiment, the host is selected from bacteria of E. coli strains, such as One Shot™ BL21 Star™ (DE3), Evo21(DE3), Rosetta; native CIS carrying bacteria like Serratia, Salmonella, Erwinia, Yersinia, or Photorhabdus; or fast-growing strains e.g., Vibrio natriegensis which is the fastest growing free-living bacterium with a doubling time of less than 10 min, which makes it highly attractive as a protein expression host.

The contractive injection system of the present invention as disclosed herein, can be used for different purposes, depending on the effector cargo and the intended target.

With agricultural output predicted to grow exponentially, and the annual loss of 20-40% of crop output to pests and disease, pest control is increasingly becoming cumbersome, as crop pests such as root-knot nematodes, insects and others gain resistance to traditional pesticides at alarming rates. Toxin build-up in the food chain limits the use of synthetic chemical pesticides, the administration of which can cost up to 35 billion dollars annually. Genetically modified crops, including the transgenic varieties of corn, soybean, and cotton, that express Bacillus thuringiensis (Bt) toxin, face reticence due to waning effectivity as pest resistance grows, their unknown long-term effects on human health and the need for organic pesticides like glyphosate to have a substantial protective effect. Highly targeted biocontrol of pests is poised to be a sustainable pest control strategy, ideally with minimal off-target effects, to conserve precious fauna in the eco-system, while protecting agricultural plants. The present contractile injection system provides an alternative to toxins and genetically modified crops for controlling agricultural pests. For instance, the contractile injection system disclosed herein may comprise toxins that are suitable for killing insects and their larvae. Tests are being made wherein some of the constructs disclosed herein comprising the genetically modified Afp1-16 Serratia entomophila syringe (SEQ. NO. 58-73) loaded with for example Casφ-2 (SEQ. NO. 98-113, 151-152) and LL-37 (SEQ. NO. 81-96), respectively, is applied on the Galleria mellonella larvae for showing that the present contractile injection system is effective in killing insects and their larvae. These cargoes are not naturally found in contractile injection systems, LL-37 is a human antimicrobial peptide whereas Casφ-2 (Casphi2) is a procaryotic enzyme providing adaptive immunity against virus infections and plasmid transformations.

In one aspect, the invention provides the use of a contractile injection system according to the invention for controlling agricultural pests, such as bacteria, nematodes, fungi, and insects at any developmental stage.

Targeted delivery of toxins against insects or arthropod larvae also holds immense potential for treatment of e.g., human parasitic infections, which are also major vectors for disease transmission, spreading deadly human viral and bacterial pathogens. Furthermore, as resistance to killing agents develops in species of lice and mites, while drugs to target them in the development pipeline dwindle, the system disclosed herein paves the way towards directed treatment of such infections. Similarly, bacterial infections such as those with opportunistic pathogens including Staphylococcus epidermidis, Staphylococcus aureus and others, especially non-systemic infections of mucosal, lung and skin surfaces are becoming increasingly persistent and resistant to multiple antibiotics. The modified syringe has potential to eradicate specific species of bacteria, while leaving commensal microflora unharmed.

Thus, in one aspect, the contractile injection system according to the invention comprises a medicament or a genetic modification tool as effector cargo for use in delivering a medicament or a genetic modification tool to the target.

The system of the present disclosure can be provided in the form of a composition for treatment of arthropod, insect, or bacterial infections, administered in the form of a topical ointment, gel, spray or edible formulation, protected in liposomes or nanofiber materials. Medicinal formulations can be kept functional through cold storage and have a longer shelf life.

Thus, in yet another aspect, the present disclosure provides a composition comprising a contractile injection system according to the present invention.

### EXAMPLES

### Example 1—Cloning and Production of native Afp Particles (Syringes)

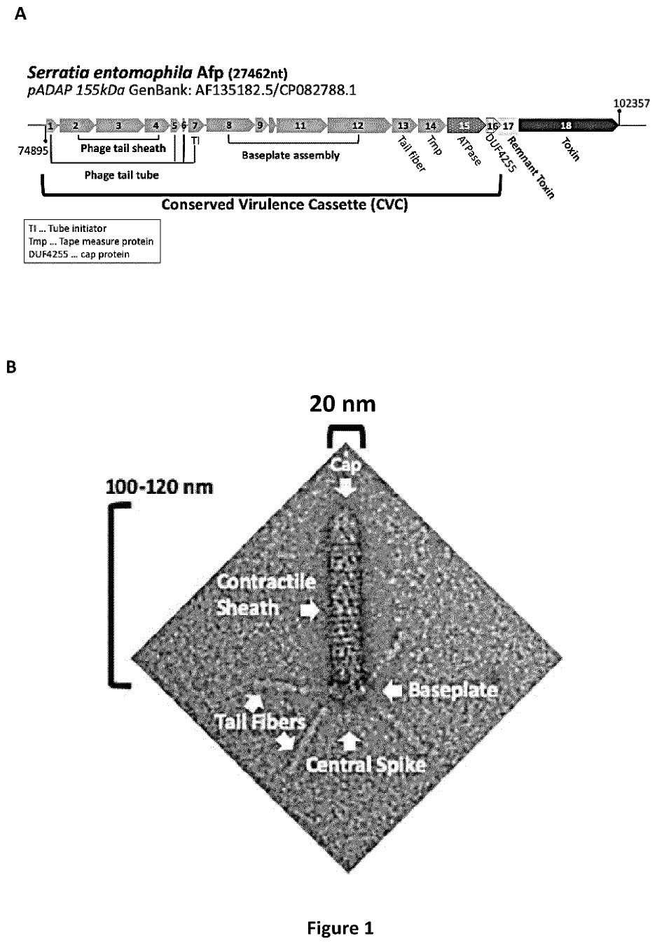

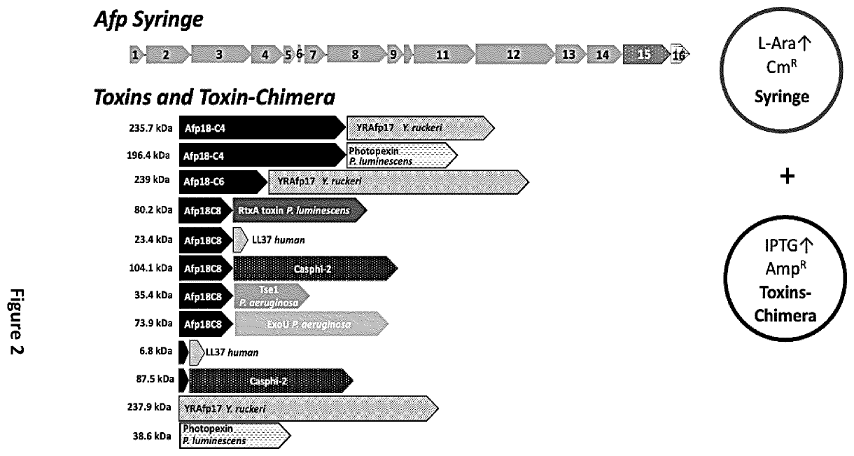

The natural plasmid pADAP (GenBank: AF135182) from Serratia entomophila (Grimont et al., 1988) was prepared with a QIAGEN Plasmid Maxi Kit. The Afp gene cluster, afp1-afp18 (SE1-18) (SEQ. NO. 1), was cloned into a linearized arabinose inducible pBAD33 expression vector (chloramphenicol resistance, CmR) (Guzman et al., 1995) by PCR amplified fragments with overlapping regions into each fragment (FIG. 1). The Afp fragments and the linearized vector, 8.5-15 kb in size, were produced using the Platinum™ SuperFi™ PCR Master Mix (Invitrogen) and fragments gel purified using Monarch® Genomic DNA Purification Kit (NEB: #T3010S). The pBAD33 vector was Dpnl (NEB: #R0176S) digested before gel purification. Fragments were assembled using the In Fusion® HD Cloning Plus CE kit containing DNA in a 1:1:2 ratio including the provided cloning enhancer. The reaction was incubated for 15 min at 37° C., followed by 15 min at 50° C. and 5 μl of the reaction mix was transformed in Stellar competent cells. Positive colonies were screened by colony PCR and restriction digest (BamHI or Xbal and Kpnl) of the plasmid preparations. The SE1-17 cluster (SEQ. NO. 39) was cloned as described for SEAfp1-18 (SEQ. NO. 1). The constructs SE1-18C4 (SEQ. NO. 20) and SE1-16 (SEQ. NO. 57) were cloned the same way as described above, but pBAD33-Afp1-18 (SEQ. NO. 1) and pBAD33-Afp1-17 (SEQ. NO. 39) served as a template, respectively, with 2 equally sized fragments in the In Fusion® assembly mix. Full plasmid sequence was confirmed using Next Generation Sequencing (NGS), showing correct sequence of the whole cluster.

### Example 2—Production of High Quality Afp Syringes

The purpose of this example was to produce syringes with and without cargo, natively expressed (on one plasmid) with high amounts and high quality. These preparations were used as controls for further co-expression and toxin truncation and modification protocols to guarantee particle integrity and quality is kept and toxin levels can be detected and compared to the wild type. The set-up is represented by a one-plasmid expression to yield high syringe amounts, followed by efficient cell lysis, purification through ultracentrifugation and gradient steps. Syringe preparation quality was investigated through three methods, SDS-PAGE, Immunodetection and electron microscopy.

The pBAD33-syringe plasmids obtained in Example 1 were transformed into Electro Competent One Shot™ BL21 Star™ (DE3) with pBAD33 expressing SE1-18 (SEQ. NO. 2-19), SE1-18C4 (SEQ. NO. 21-38), SE1-17 (SEQ. NO. 40-56) and SE1-16 (SEQ. NO. 58-73) and plated on LB-Cm^{R} plates. Colonies were picked and a starter culture of 10 mL LB-Cm^{R} was grown overnight at 37° C. The next morning a growth culture was started in 900 mL media- Cm^{R} and induced at OD600 nm 0.6-0.8 with 0.2% arabinose, grown at 18° C. for 18-22 hours at slow agitation.

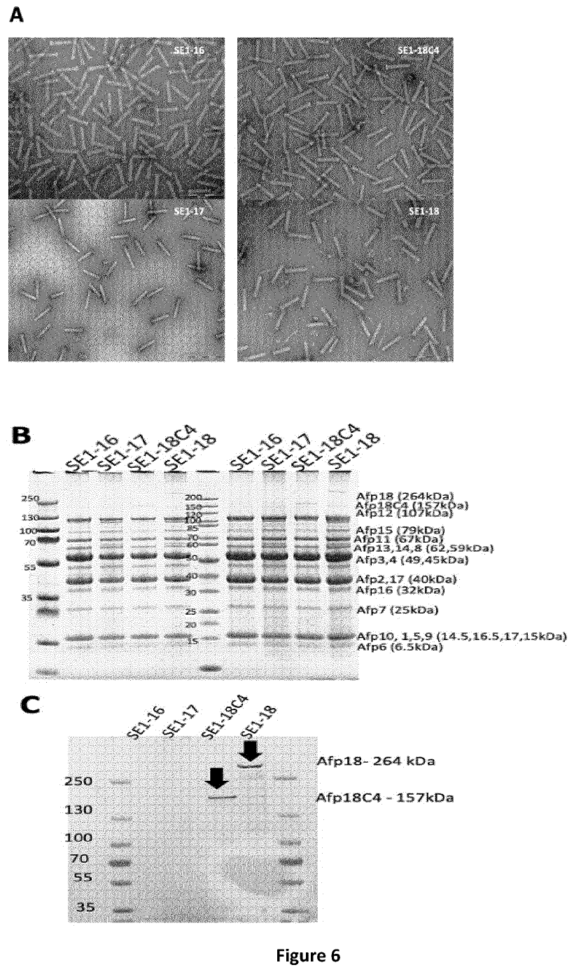

After induction, cells were harvested and resuspended in 25 mL of lysis buffer (25 mM Tris pH 7.4, 140 mM NaCl, 3 mM KCl, 200 μg/mL lysozyme, 50 μg/mL DNase I, 0.5% Triton X-100, 5 mM MgCl₂ and one tablet cOmplete™ Protease Inhibitor Cocktail from Roche) and incubated for 45 min at 37° C. The lysate was cleared for 45 min, 4° C. and 18,000×g centrifugation. After clearing the lysate the syringes were pelleted in two ultracentrifugation (UC) rounds, each 45 min, 4° C., 150,000×g and resuspended first in 5 mL then in 0.5 mL 1×PBS buffer. After the second round of UC the syringes were loaded on an OptiPrep™ gradient ranging from 40%, 35%, 30%, 25%, 20% and 10% prepared in 1×PBS and run for 20-24 h, at 4° C. at 150,000×g. Fractions were harvested in 0.5 mL steps and syringe location confirmed over SDS PAGE. Syringe Samples were pooled and dialyzed for 6 days at 6° C. in 1×PBS and after that a last round of UC was performed and the syringes resuspended in 0.5 mL of 1×PBS. Quality of syringes was investigated over negative staining electron microscopy (EM) (FIG. 6 (A)), SDS PAGE (FIG. 6 (B)) and Immunodetection Western blotting (FIG. 6 (C)). Particle quality and toxin levels were used as references for further experiments.

### SDS-PAGE Analysis

The syringes were diluted in 1×PBS to equal concentrations for comparison on SDS-PAGE and Coomassie staining and for immunodetection blots. The samples were supplemented with reducing Laemmli SDS sample buffer (250 mM Tris-HCl, 8% SDS, 40% Glycerol, 8% β-merceptoethanol, 0.02% Bromophenol blue, pH 6.8), boiled for 5 min at 98° C., centrifuged at 14,000×g for 2 min and loaded on Invitrogen™ NuPAGE™ 4 to 12%, Bis-Tris gels and proteins resolved at 200 V for 40 min. The gels were stained with Instant Blue™ Coomassie Stain for 30 minutes and washed with water for several hours before evaluation.

### Immunodetection Blot Analysis

The syringes were diluted in 1×PBS to appropriate concentrations for visualization for SDS-PAGE and following Immunoblotting and detection by toxin and syringe specific antibodies. The samples were prepared as described above (SDS-PAGE analysis) using Invitrogen™ NuPAGE™ 4 to 12%, Bis-Tris gels (for syringes) or Invitrogen™ NuPAGE™ 3 to 8%, Tris-Acetate (for high molecular weight toxin analysis). The NuPAGE™ 4 to 12%, Bis-Tris gels were run as described above and the NuPAGE™ 3 to 8%, Tris-Acetate was run at 150V for 70 minutes. Afterwards gels were removed from the plastic shields, washed in water. NuPAGE™ 3 to 8%, Tris-Acetate gels were soaked for 10 min in 20% Ethanol to allow increased toxin protein blotting. Proteins from respective gels were transferred on iBlot™ Transfer Stack, PVDF membranes for 7 minutes for Bis-Tris gels and for 10 min for Tris-Acetate gels using Invitrogen™ iBlot® Dry Blotting System. The membrane was washed in water and exposed to syringe and toxin specific antibodies using iBind™ Western Devices. Antibodies and Western reagents were prepared using the Invitrogen™ iBind™ Solution Kit with antibody dilutions ranging from 1:100 and 1:1000. Membranes were exposed to TMB-D Blotting Solution (Kementec) followed by scanning and analysis.

### Negative Staining Electron Microscopy

Syringe preparations were diluted in 1×PBS to reach around 0.2 mg/mL concentration using Bio-Rad Protein Assay Dye Reagent and spectrophotometric measurements at 595 nm (Bradford). A 4 μl syringe dilution sample is pipetted on freshly glow discharged (Balzers Union dual chamber CTA 010 glow discharger) onto copper grids (200 mesh) coated with continuous carbon, washed, and stained with 2% uranyl acetate. The grids were dried at room temperature and analyzed on a Morgagni 268 transmission electron microscope at 100 kV and syringes investigated for sample purity and assembly state.

FIG. 6 shows natively expressed (Single Plasmid Expression) and purified syringe particles for validation of syringe quality and toxin presence in native and truncated form for comparison to following co-expression protocols. The native syringe SE1-18 (SEQ. NO. 2-19), the syringe missing all toxins SE1-16 (SEQ. NO. 58-73), the syringe including one remnant toxin SEAfp17 (SEQ. NO. 56), SE1-17 (SEQ. NO. 40-56), and a syringe version carrying half of SEAfp18 (SEQ. NO. 38), SE1-18C4 (SEQ. NO. 21-38), proofing that syringe quality and toxin levels are comparable to native wild type production. (A) Negative staining electron micrographs of intact syringes at purest state. All particles show very similar dimensions and have all necessary features. (B) Syringes resolved on SDS PAGE investigated for protein species presence at low (1:10 diluted, left) and high (right) particle concentration. (C) Immunodetection blot confirming the presence of the SEAfp18 (264 kDa, SEQ. NO. 19) toxin and the truncated version SEAfp18C4 (157 kDa, SEQ. NO. 38) in comparable amount and intact status.

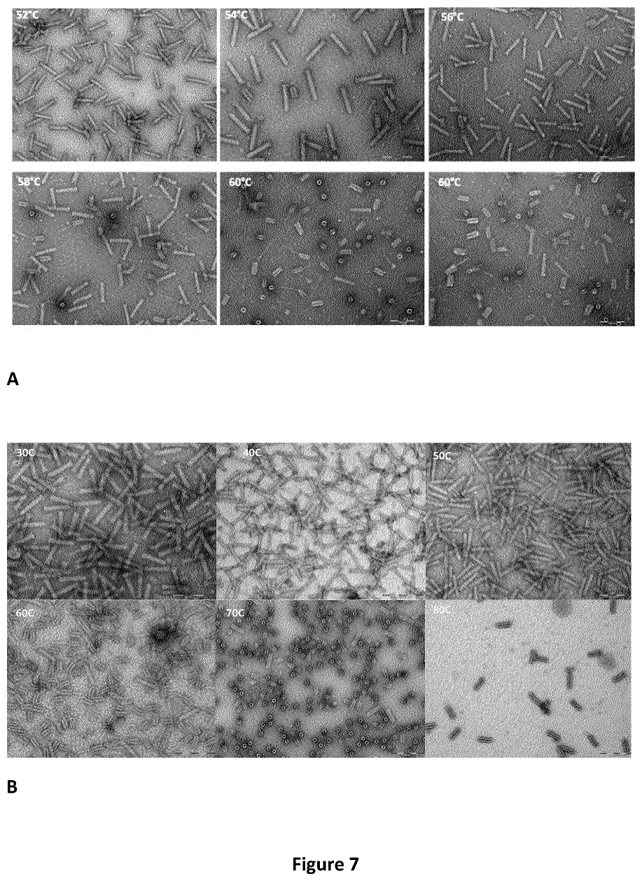

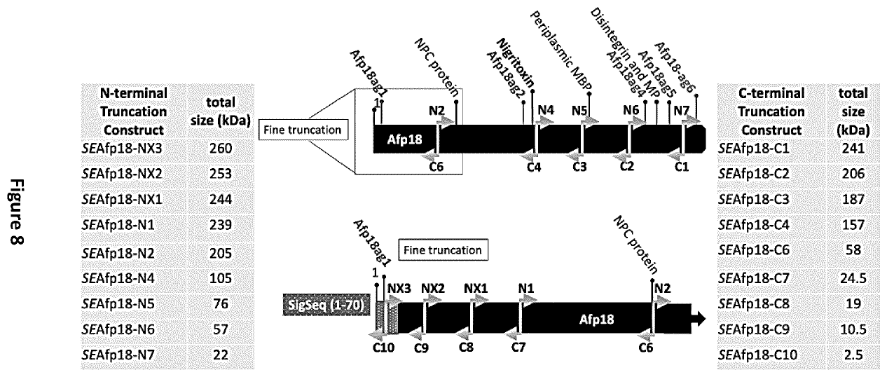

Ejection capability and stability of the obtained syringes was tested using heat treatment gradient (FIG. 7). Stability and ejection were monitored by exposing particles to heat at temperatures ranging from 30-80° C. Proper Syringe ejection was seen at a temperature as high as 58-60° C. The syringes were exposed to each specific temperature for 10 minutes in 1×PBS buffer and a sample was analyzed on negative staining EM. The syringes showed remarkable temperature stability in their native conformation including the Afp18 toxin (SE Afp18, SEQ. NO. 19). This stability behavior was used as a reference for our modified syringes with alternating toxins. FIG. 7 (A) shows 20 μl samples at 1:40 dilutions in 1×PBS, 10 min. at respective temperature, 4 μl samples were used for the staining. Fine temperature gradient was found between 52-60° C. Most syringes stayed intact until 56° C. At 58-60° C. the ejection state can be observed, the syringe is in a compact sheath state and the long tube is penetrating through the baseplate.

FIG. 7 (B) shows 20 μl samples at 1:15 dilutions in 1×PBS, 10 min. at respective temperature, 4 μl samples were used for the staining. A larger temperature gradient was used for the samples shown in FIG. 7 (B) and visualizes syringe behavior beyond ejection temperature. At 70° C. the syringes were degraded, and the sheath-tube complex was visible from the top (ring structure). At 80° C. most of macromolecular syringe assembly structures were degraded.

The present tests showed that extremely large CIS clusters can be cloned, with perfect sequence match, on expression vectors for high production levels in bacteria. The syringes in variants with toxin and without toxin were produced in high quality and toxin presence in full and truncated form was detected with similar quality and amount. Truncation of the Afp18 toxin from 264 kDa to 157 kDa (SEQ. NO. 38) did not affect native syringe and toxin level and quality production (FIG. 6). Functionality of syringes was investigated over temperature induced ejection (T_{ejection}=58° C.) and negative staining electron microscopy (FIG. 7) representing high thermal stability.

### Example 3—Cloning and Co-Production of Syringes with Toxins, Afp18-Toxin and Afp18-Effector Chimera

The purpose of this Example was to develop a high throughput syringe purification protocol to analyze as many conditions as possible and being suitable for robotic set. The developed quick-purification protocol allows assessment if a cargo is successfully attached to the syringe or whether it is present as a soluble aggregate. A mock expression of cargo, without syringe samples, visualizes whether cargo is only present if the syringe is produced confirming co-purification of syringe and cargo.

We investigated whether the Afp18 toxin is truncate-able and whether truncation at the C-terminus or the N-terminus works best. We also tested whether parts of Afp18 toxin be replaced by other toxins for multi-toxin delivery and whether Afp18 can be used to deliver other secretion system effectors with therapeutic potential. Finally, we identified the required packing motif to attach cargo that is not related to natural syringes

The set-up is represented by a flexible co-expression approach (FIG. 2), where the syringe was on one plasmid and cargo could be fast exchanged on a smaller plasmid. Syringe and cargo were co-expressed and purified and the established protocol, with no syringe control of cargo, revealed whether cargo was attached to the syringe. Syringe preparation quality was investigated through three methods, SDS-PAGE, immunodetection and electron microscopy.

### Cloning of Full Toxin and Toxin Truncation Constructs for Co-Expression and Purification

The afp18 toxin gene was amplified from pADAP plasmid (GenBank: AF135182.5, GenBank: CP082788.1) DNA preparations by PCR using the In-Fusion® assembly mix, into an ampicillin resistant pET11a vector, creating Afp18 (untagged, SEQ. NO. 74) and Afp18-3CTS (SEQ. NO. 178), with a C-terminal Twin Strep Tag (3CTS) (FIG. 2, and Table 2). Positive clones were confirmed with colony PCR, restriction digest and perfect sequence validated by NGS. The afp18 homolog, afp17 (YR17, SEQ. NO. 76) encoded on the Yersinia ruckeri ATCC 29473 genome, was cloned and validated in the same way than afp18 but using genomic Y. ruckeri DNA as a template prepared with a Sigma gDNA GenElute® Bacterial Genomic DNA Kit (Sigma-Aldrich). The Photopexin toxin PluDJC_08520 (SEQ. NO. 78), part of a Photorhabdus luminescens DJC CIS cluster, was cloned and validated as described above (Photorhabdus luminescens DJC was kindly provided by Prof. Ralf Heermann, University of Mainz).

To investigate if the N-terminal or C-terminal part of Afp18 is responsible for cargo packing and which part locates the packing motif we designed a series of N- and C-terminal truncation variants (SEQ. NO. 114-148) and truncation border chosen based on secondary and tertiary structure prediction programs Quick2D and HHPRED respectively, provided by MPI Bioinformatics Toolkit (https://toolkit.tuebingen.mpg.de). The Afp18 truncation constructs were purchased from GenScript Gene Cloning Services providing Afp18 plasmid (SEQ. NO. 74) as a template. The design and strategic toxin segmentation generating N- and C-terminal Afp18 toxin truncation variants for syringe-toxin co-expression are shown in FIG. 8 and were made in accordance with the experimental design shown in FIG. 2.

Bioinformatic analysis and prediction (HHPRED and Quick2D from the toolkit https://toolkit.tuebingen.mpg.de/) revealed homologous protein domains along Afp18 toxin (black pins shown in FIG. 8). Toxin truncations and respective rabbit polyclonal toxin antibodies (pAB, ag1-6, grey pins shown FIG. 8, SEQ. NO. 200) were designed for toxin detection along the production process. A fine truncation around the predicted ‘unknown’ signal sequence (SigSeq 1-70 amino acids) using SignalP—6.0 server was performed to reveal if the Afp18 N-terminus is responsible for packing and which amino acids. Sizes of constructs are moreover presented in FIG. 8 for the N-terminal and C-terminal truncations.

An unknown signal sequence was predicted for the amino acids 1-70 at the N-terminus using the latest version of the Signal-P 6.0 server (DTU Health Tech, https://services.healthtech.dtu.dk/service.php?SignalP-6.0).

TABLE 2
Toxin and Toxin-Chimera Construct Overview

| | | | | Co-Purified |
|---|---|---|---|---|
| | | | thereof | with Syringe |
| | | Afp18 | Toxin/Effect | Yes/Not |
| | total | fragment | or | Detected |
| Construct | size (kDa) | size (kDa) | size (kDa) | (ND) |
| S. entomophila (SE) Afp18 (SEQ. NO. 75) | 264 | 264 | — | Yes |
| Y. ruckeri (YR) Afp17 (SEQ. NO. 77) | 237.9 | — | 237.9 | Yes |
| P. luminescens Photopexin (SEQ. NO. 79) | 38.6 | — | 38.6 | Yes |
| Afp18C4-YRAfp17 (1437-2123, C4-YR) | 235.7 | 157.8 | 77.4 | Yes |
| (SEQ. NO. 198, 199) | | | | |
| Afp18C6-YRAfp17 (502-2123, C6-YR) | 239 | 58 | 181 | Yes |
| (SEQ. NO. 195, 196) | | | | |
| Afp18C4- P. luminescens Photopexin | 196.4 | 157.8 | 38.6 | Yes |
| PluDJC_08520 (C4-PL) (SEQ. NO. 192, | | | | |
| 193) | | | | |
| Afp18C4-sfGFP (SEQ. NO. 187-190) | 205.6 | 157.8 | 47.8 | Yes |
| Afp18C6-sfGFP (SEQ. NO. 182-185) | 105.8 | 58 | 47.8 | Yes |
| Afp18C8- P. luminescens RtxA toxin | 80.2 | 19 | 61.2 | Yes |
| PluDJC_12685 (C8-RtxA) (SEQ. NO. 170, | | | | |
| 175) | | | | |
| Afp18C10-- P. asymbiotica YopT-RHS toxin | 145 | 3.5 | 36.9/105 | YopT Yes |
| PAU_RS10125-20 (C10-YopT-RHS) (SEQ. | | | | RHS ND |
| NO. 166-168) | | | | |
| Afp18C6-Cas9 (SEQ. NO. 163, 164) | 216 | 58 | 158.4 | ND |
| Afp18C8-LL37 human antimicrobial peptide | 23.4 | 19 | 4.4 | Yes |
| (SEQ. NO. 160, 161) | | | | |
| Afp18C8-P. aeruginosa Type VI SS Effector | 35.4 | 19 | 16.4 | Yes |
| Tse1 (PATse1) (SEQ. NO. 157, 158) | | | | |
| Afp18C8- P. aeruginosa Type III SS Effector | 92.9 | 19 | 73.9 | Yes |
| ExoU (PAExoU) (SEQ. NO. 154, 155) | | | | |
| Afp18C8-Biggiephage Casphi2 (C8- | 104.1 | 19 | 85.1 | Yes |
| Casphi2) (SEQ. NO. 151, 152) | | | | |
| Afp18NT20-Biggiephage Casphi2 (C20- | 87.5 | 2.4 | 85.1 | Yes |
| Casphi2) (SEQ. NO. 104, 105) | | | | |
| Afp18NT20- LL37 human antimicrobial | 6.8 | 2.4 | 4.4 | Yes |
| peptide (SEQ. NO. 87, 88) | | | | |
| Afp17-Afp18 (17-18) (SEQ. NO. 173, 174) | — | 40/264 | — | Afp17 ND |
| | | | | Afp18 Yes |
| Afp18-sfGFP (SEQ. NO. 176, 177) | 290.8 | 264 | 26.8 | Yes |
| Afp18-C-terminal Twin Strep Tag (C3CTS) | 268.3 | 264 | 4.3 | Yes |
| (SEQ. NO. 179, 180) | | | | |

Table 2 shows Toxin, Toxin-Chimera and Toxin-Effector Constructs and results of co-purification with the syringe. Two examples, Y. ruckeri YR17 (SEQ. NO. 77) and P. luminescens Photopexin (SEQ. NO. 79), where co-purified without Afp18 as scaffold, RHS toxin (SEQ. NO. 168) and Afp17 (SEQ. NO. 173) remnant toxin are two examples of not detectable (ND) toxin co-purification with the syringe.

### Cloning of Afp18-Toxin Constructs for Co-Expression and Purification:

To investigate whether toxins of Afp related CIS can be fused and co-purified with Afp18 we designed a set of Afp18-toxin-chimeras (FIG. 2, Table 2). For cloning of homologous effectors, genomic DNA of Photorhabdus luminescens DJC, Photorhabdus asymbiotica ATCC43949 and Y. ruckeri ATCC 29473 gDNA was purified based on a phenol-chloroform based protocol (Wang et al., 2016). The toxin genes PluDJC_08520 (Photopexin), PluDJC_12685 (RtxA toxin) from P. luminescens DJC, the afp18 homologue DJ39_RS03245 (YRAfp17) from Y. ruckeri, were fused to the 3′end of C-terminally truncated Afp18-C4(11437), Afp18-C6(T171), Afp18-C10(S30). These toxin chimeras (SEQ. NO. 169, 165, 191) were produced by linearizing truncated Afp18 C-truncated plasmids and PCR amplifying selected toxins with 20nt overhangs into the Afp18 vectors. The Afp18-toxin chimeras were fused with the In Fusion® assembly mix, clones screened and confirmed as described for cloning of Afp18 (SEQ. NO. 74).

### Cloning of Afp18-Effector Constructs for Co-Expression and Purification

The limit of Afp18 truncations to serve as a scaffold for co-purification and delivery of effector molecules was screened by fusing Afp18 C-truncation constructs to other secretion system effectors and to non-CIS related cargo, a short antimicrobial peptide (AMPs) and to a biggie phage Caso-2 (FIG. 2, Table 2). We ordered synthesis and subcloning of Type VI secretion system effectors of Pseudomonas aeruginosa PAO1, tse1 (gene: PA1844, Uniprot: Q912Q1) (SEQ. NO. 156), Type III secretion system effectors of Pseudomonas aeruginosa UCBPP-PA14, exoU (gene: exoU, Uniprot: 034208) (SEQ. NO. 153), codon-optimized (for bacterial expression) CasO-2 of Biggie phage (Pausch et al., 2020) (SEQ. NO. 159), a short non-CIS related AMPs, human LL-37 (Uniprot: P49913, ‘LLGDFFRKSKEKIGKEFKRIVQRIKDFLRNLVPRTES’) Afp18-C8 from Genscript (FIG. 2). The two non-CIS cargos LL-37 and Casφ-2 (Casphi2) were attached to the designed Afp18 N-terminal constructs including the first 50, 30, 20, 11, 5 and 2 amino acids (SEQ. NO. 80-112), subcloning was ordered from Genscript.

### Co-Expression and Purification of Afp Syringes and Cargo

Chemically competent One Shot™ BL21 Star™ (DE3) with pBAD33 expressing SE1-18 (SEQ. NO. 1), SE1-17 (SEQ. NO. 39) and SE1-16 (SEQ. NO. 57) were prepared using a rubidium-chloride based standard protocol. Afp18, Afp18-toxin chimeras and Afp18-effector chimeras (SEQ. NO. 74-78, 114-197) were transformed into chemically competent SE1-17 and SE1-16 respectively and colonies selected for chloramphenicol and ampicillin resistance (CmR, AmpR) as shown in FIG. 2. As a control experiment the Afp18 toxin constructs were expressed without Afp particles, termed mock expression—‘no syringe’ samples—to monitor toxin co-purification or insoluble toxin purification. For high throughput co-expression studies 200 mL of each plasmid combination was cultured and induced at OD600 nm 0.6-0.8 with 0.25 mM IPTG 30 min prior to 0.2% arabinose, grown at 18° C. for 18-22 hours at slow agitation. The co-expression protocol was optimized for balanced IPTG/arabinose concentrations leading to a detectable toxin:particle ratio. After induction, cells are harvested and resuspended in 3 mL of lysis buffer (25 mM Tris pH 7.4, 140 mM NaCl, 3 mM KCl, 200 μg/mL lysozyme, 50 μg/mL DNase I, 0.5% Triton X-100, 5 mM MgCl₂ and one tablet cOmplete™ Protease Inhibitor Cocktail from Roche) and incubated for 45 min at 37° C. The lysate is cleared for 45 min, 4° C. and 18,000×g centrifugation. After clearing the lysate are precipitated with 8% polyethylene glycol (PEG) 6,000 and 0.5M NaCl and slowly agitated overnight in the cold-room (6-10° C.). The next day particles were collected with a centrifugation at 4,000×g for 20 min at 4° C. and the pellet resuspended in 1 mL ice cold 1×PBS buffer and agitated for 4 h in the cold room. Afterwards remaining precipitation was pelleted for 45 min at 14,000×g and supernatant saved for analysis on SDS-PAGE. Afterwards the supernatant was ultracentrifuged 150,000×g for 45 min at 4° C. to pellet the particles.

The samples were then investigated on Coomassie, immunodetection blots and electron microscopy for particle and cargo validation as described in Example 2 (SDS-PAGE, Immunodetection Blots, and electron microscopy).

### Results and Conclusion

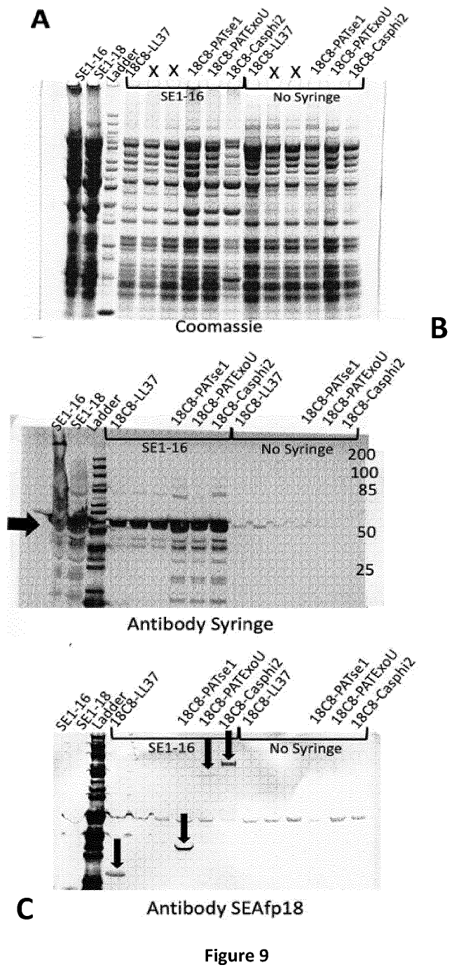

This protocol was established to be economic, fast with minimal material to visualize whether a cargo is attached to the syringe or not. As a control the effector cargo (toxins, toxin-chimera, toxin-effectors) was produced without the syringe (‘no syringe’ samples, FIGS. 9 and 11) to show if there are soluble aggregates to verify that no background precipitation, only cargo associated to syringes, was purified.

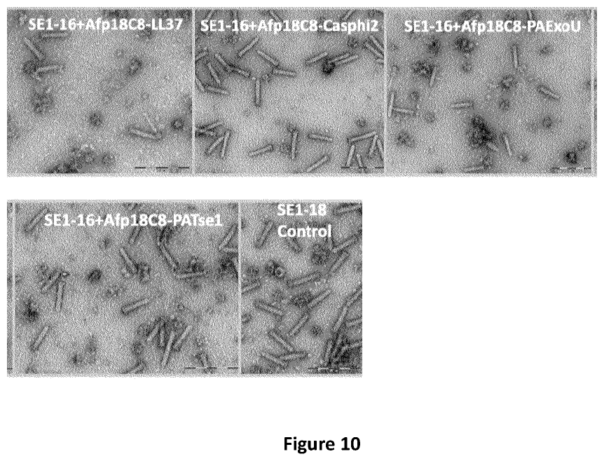

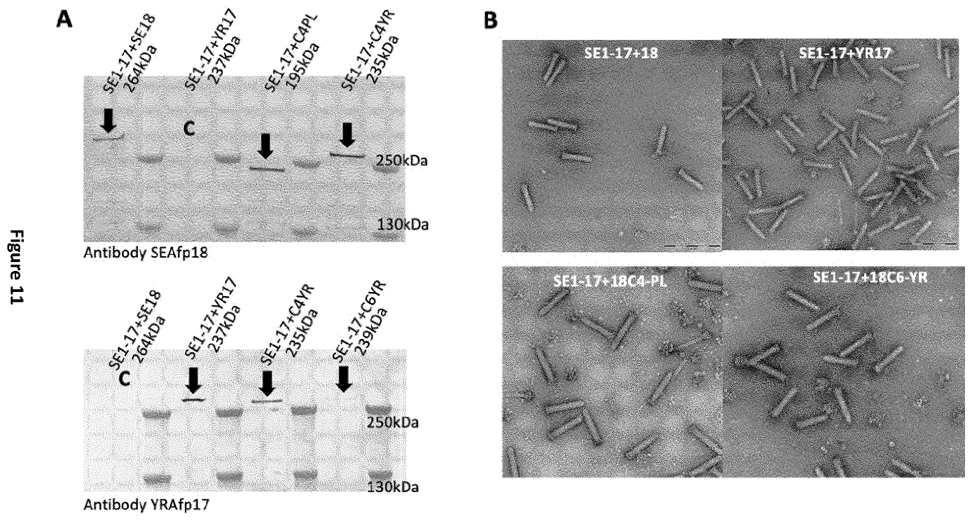

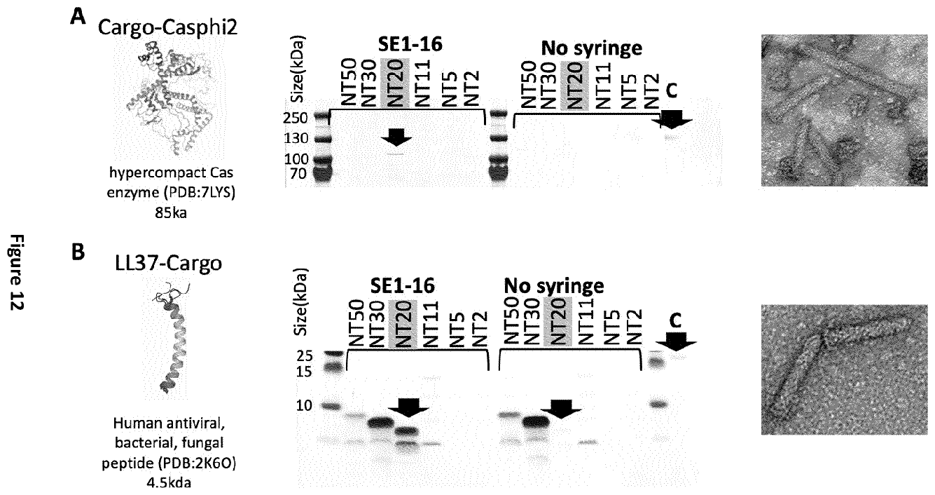

FIGS. 9 and 10 shows the syringe and toxin-effector co-production and purification validated by SDS PAGE (FIG. 9 (A)), WESTERN Blot (FIG. 9 (B) and (C)) and negative staining electron microscopy (FIG. 10).

The Afp18C8 (19 kDa) (SEQ. NO. 147) truncation was used to show that non syringe related effector cargo attachment of a human antimicrobial, antifungal, antiviral peptide, LL37 and Casphi2 a hypercompact gene editing enzyme from Biggiephage, can be attached to the syringe with using the Afp18 N-terminus as a scaffold (SEQ. NO.151, 152 & 160, 161). Type III and Type VI Secretion System (SS) effectors ExoU and Tse1 show also successful packing (SEQ. NO. 154-158). In FIG. 9 (A), (B) and (C) Syringes purified with toxin-effector, verified over Coomassie and immunodetection using syringe and toxin specific antibodies. The presence of syringes and effectors attached to the syringe are highlighted with black arrows. As a control the toxin-effector constructs are purified in parallel, ‘no syringe’ samples, to proof that toxin-effectors are only present when the syringe purified. In FIG. 10 the syringe quality is shown in negative staining electron microscopy. Integrity, stability, and features of the syringe are present as in the wild type controls SE1-18. As seen from FIGS. 9 and 10, the N-terminus of Afp18 can be used to attach other secretion system effectors (Tse1, ExoU) and Afp18 can thus be used to deliver other secretion system effectors with therapeutic potential.

The applied protocol allows production of syringes to a level that can visualize reliable cargo attachment to good quality syringes (FIGS. 9-12). The samples are in good quality to be analyzed for high resolution cryogenic electron microscopy.

FIG. 11 shows syringe and toxin-chimera co-production and purification validated by immunodetection (A) and negative staining electron microscopy (B). The native Afp syringe production was simulated in the above co-expression approach expressing the syringe SE 1-17 (SEQ. NO. 40-56) plus native untagged Afp18 toxin (SEQ. NO. 75) and Afp18-Toxin Chimeras (FIG. 2, Table 2) (SEQ. NO. 195-199). FIG. 11 (A) it is seen by immunodetection that toxin and toxin chimeras co-purified with the syringe. Native Y. ruckeri Afp17 (YR17) (SEQ. NO. 77) was natively co-purified with the S. entomophila syringe, and the toxin can be detected with a Y. ruckeri Afp17 specific antibody (YR17ag1) (SEQ. NO. 204). The toxin-chimeras, consisting of Afp18-C4(11437) and Afp18-C6(K526) fused to either parts of YR17 (SEQ. NO. 195-199) or P. luminescens Photopexin (PluDJC_08520) (SEQ. NO. 192, 193), indicated with C4-YR, C6-YR and C4-PL respectively, were co-purified with the syringe. Lanes marked with (C), highlight the negative antibody control, where the antibody is out of range, therefore no toxin detection should be visible. FIG. 11 (B) shows negative staining electron microscope images as a syringe quality and control to visualize fully assembled syringes with all syringes features present (see also FIG. 1B). The N-terminus (in various lengths) of Afp18 can thus be used to attach other toxins to the syringe (YRAfp17, Photopexin) and can therefore be concluded that parts of Afp18 toxin be replaced by other toxins for multi-toxin delivery.

FIG. 12 shows identification of minimal packing motif using non-syringe related cargo Casphi2 and LL37. Non-syringe related cargo Casphi2 (A) and LL37 (B) were attached to the first 50, 30, 20, 11, 5, and 2 amino acids of the crucial Afp18 N-terminus (AfpNT) (SEQ. NO. 81-113) to find a minimal packing motif. In panel A and B from left to right, structure representation of the cargo attached, mid immunodetection of Afp18NT-effector purification including control purification of effector only (no syringe) and right negative staining electron microscope images of intact syringes. Syringes are complete and intact and Afp18NT20 (SEQ. NO. 87, 104) is highlighted (grey shaded) as minimal packing motif for Casphi2 and LL37 to be packed to the syringe without background purification (see no syringe band of immunodetection). A control (C) was loaded on each immune detection blot.

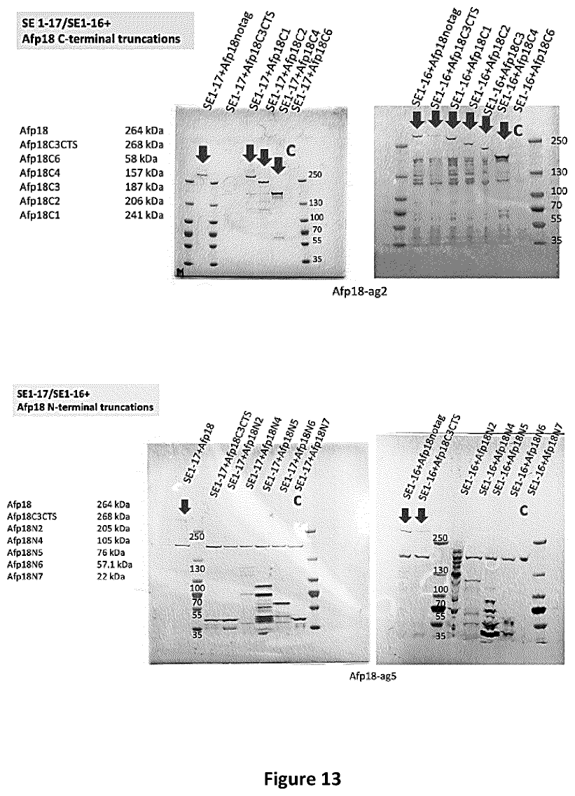

Our results show that the N-terminus (C-terminal truncations (SEQ. NO. 81-123, 143-149)) represents a stable scaffold for manipulation and attachment to attach cargo to the syringe (FIGS. 8 and 13). The N-terminal truncations (SEQ. NO. 125-141) show high toxin degradation indication important sequence features located at the N-terminus (FIGS. 13-16).

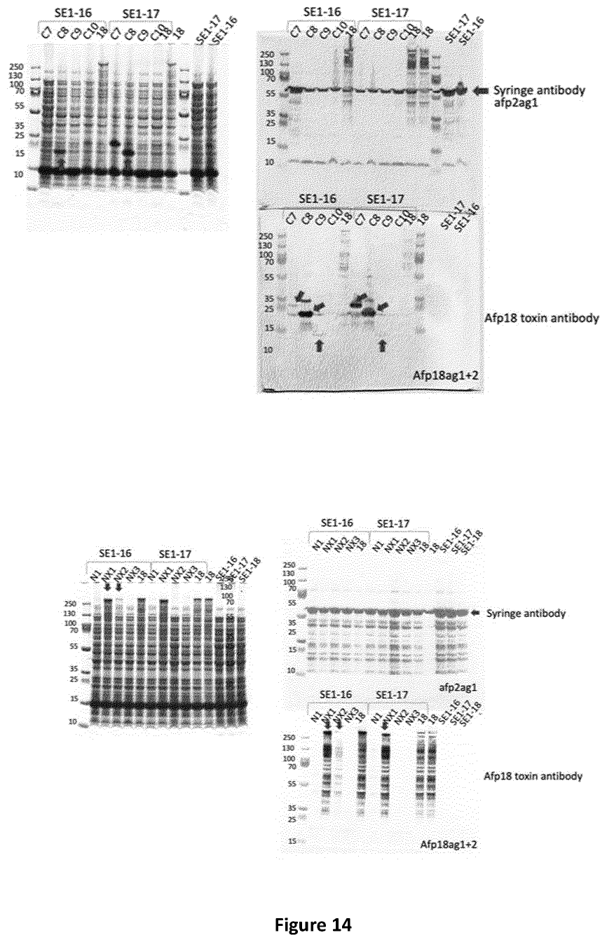

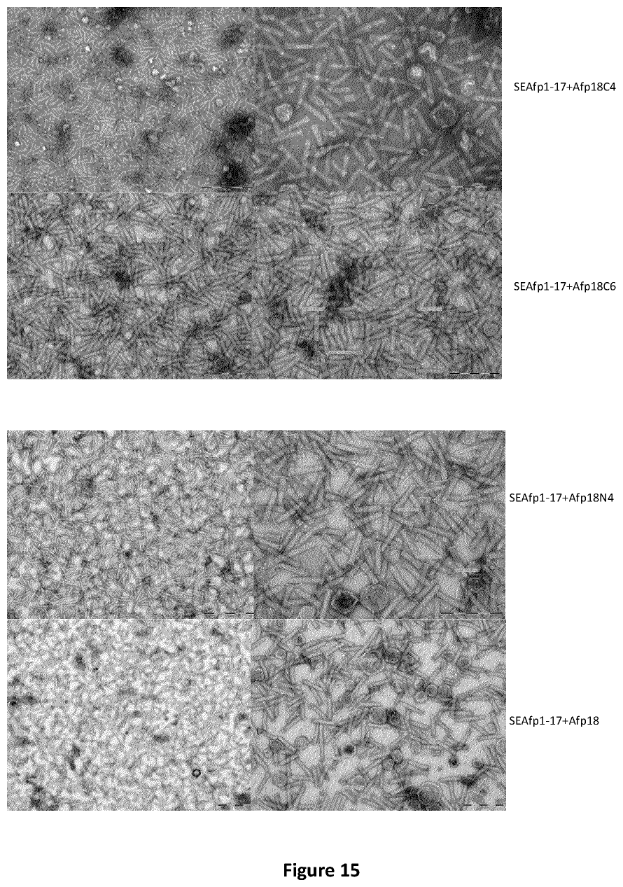

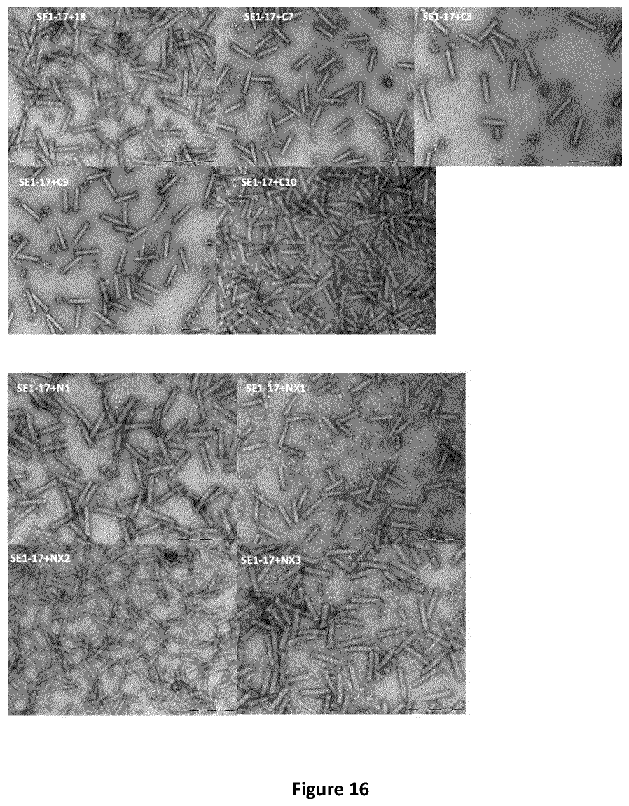

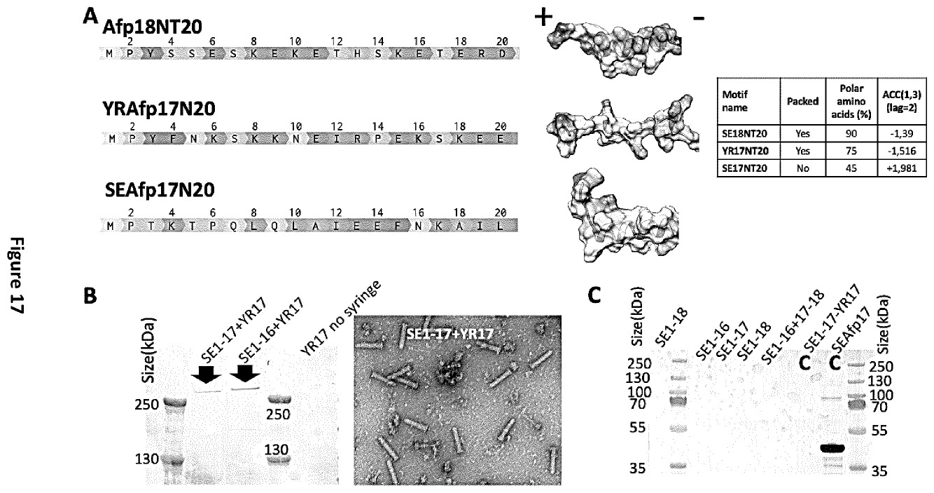

FIG. 13 shows that the Afp18 toxin C-terminus truncations (SEQ. NO. 81-123, 143-149) allowed stable syringe-toxin purification. Immunodetection blots visualized the stable and degraded presence of Afp18 truncation variants with co-expressed and purified syringes, SE1-17 (SEQ. NO. 40-56) and SE1-16 (SEQ. NO. 58-73), with C-terminal truncated Afp18 toxin (top) and N-terminal truncated Afp18 toxin (bottom), using Afp18 specific pABs (SEQ. NO. 200). Toxin stability is increased when truncated from C-terminus (top, arrows). N-terminal truncation of Afp18 showed high toxin degradation (bottom). Purification of a larger Afp18 toxin, Afp18C3CTS (SEQ. NO. 179, 180) including a non-structured C-terminal Twin Strep tag, could be detected when co-expressed with SE 1-16 but not with SE1-17, indicating a toxin size increase is possible if Afp17 is missing. Lanes marked with (C), highlight the negative antibody control, where the antibody is out of range, presenting background signal. Syringes or all truncations are intact (FIGS. 15-16) and the C-terminal truncation Afp18C9 (SEQ. NO. 145) was (10.5 kDa) the smallest truncation detectable (FIG. 14) N-terminal truncation leads to heavy toxin degradation profiles.

FIG. 14 shows co-expression of Afp Syringes SE 1-16 (SEQ. NO. 58-73) and SE 1-17 (SEQ. NO. 40-56) with N- and C-terminal fine truncated Afp18 toxins. We investigated expression levels for the Afp syringe and the N- and C-terminally truncated Afp18 toxin over SDS PAGE (left) and WESTERN Blot (right) with syringe (afp2ag1) (SEQ. NO. 202, 203) and toxin specific (Afp18ag1-6) (SEQ. NO. 200) pABs. C-terminal truncations (top) show similar syringe expression for all constructs and toxin over expression upregulated until truncation C9 (A95) (SEQ. NO. 145). However, Afp18 truncation C8 (T171) (SEQ. NO. 147) shows highest toxin levels. For N-terminal Afp18 truncation constructs expression (bottom) was detected at respective positions NX1 (M172) (SEQ. NO. 137) and NX2 (A96) (SEQ. NO. 139), however for truncation Afp18 NX2 only very low toxin levels were detected and only when co-expressed with SE1-16. Additionally, substantial toxin degradation is visible for N-terminal truncated variants. The native syringe, SE1-17, SE1-16 and native Afp18 toxin were expressed as controls. The lowest detectable Afp18 C-terminal truncation was shown to be Afp18C9 (SEQ. NO. 145) with 19 kDa, as Afp18 toxin size detection limit.

FIGS. 15 and 16 shows co-expressed and purified syringes including Afp18 toxin N- and C-terminal truncation variants investigated with electron microscopy (EM). Syringes with modified toxins were investigated for size and shape. Co-expression SE1-17+Afp18 (SEQ. NO. 40-56, 75) is simulating native syringe and serves as a control. No obvious size or shape differences can be observed for both N- and C-terminal Afp18 toxin co-expressed with the syringe. The syringe integrity and quality were thus not affected by either N-terminal or C-terminal truncation of the Afp18 toxin.

In FIG. 17, the Identifications of packing motifs are shown together with an example of a signal peptide that was found not to comprise a packing motif. FIG. 17 comprises examples of two CIS syringe cargos and their N-terminal packing motifs, Afp18NT20 (SEQ. NO. 87, 104), YRAfp17NT20 (SEQ. NO. 218, 254) that showed successful packing and SEAfp17N20 (SEQ. NO. 173, 269) which did not pack. FIG. 17 (A) shows the three signal peptides description and comparison on sequence, structural level and comparison of packing motif parameters (table). Afp18NT20, YRAfp17NT20, show similar structure with high amount of positively and negatively charged residues (grey structure parts), high amount of polar amino acids, 90% and 75% and the CC (1,3; lag=2) values are negative. An example of a not qualified packing motif is SEAfp17N20 (SEQ. NO.173), which has bulky structure, low amount of charged and polar amino acids and the CC (1,3; lag=2) is highly positive with +1.981. FIG. 17 (B) is a confirmation of cargo presence of immunodetection with antibodies (black arrows) and intact syringes on negative staining electron micrograph. FIG. 17 (C) shows the signal peptide of SEAfp17N20 (SEQ. NO. 213) did not lead to packing of SEAfp17 (SEQ. NO. 173) as visualized by immunodetection (SEQ. NO. 201). Respective positive (SEAfp17) and negative (SE1-17+YR17, SE1-16) controls were included. SEAfp17 could not be detected in any co-production set up (SE1-18, SE1-17, SE1-16+17-18) where it should be present if the signal peptide had included a packing motif.

### Example 4—Database Analysis Applied for Defining the Characteristics of the Packing Motif

Based on the results from Examples 1, 2 and 3 we applied database analysis in order to identify conserved N-terminal packing motifs in homologous CIS syringes.

Using the SEAfp18NT20 (SEQ. NO. 87, 104) sequence SEAfp18NT20: MPYSSESKEKETHSKETERD, or related CIS N-terminal signal peptides, as input sequence for a protein homology (above 60%) or pattern search using BLAST® (blastp suite, Quick BLASTP, PSI-BLAST, PHI-BLAST, DELTA-BLAST, http://blast.ncbi.nlm.nih.gov/Blast.cgi?PROGRAM=blastp&PAGE_TYPE=Bla stSearch&LINK_LOC=blasthome) we selected [1] Search for homologs of this peptide.

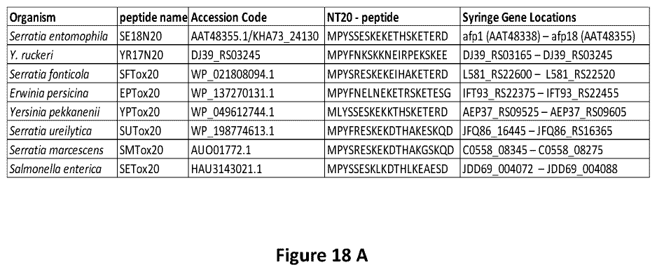

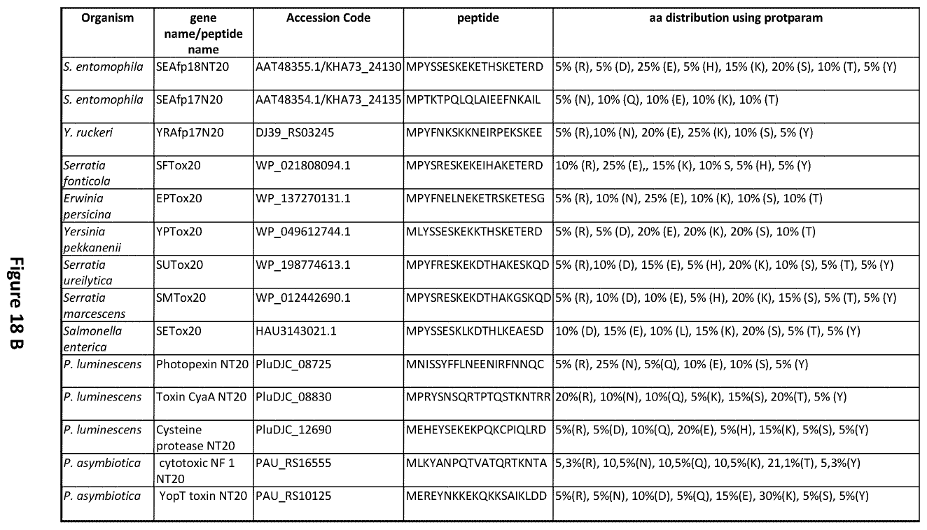

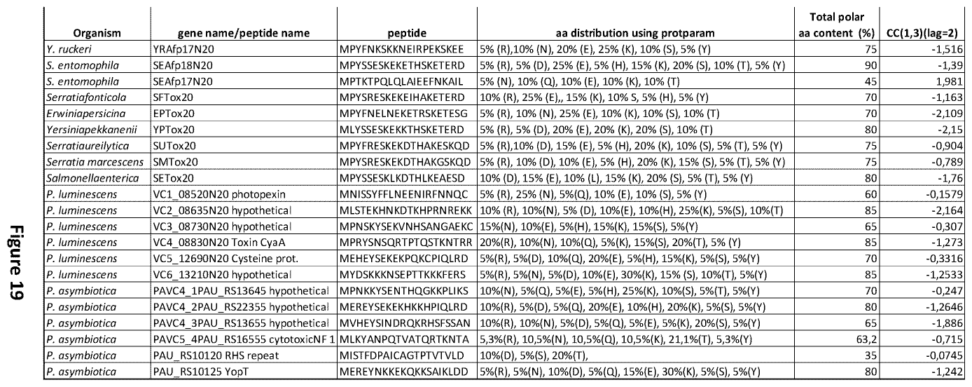

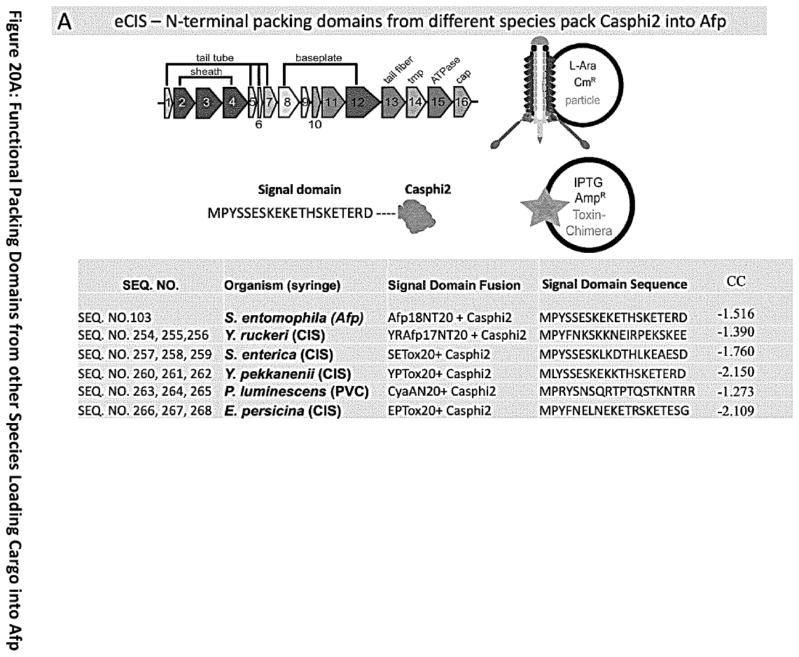

Manual investigation of each accession code (e.g. WP_049612744.1) for gene and genome location (NCBI nucleotide/genome database, www.ncbi.nlm.nih.gov) and validation of being next to homologous CIS syringe (https://www.ncbi.nlm.nih.gov/nuccore?LinkName=protein_nuccore_wp&from _uid=902545305). The homologous N-terminal packing motifs, accession codes, presence of CIS syringe are summarized in FIG. 18 (A). Any other protein databank or genome databank can be used for the homology search (e.g. https://www.genome.jp/, https://www.rcsb.org/, SWISS-PROT, dbeCIS: http://www.mgc.ac.cn/dbeCIS/etc.).

Raw output file: 79TBY202013-Alignment.txt (Where 79TBY202013 is Blastp JobID)

The result of the homology search of novel packing motifs and CIS syringes is shown in FIG. 18 (A).

We then identified packing motif parameters using alignment-independent cross covariance (CC) calculations and polar amino acid content.

The previous examples identified packing motifs of other organisms e.g., YRAfp17NT20 (SEQ. NO. 218, 254): MPYFNKSKKNEIRPEKSKEE (SEQ. NO. 218), and one packing motif that did not show successful packing SEAfp17NT20 (SEQ. NO. 173, 269): MPTKTPQLQLAIEEFNKAIL. Based on these results, we identified the packing motif parameter threshold.

Alignment based methods did not lead to a consensus sequence of signal peptides, but the sequences in FIG. 18 (A) obviously showed consensus content of high abundance of polar amino acids and similar electrochemical properties (FIG. 3(B) and 3(C)).

FIG. 3 (C) shows Multiple Sequence Alignment (ClustalOmega) of N-terminal signal peptides (SEQ. NO. 218-224, 87, 104) shown in FIG. 18 (A) and FIG. 3(B) high lightens the amino acid abundance as logo (WebLogo, https://weblogo.berkeley.edu/).

### Parameter 1: Alignment-Independent Cross Covariance (CC) Calculations

Since alignment-based methods, cannot account for gaps in motifs and disrupt the alignment to highlight similar motif properties (a consensus sequence), we highlighted parameters and qualification as packing motif using alignment-independent approaches, via cross covariance (CC) which is a transformation of peptide sequence into uniform vectors of principal amino acid properties described in z scales (Hellberg et al. 1987).

Two vectors, characterized in that said packing motif result in cross covariance (CC) lower than zero, where the first vector comprises the amino acid hydrophilicities (z1 scale) of each amino acid in said packing domain and second vector (z3 scale) comprising the electronic properties, represent the molecule's charge and polarity, having a lag of 2 for said first vector or said second vector.

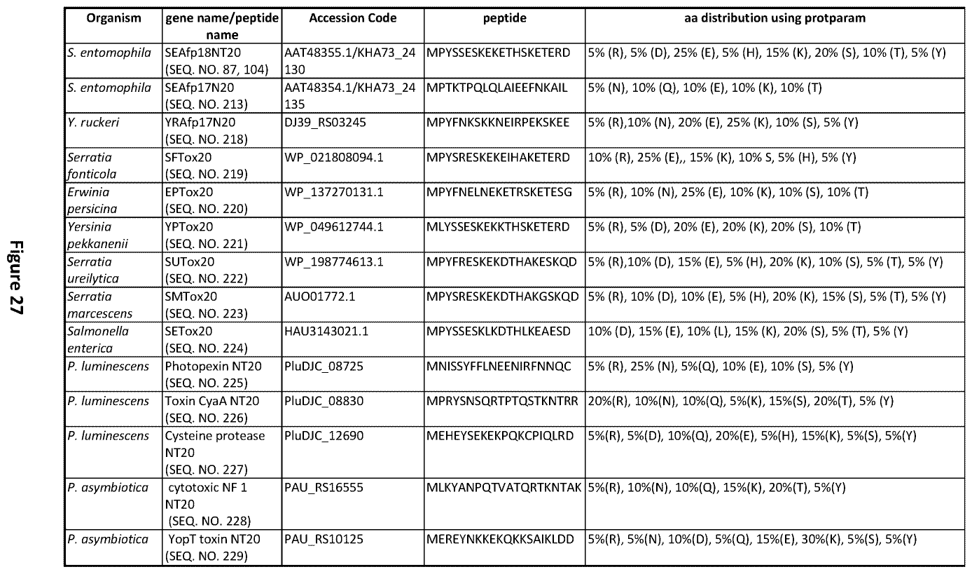

All packing motifs showed a CC (za≠zb,lag) (lag=2, z1 and z3-scale) value lower than zero and a validated non-packing motif showed a high positive value (FIG. 18 (B) and FIG. 27).

Packing motifs in novel and related CIS syringes and their negative CC1,3(lag=2) values are shown in FIG. 18 (B) and Firgure 27. SEAfp17N20 (SEQ. NO. 213) an experimentally confirmed motif that we showed did not pack successfully into the CIS of the invention has a high positive ACC1,3(lag=2) value. The CC values were calculated over the available VaxiJen server (http://www.ddg-pharmfac.net/vaxijenNaxiJenNaxiJen.html) which offers ACC and CC calculations of proteins. Total polar amino acid (aa) content was calculated using the ProtParam server (https://web.expasy.org/protparam/).

### Parameter 2: Polar Amino Acid Content

We found that packing motifs including more than 60% of polar amino acids, preferably with balanced and interspersed distribution of positive polar (lysine (K), histidine (H), arginine (R)) and negative polar (glutamic acid (E), (D)) supported by other polar amino acids asparagine (N), serine (S), threonine (T), glutamine (Q) provides an optimized sequence distribution to achieve high negative ACC1,3 (lag=2) values (FIG. 18 (B) and FIG. 27).

The program used here was ProtParam (https://web.expasy.org/protparam/) but any program or server that can analyze, calculate amino acid content, properties can be employed.

We also investigated known CIS syringe proteins located in a CVC cluster Known and identified CIS syringes and related secretion systems were analyzed with calculating CC1,3(lag=2) values and polar amino acid content as valuable parameters for potential packing motifs and packing ability.

We investigated the N-terminal packing domains for packing motifs of P. luminescens and Photorhabdus asymbiotica CIS syringes (FIG. 5 and FIG. 19).

We found that most of syringe related cargo showed qualified negative CC1,3(lag=2) values and more than 60% polar amino acid content.

Two examples that we had experimentally validated to not pack (SEAfp17N20 and PAAU_RS10120 RHS) (SEQ. NO.173, 168) were found to have lower than 50% polar amino acids and close to 0 but positive CC1,3(lag=2) values. The results are shown in FIG. 5, which is a representation of evolutionary related packing motifs and amino acid preferences/motifs using Seq2Logo, a web-based sequence logo generation method for construction and visualization of amino acid motifs (https://services.healthtech.dtu.dk/service.php?Seq2Logo-2.0). As can be seen in FIG. 5, Polar amino acids play an important role, particularly (lysine (K), glutamic acid (E), asparagine (N), arginine (R). FIG. 19 is a table disclosing the packing motif parameters for N-terminal peptides of CIS cargo.

We moreover tested the robustness of our identified characteristics of the packing motifs by generation of novel packing motifs.

Firstly, we randomized the amino acid sequence of known packing motifs and tested whether these sequences would still result in a CC below zero when applying a first vector comprising the amino acid hydrophilicities (z1 scale) of each amino acid in said packing domain and second vector (z3 scale) comprising the electronic properties, representing the molecule's charge and polarity, having a lag of 2 for said first vector or said second vector.

We found that changes in amino acid position of a confirmed packing motif does not drastically change its ability to serve as packing motif when looking at the CC1,3(lag=2). However, non-qualified motifs can be optimized to fulfill criteria CC1,3(lag=2) (see Table 3). An increase of polar amino acid above 60% and rearrangement of amino acid position can optimize a packing motif.

SE18N11 (SEQ. NO. 90, 107) ACC1,3(lag=2) values become positive (nonqualified) at 11 amino acids length, disqualifying the motif to serve for packing. This finding was confirmed by experimental data.

| gene name/peptide name | peptide | CC(1,3)lag = 2 |
|---|---|---|
| SEAfp18NT20 (SEQ. NO. 87, 104) | MPYSSESKEKETHSKETERD | −1.516 |
| SEAfp18N20backfront (SEQ. NO. 205) | MPYTHSKETERDSSESKEKE | −1.187 |
| SEAfp18N20reverse (SEQ. NO. 206) | MPYDRETEKSHTEKEKSESS | −0.617 |
| SEAfp18N201p1backforth (SEQ. NO. 207) | MDPRYESTSEEKSSEHETKE | −2.225 |
| SEAfp18N202p2forthback (SEQ. NO. 208) | MRDPYTESSKEESHSKETKE | −0.941 |
| SEAfp18N16 (SEQ. NO. 209) | MPYSSESKEKETHSKE | −0.0454 |
| SEAfp18N14 (SEQ. NO. 210) | MPYSSESKEKETHS | −0.462 |
| SEAfp18N12 (SEQ. NO. 211) | MPYSSESKEKET | −2.345 |
| SEAfp18NT11 (SEQ. NO. 90, 107) | MPYSSESKEKE | 0.028 |
| SEAfp18N9 (SEQ. NO. 212) | MPYSSESKE | 1.341 |
| SEAfp1720 (SEQ. NO. 213) | MPTKTPQLQLAIEEFNKAIL | 1.981 |
| SEAfp17N20backfront (SEQ. NO. 214) | MPIEEFNKAILTKTPQLQLA | 2.558 |
| SEAfp17N20reverse (SEQ. NO. 215) | MPLIAKNFEEIALQLQPTKT | 0.6589 |
| SEAfp17N201p1backforth (SEQ. NO. 216) | MLPITAKKTNPFQELEQILA | −0.1486 |
| SEAfp17N202p2forthback (SEQ. NO. 217) | MILPTKAKTFNPQEELQILA | −1.339 |

Table 3 shows the Randomized packing motifs (SEQ. NO. 205-217) and their CC1,3(lag=2) qualification parameter. Confirmed packing motifs (SEAafp18N20) (SEQ. NO.87, 104) can be rearranged and CC1,3(lag=2) value remained below zero. A packing motif (SEAfp17N20) (SEQ. NO.173, 213) that had been shown not to provide successful packaging of cargo into the syringe can be rearranged to improve CC1,3(lag=2) values.

The results found in Examples 1, 2, 3 and 4 altogether show that the packing motif is located within the N-terminal 20 amino acids of Afp18 and has specific characteristics, that can be found in other syringe related toxins and cargos (FIGS. 4 and 26). These characteristics are high hydrophilicity, and the electronic properties of the amino acids providing a CC below zero, moreover, the packing motif has a high abundance of polar amino acids >60%, (FIG. 17, FIGS. 4 and 26). The packing motif could attach two non-syringe related cargos (Casphi2, LL-37) to the syringe (FIG. 12). Packing motifs can be found in YRAfp17 which is a positive example for another packing motif (FIG. 17 (A) and 17 (B)) and is not present in SEAfp17 which cannot be attached to the syringe (FIG. 17 (A) and 17 (C)).

### Example 5—High Throughput Transport Assay and In Vivo Killing Assays Against Larvae

A high throughput transport assay is developed since working with alive larvae is very cumbersome and need careful handling and longer waiting times for confirming successful transport of effectors. This assay is made e.g., using CETSA (Martinez et al. 2018, a widely-applicable high-throughput cellular thermal shift assay (CETSA) using split Nano Luciferase, also coupled to mass spectrometry for detection of a substantial portion of the entire melting proteome of the target cell, organism or lysates thereof) or using superfolder fluorescence proteins as syringe cargo to be delivered into cells and/or larvae/larval lysates for confirmation of transport and validation of target cell. The results indicate that the test cargo is successfully transported over cell membranes.

### CROSS-REFERENCE TABLES

The below Table 4 lists the protein sequences disclosed herein by their SEQ. ID No., the name(s) applied in the description and Figures and sequence lists for the sequences, the key features of each sequence and further information about the sequence.

TABLE 4
PRT Sequence Table

| | Name of | | |
|---|---|---|---|
| | sequence | | |
| SEQ. | applied in | | |
| ID | Description | Key features corresponding to sequence | Information about the |
| NO. | and Figures | list | sequence |
| 2 | Afp1 | PRT transcript of SEQ. NO. 1: | transcript of CDS Afp1 of Afp |
| | | pBAD33_SE1-18 | syringe on SEQ. NO. 1: |
| | | | pBAD33_SE1-18 |
| 3 | Afp2 | PRT transcript of SEQ. NO. 1: | transcript of CDS Afp2 of Afp |
| | | pBAD33_SE1-18 | syringe on SEQ. NO. 1: |
| | | | pBAD33_SE1-18 |
| 4 | Afp3 | PRT transcript of SEQ. NO. 1: | transcript of CDS Afp3 of Afp |
| | | pBAD33_SE1-18 | syringe on SEQ. NO. 1: |
| | | | pBAD33_SE1-18 |
| 5 | Afp4 | PRT transcript of SEQ. NO. 1: | transcript of CDS Afp4 of Afp |
| | | pBAD33_SE1-18 | syringe on SEQ. NO. 1: |
| | | | pBAD33_SE1-18 |
| 6 | Afp5 | PRT transcript of SEQ. NO. 1: | transcript of CDS Afp5 of Afp |
| | | pBAD33_SE1-18 | syringe on SEQ. NO. 1: |
| | | | pBAD33_SE1-18 |
| 7 | Afp6 | PRT transcript of SEQ. NO. 1: | transcript of CDS Afp6 of Afp |
| | | pBAD33_SE1-18 | syringe on SEQ. NO. 1: |
| | | | pBAD33_SE1-18 |
| 8 | Afp7 | PRT transcript of SEQ. NO. 1: | transcript of CDS Afp7 of Afp |
| | | pBAD33_SE1-18 | syringe on SEQ. NO. 1: |
| | | | pBAD33_SE1-18 |
| 9 | Afp8 | PRT transcript of SEQ. NO. 1: | transcript of CDS Afp8 of Afp |
| | | pBAD33_SE1-18 | syringe on SEQ. NO. 1: |
| | | | pBAD33_SE1-18 |
| 10 | Afp9 | PRT transcript of SEQ. NO. 1: | transcript of CDS Afp9 of Afp |
| | | pBAD33_SE1-18 | syringe on SEQ. NO. 1: |
| | | | pBAD33_SE1-18 |
| 11 | Afp10 | PRT transcript of SEQ. NO. 1: | transcript of CDS Afp10 of Afp |
| | | pBAD33_SE1-18 | syringe on SEQ. NO. 1: |
| | | | pBAD33_SE1-18 |
| 12 | Afp11 | PRT transcript of SEQ. NO. 1: | transcript of CDS Afp11 of Afp |
| | | pBAD33_SE1-18 | syringe on SEQ. NO. 1: |
| | | | pBAD33_SE1-18 |
| 13 | Afp12 | PRT transcript of SEQ. NO. 1: | transcript of CDS Afp12 of Afp |
| | | pBAD33_SE1-18 | syringe on SEQ. NO. 1: |
| | | | pBAD33_SE1-18 |
| 14 | Afp13 | PRT transcript of SEQ. NO. 1: | transcript of CDS Afp13 of Afp |
| | | pBAD33_SE1-18 | syringe on SEQ. NO. 1: |
| | | | pBAD33_SE1-18 |
| 15 | Afp14 | PRT transcript of SEQ. NO. 1: | transcript of CDS Afp14 of Afp |
| | | pBAD33_SE1-18 | syringe on SEQ. NO. 1: |
| | | | pBAD33_SE1-18 |
| 16 | Afp15 | PRT transcript of SEQ. NO. 1: | transcript of CDS Afp15 of Afp |
| | | pBAD33_SE1-18 | syringe on SEQ. NO. 1: |
| | | | pBAD33_SE1-18 |
| 17 | Afp16 | PRT transcript of SEQ. NO. 1: | transcript of CDS Afp16 of Afp |
| | | pBAD33_SE1-18 | syringe on SEQ. NO. 1: |
| | | | pBAD33_SE1-18 |
| 18 | Afp17 | PRT transcript of SEQ. NO. 1: | transcript of CDS Afp17 of Afp |
| | | pBAD33_SE1-18 | syringe on SEQ. NO. 1: |
| | | | pBAD33_SE1-18 |
| 19 | Afp18 | PRT transcript of SEQ. NO. 1: | transcript of CDS Afp18 of Afp |
| | | pBAD33_SE1-18 | syringe on SEQ. NO. 1: |
| | | | pBAD33_SE1-18 |
| 21 | Afp1 | PRT transcript of SEQ. NO. 20: | transcript of CDS Afp1 of Afp |
| | | pBAD33_SE1-18C4 | syringe on SEQ. NO. 20: |
| | | | pBAD33_SE1-18C4 |
| 22 | Afp2 | PRT transcript of SEQ. NO. 20: | transcript of CDS Afp2 of Afp |
| | | pBAD33_SE1-18C4 | syringe on SEQ. NO. 20: |
| | | | pBAD33_SE1-18C4 |
| 23 | Afp3 | PRT transcript of SEQ. NO. 20: | transcript of CDS Afp3 of Afp |
| | | pBAD33_SE1-18C4 | syringe on SEQ. NO. 20: |
| | | | pBAD33_SE1-18C4 |
| 24 | Afp4 | PRT transcript of SEQ. NO. 20: | transcript of CDS Afp4 of Afp |
| | | pBAD33_SE1-18C4 | syringe on SEQ. NO. 20: |
| | | | pBAD33_SE1-18C4 |
| 25 | Afp5 | PRT transcript of SEQ. NO. 20: | transcript of CDS Afp5 of Afp |
| | | pBAD33_SE1-18C4 | syringe on SEQ. NO. 20: |
| | | | pBAD33_SE1-18C4 |
| 26 | Afp6 | PRT transcript of SEQ. NO. 20: | transcript of CDS Afp6 of Afp |
| | | pBAD33_SE1-18C4 | syringe on SEQ. NO. 20: |
| | | | pBAD33_SE1-18C4 |
| 27 | Afp7 | PRT transcript of SEQ. NO. 20: | transcript of CDS Afp7 of Afp |
| | | pBAD33_SE1-18C4 | syringe on SEQ. NO. 20: |
| | | | pBAD33_SE1-18C4 |
| 28 | Afp8 | PRT transcript of SEQ. NO. 20: | transcript of CDS Afp8 of Afp |
| | | pBAD33_SE1-18C4 | syringe on SEQ. NO. 20: |
| | | | pBAD33_SE1-18C4 |
| 29 | Afp9 | PRT transcript of SEQ. NO. 20: | transcript of CDS Afp9 of Afp |
| | | pBAD33_SE1-18C4 | syringe on SEQ. NO. 20: |
| | | | pBAD33_SE1-18C4 |
| 30 | Afp10 | PRT transcript of SEQ. NO. 20: | transcript of CDS Afp10 of Afp |
| | | pBAD33_SE1-18C4 | syringe on SEQ. NO. 20: |
| | | | pBAD33_SE1-18C4 |
| 31 | Afp11 | PRT transcript of SEQ. NO. 20: | transcript of CDS Afp11 of Afp |
| | | pBAD33_SE1-18C4 | syringe on SEQ. NO. 20: |
| | | | pBAD33_SE1-18C4 |
| 32 | Afp12 | PRT transcript of SEQ. NO. 20: | transcript of CDS Afp12 of Afp |
| | | pBAD33_SE1-18C4 | syringe on SEQ. NO. 20: |
| | | | pBAD33_SE1-18C4 |
| 33 | Afp13 | PRT transcript of SEQ. NO. 20: | transcript of CDS Afp13 of Afp |
| | | pBAD33_SE1-18C4 | syringe on SEQ. NO. 20: |
| | | | pBAD33_SE1-18C4 |
| 34 | Afp14 | PRT transcript of SEQ. NO. 20: | transcript of CDS Afp14 of Afp |
| | | pBAD33_SE1-18C4 | syringe on SEQ. NO. 20: |
| | | | pBAD33_SE1-18C4 |
| 35 | Afp15 | PRT transcript of SEQ. NO. 20: | transcript of CDS Afp15 of Afp |
| | | pBAD33_SE1-18C4 | syringe on SEQ. NO. 20: |
| | | | pBAD33_SE1-18C4 |
| 36 | Afp16 | PRT transcript of SEQ. NO. 20: | transcript of CDS Afp16 of Afp |
| | | pBAD33_SE1-18C4 | syringe on SEQ. NO. 20: |
| | | | pBAD33_SE1-18C4 |
| 37 | Afp17 | PRT transcript of SEQ. NO. 20: | transcript of CDS Afp17 of Afp |
| | | pBAD33_SE1-18C4 | syringe on SEQ. NO. 20: |
| | | | pBAD33_SE1-18C4 |
| 38 | Afp18C4 | PRT transcript of SEQ. NO. 20: | transcript of CDS Afp18 of Afp |
| | | pBAD33_SE1-18C4 | syringe on SEQ. NO. 20: |
| | | | pBAD33_SE1-18C4 |
| 40 | Afp1 | PRT transcript of SEQ. NO. 39: | transcript of CDS Afp1 of Afp |
| | | pBAD33_SE1-17 | syringe on SEQ. NO. 39: |
| | | | pBAD33_SE1-17 |
| 41 | Afp2 | PRT transcript of SEQ. NO. 39: | transcript of CDS Afp2 of Afp |
| | | pBAD33_SE1-17 | syringe on SEQ. NO. 39: |
| | | | pBAD33_SE1-17 |
| 42 | Afp3 | PRT transcript of SEQ. NO. 39: | transcript of CDS Afp3 of Afp |
| | | pBAD33_SE1-17 | syringe on SEQ. NO. 39: |
| | | | pBAD33_SE1-17 |
| 43 | Afp4 | PRT transcript of SEQ. NO. 39: | transcript of CDS Afp4 of Afp |
| | | pBAD33_SE1-17 | syringe on SEQ. NO. 39: |
| | | | pBAD33_SE1-17 |
| 44 | Afp5 | PRT transcript of SEQ. NO. 39: | transcript of CDS Afp5 of Afp |
| | | pBAD33_SE1-17 | syringe on SEQ. NO. 39: |
| | | | pBAD33_SE1-17 |
| 45 | Afp6 | PRT transcript of SEQ. NO. 39: | transcript of CDS Afp6 of Afp |
| | | pBAD33_SE1-17 | syringe on SEQ. NO. 39: |
| | | | pBAD33_SE1-17 |
| 46 | Afp7 | PRT transcript of SEQ. NO. 39: | transcript of CDS Afp7 of Afp |
| | | pBAD33_SE1-17 | syringe on SEQ. NO. 39: |
| | | | pBAD33_SE1-17 |
| 47 | Afp8 | PRT transcript of SEQ. NO. 39: | transcript of CDS Afp8 of Afp |
| | | pBAD33_SE1-17 | syringe on SEQ. NO. 39: |
| | | | pBAD33_SE1-17 |
| 48 | Afp9 | PRT transcript of SEQ. NO. 39: | transcript of CDS Afp9 of Afp |
| | | pBAD33_SE1-17 | syringe on SEQ. NO. 39: |
| | | | pBAD33_SE1-17 |
| 49 | Afp10 | PRT transcript of SEQ. NO. 39: | transcript of CDS Afp10 of Afp |
| | | pBAD33_SE1-17 | syringe on SEQ. NO. 39: |
| | | | pBAD33_SE1-17 |
| 50 | Afp11 | PRT transcript of SEQ. NO. 39: | transcript of CDS Afp11 of Afp |
| | | pBAD33_SE1-17 | syringe on SEQ. NO. 39: |
| | | | pBAD33_SE1-17 |
| 51 | Afp12 | PRT transcript of SEQ. NO. 39: | transcript of CDS Afp12 of Afp |
| | | pBAD33_SE1-17 | syringe on SEQ. NO. 39: |
| | | | pBAD33_SE1-17 |
| 52 | Afp13 | PRT transcript of SEQ. NO. 39: | transcript of CDS Afp13 of Afp |
| | | pBAD33_SE1-17 | syringe on SEQ. NO. 39: |
| | | | pBAD33_SE1-17 |
| 53 | Afp14 | PRT transcript of SEQ. NO. 39: | transcript of CDS Afp14 of Afp |
| | | pBAD33_SE1-17 | syringe on SEQ. NO. 39: |
| | | | pBAD33_SE1-17 |
| 54 | Afp15 | PRT transcript of SEQ. NO. 39: | transcript of CDS Afp15 of Afp |
| | | pBAD33_SE1-17 | syringe on SEQ. NO. 39: |
| | | | pBAD33_SE1-17 |
| 55 | Afp16 | PRT transcript of SEQ. NO. 39: | transcript of CDS Afp16 of Afp |
| | | pBAD33_SE1-17 | syringe on SEQ. NO. 39: |
| | | | pBAD33_SE1-17 |
| 56 | Afp17 | PRT transcript of SEQ. NO. 39: | transcript of CDS Afp17 of Afp |
| | | pBAD33_SE1-17 | syringe on SEQ. NO. 39: |
| | | | pBAD33_SE1-17 |
| 58 | Afp1 | PRT transcript of SEQ. NO. 57: | transcript of CDS Afp1 of Afp |
| | | pBAD33_SE1-16 | syringe on SEQ. NO. 57: |
| | | | pBAD33_SE1-16 |
| 59 | Afp2 | PRT transcript of SEQ. NO. 57: | transcript of CDS Afp2 of Afp |
| | | pBAD33_SE1-16 | syringe on SEQ. NO. 57: |
| | | | pBAD33_SE1-16 |
| 60 | Afp3 | PRT transcript of SEQ. NO. 57: | transcript of CDS Afp3 of Afp |
| | | pBAD33_SE1-16 | syringe on SEQ. NO. 57: |
| | | | pBAD33_SE1-16 |
| 61 | Afp4 | PRT transcript of SEQ. NO. 57: | transcript of CDS Afp4 of Afp |
| | | pBAD33_SE1-16 | syringe on SEQ. NO. 57: |
| | | | pBAD33_SE1-16 |
| 62 | Afp5 | PRT transcript of SEQ. NO. 57: | transcript of CDS Afp5 of Afp |
| | | pBAD33_SE1-16 | syringe on SEQ. NO. 57: |
| | | | pBAD33_SE1-16 |
| 63 | Afp6 | PRT transcript of SEQ. NO. 57: | transcript of CDS Afp6 of Afp |
| | | pBAD33_SE1-16 | syringe on SEQ. NO. 57: |
| | | | pBAD33_SE1-16 |
| 64 | Afp7 | PRT transcript of SEQ. NO. 57: | transcript of CDS Afp7 of Afp |
| | | pBAD33_SE1-16 | syringe on SEQ. NO. 57: |
| | | | pBAD33_SE1-16 |
| 65 | Afp8 | PRT transcript of SEQ. NO. 57: | transcript of CDS Afp8 of Afp |
| | | pBAD33_SE1-16 | syringe on SEQ. NO. 57: |
| | | | pBAD33_SE1-16 |
| 66 | Afp9 | PRT transcript of SEQ. NO. 57: | transcript of CDS Afp9 of Afp |
| | | pBAD33_SE1-16 | syringe on SEQ. NO. 57: |
| | | | pBAD33_SE1-16 |
| 67 | Afp10 | PRT transcript of SEQ. NO. 57: | transcript of CDS Afp10 of Afp |
| | | pBAD33_SE1-16 | syringe on SEQ. NO. 57: |
| | | | pBAD33_SE1-16 |
| 68 | Afp11 | PRT transcript of SEQ. NO. 57: | transcript of CDS Afp11 of Afp |
| | | pBAD33_SE1-16 | syringe on SEQ. NO. 57: |
| | | | pBAD33_SE1-16 |
| 69 | Afp12 | PRT transcript of SEQ. NO. 57: | transcript of CDS Afp12 of Afp |
| | | pBAD33_SE1-16 | syringe on SEQ. NO. 57: |
| | | | pBAD33_SE1-16 |
| 70 | Afp13 | PRT transcript of SEQ. NO. 57: | transcript of CDS Afp13 of Afp |
| | | pBAD33_SE1-16 | syringe on SEQ. NO. 57: |
| | | | pBAD33_SE1-16 |
| 71 | Afp14 | PRT transcript of SEQ. NO. 57: | transcript of CDS Afp14 of Afp |
| | | pBAD33_SE1-16 | syringe on SEQ. NO. 57: |
| | | | pBAD33_SE1-16 |
| 72 | Afp15 | PRT transcript of SEQ. NO. 57: | transcript of CDS Afp15 of Afp |
| | | pBAD33_SE1-16 | syringe on SEQ. NO. 57: |
| | | | pBAD33_SE1-16 |
| 73 | Afp16 | PRT transcript of SEQ. NO. 57: | transcript of CDS Afp16 of Afp |
| | | pBAD33_SE1-16 | syringe on SEQ. NO. 57: |
| | | | pBAD33_SE1-16 |
| 75 | Afp18, | PRT transcript of SEQ. NO. 74: | transcript of CDS Afp18 toxin on |
| | SEAfp18 | pET11a_SEAfp18 | SEQ. NO. 74: pET11a_SEAfp18 |
| 77 | YRAfp17, | PRT transcript of SEQ. NO. 76: | transcript of CDS YRAfp17 toxin |
| | YR17 | pET11a_YRAfp17 | on SEQ. NO. 76: |
| | | | pET11a_YRAfp17 |
| 79 | Photopexin | PRT transcript of SEQ. NO. 78: | transcript of CDS of Photopexin |
| | | pET11a_Photopexin_PLUDJC_08520 | PluDJC_08520 SEQ. NO. 78: |
| | | | pET11a_ |
| | | | Photopexin_PLUDJC_08520, |
| | | | Photorhabdus luminescens |
| | | | photopexin |
| 81 | SEAfp18NT50 | PRT transcript of SEAfp18NT50 from | transcript of CDS SEAfp18NT50 |
| | Afp18NT50, | SEQ. NO. 80: | on SEQ. NO. 80: |
| | 18NT50, NT50 | pET11a_SEAfp18-NT50-LL37 | pET11a_SEAfp18-NT50-LL37 |
| 82 | LL37 | PRT transcript of LL37 from SEQ. NO. | transcript of CDS LL37 on SEQ. |
| | | 80: | NO. 80: |
| | | pET11a_SEAfp18-NT50-LL37 | pET11a_SEAfp18-NT50-LL37 |
| 84 | SEAfp18NT30 | PRT transcript of SEAfp18NT30 from | transcript of CDS SEAfp18NT30 |
| | Afp18NT30, | SEQ. NO. 83: | on SEQ. NO. 83: |
| | 18NT30, NT30 | pET11a_SEAfp18-NT30-LL37 | pET11a_SEAfp18-NT30-LL37 |
| 85 | LL37 | PRT transcript of LL37 from SEQ. NO. | transcript of CDS LL37 on SEQ. |
| | | 83: | NO. 83: |
| | | pET11a_SEAfp18-NT30-LL37 | pET11a_SEAfp18-NT30-LL37 |
| 87 | SEAfp18NT20 | PRT transcript of SEAfp18NT20 from | transcript of CDS SEAfp18NT20 |
| | Afp18NT20, | SEQ. NO. 86: | on SEQ. NO. 86: |
| | 18NT20, NT20 | pET11a_Afp18NT20-LL37 | pET11a_Afp18NT20- LL37 |
| 88 | LL37 | PRT transcript of LL37 from SEQ. NO. | transcript of CDS LL37 on SEQ. |
| | | 86: | NO. 86: |
| | | pET11a_Afp18NT20- LL37 | pET11a_Afp18NT20-LL37 |
| 90 | SEAfp18NT11,, | PRT transcript of SEAfp18NT11 from | transcript of CDS SEAfp18NT11 |
| | Afp18NT11, | SEQ. NO. 89: | on SEQ. NO. 89: |
| | 18NT11, NT11 | pET11a_SEAfp18-NT11-LL37 | pET11a_SEAfp18-NT11-LL37 |
| 91 | LL37 | PRT transcript of LL37 from SEQ. NO. | transcript of CDS LL37 on SEQ. |
| | | 89: | NO. 89: |
| | | pET11a_SEAfp18-NT11-LL37 | pET11a_SEAfp18-NT11-LL37 |
| 93 | SEAfp18NT5, | PRT transcript of SEAfp18NT5 from SEQ. | transcript of CDS SEAfp18NT5 |
| | Afp18NT5, | NO. 92: | on SEQ. NO. 92: |
| | 18NT5, NT5 | pET11a_SEAfp18-NT5-LL37 | pET11a_SEAfp18-NT5-LL37 |
| 94 | LL37 | PRT transcript of LL37 from SEQ. NO. | transcript of CDS LL37 on SEQ. |
| | | 92: | NO. 92: |
| | | pET11a_SEAfp18-NT5-LL37 | pET11a_SEAfp18-NT5-LL37 |
| 96 | LL37 | PRT transcript of LL37 from SEQ. NO. | transcript of CDS LL37 on SEQ. |
| | | 95: | NO. 95: |
| | | pET11a_SEAfp18-NT2-LL37 | pET11a_SEAfp18-NT2-LL37 |
| 98 | SEAfp18NT50, | PRT transcript of SEAfp18NT50 from | transcript of CDS SEAfp18NT50 |
| | Afp18NT50, | SEQ. NO. 97: | on SEQ. NO. 97: |
| | 18NT50, NT50 | pET11a_SEAfp18-NT50-Casphi2 | pET11a_SEAfp18-NT50- |
| | | | Casphi2 |
| 99 | Casphi2 | PRT transcript of Casphi2 from SEQ. | transcript of CDS Casphi2 on |
| | | NO. 97: | SEQ. NO. 97: |
| | | pET11a_SEAfp18-NT50-Casphi2 | pET11a_SEAfp18-NT50- |
| | | | Casphi2 |
| 101 | SEAfp18NT30, | PRT transcript of SEAfp18NT30 from | transcript of CDS SEAfp18NT30 |
| | Afp18NT30, | SEQ. NO. 100: | on SEQ. NO. 100: |
| | 18NT30, NT30 | pET11a_SEAfp18-NT30-Casphi2 | pET11a_SEAfp18-NT30- |
| | | | Casphi2 |
| 102 | Casphi2 | PRT transcript of Casphi2 from SEQ. | transcript of CDS Casphi2 on |
| | | NO. 100: | SEQ. NO. 100: |
| | | pET11a_SEAfp18-NT30-Casphi2 | pET11a_SEAfp18-NT30- |
| | | | Casphi2 |
| 104 | SEAfp18NT20, | PRT transcript of SEAfp18NT20 from | transcript of CDS SEAfp18NT20 |
| | Afp18NT20, | SEQ. NO. 103: | on SEQ. NO. 103: |
| | 18NT20, NT20 | pET11a_Afp18NT20-Casphi2 | pET11a_Afp18NT20-Casphi2 |
| 105 | Casphi2 | PRT transcript of Casphi2 from SEQ. | transcript of CDS Casphi2 on |
| | | NO. 103: | SEQ. NO. 103: |
| | | pET11a_Afp18NT20-Casphi2 | pET11a_Afp18NT20-Casphi2 |
| 107 | SEAfp18NT11, | PRT transcript of SEAfp18NT11 from | transcript of CDS SEAfp18NT11 |
| | Afp18NT11, | SEQ. NO. 106: | on SEQ. NO. 106: |
| | 18NT11, NT11 | pET11a_SEAfp18-NT11-Casphi2 | pET11a_SEAfp18-NT11- |
| | | | Casphi2 |
| 108 | Casphi2 | PRT transcript of Casphi2 from SEQ. | transcript of CDS Casphi2 on |
| | | NO. 106: | SEQ. NO. 106: |
| | | pET11a_SEAfp18-NT11-Casphi2 | pET11a_SEAfp18-NT11- |
| | | | Casphi2 |
| 110 | SEAfp18NT5, | PRT transcript of SEAfp18NT5 from SEQ. | transcript of CDS SEAfp18NT5 |
| | Afp18NT5, | NO. 109: | on SEQ. NO. 109: |
| | 18NT5, NT5 | pET11a_SEAfp18-NT5-Casphi2 | pET11a_SEAfp18-NT5-Casphi2 |
| 111 | Casphi2 | PRT transcript of Casphi2 from SEQ. | transcript of CDS Casphi2 on |
| | | NO. 109: | SEQ. NO. 109: |
| | | pET11a_SEAfp18-NT5-Casphi2 | pET11a_SEAfp18-NT5-Casphi2 |
| 113 | Casphi2 | PRT transcript of Casphi2 from SEQ. | transcript of CDS Casphi2 on |
| | | NO. 112: | SEQ. NO. 112: |
| | | pET11a_SEAfp18-NT2-Casphi2 | pET11a_SEAfp18-NT2-Casphi2 |
| 115 | SEAfp18-C1, | PRT transcript of SEAfp18-C1 from SEQ. | transcript of CDS SEAfp18-C1 |
| | C1 | NO. 114: | Afp18 C terminal truncation at |
| | | pET11a_SEAfp18-C1 | L2181 |
| 117 | SEAfp18-C2, | PRT transcript of SEAfp18-C2 from SEQ. | transcript of CDS SEAfp18-C2 |
| | C2 | NO. 116: | Afp18 C-terminal truncation at |
| | | pET11a_SEAfp18-C2 | A1863 |
| 119 | SEAfp18-C3, | PRT transcript of SEAfp18-C3 from SEQ. | transcript of CDS SEAfp18-C3 |
| | C3 | NO. 118: | Afp18 C-terminal truncation |
| | | pET11a_SEAfp18-C3 | E1694 |
| 121 | SEAfp18-C4, | PRT transcript of SEAfp18-C4 from SEQ. | transcript of CDS SEAfp18-C4 |
| | C4 | NO. 120: | Afp18 C-terminal truncation |
| | | pET11a_SEAfp18-C4 | 11437 |
| 123 | SEAfp18-C6, | PRT transcript of SEAfp18-C6 from SEQ. | transcript of CDS SEAfp18-C6 |
| | C6 | NO. 122: | Afp18 C-terminal truncation |
| | | pET11a_SEAfp18-C6 | K526 |
| 125 | SEAfp18-N7, | PRT transcript of SEAfp18-N7 from SEQ. | transcript of CDS SEAfp18-N7 |
| | N7 | NO. 124: | Afp18 N-terminal truncation (M) |
| | | pET11a_SEAfp18-N7 | 2182 |
| 127 | SEAfp18-N6, | PRT transcript of SEAfp18-N6 from SEQ. | transcript of CDS SEAfp18-N6 |
| | N6 | NO. 126: | Afp18 N-terminal truncation (M) |
| | | pET11a_SEAfp18-N6 | 1864 |
| 129 | SEAfp18-N5, | PRT transcript of SEAfp18-N5 from SEQ. | transcript of CDS SEAfp18-N5 |
| | N5 | NO. 128: | Afp18 N-terminal truncation (M) |
| | | pET11a_SEAfp18-N5 | 1695 |
| 131 | SEAfp18-N4, | PRT transcript of SEAfp18-N4 from SEQ. | transcript of CDS SEAfp18-N4 |
| | N4 | NO. 130: | Afp18 N-terminal truncation (M) |
| | | pET11a_SEAfp18-N4 | 1438 |
| 133 | SEAfp18-N2, | PRT transcript of SEAfp18-N2 from SEQ. | transcript of CDS SEAfp18-N2 |
| | N2 | NO. 132: | Afp18 N-terminal truncation (M) |
| | | pET11a_SEAfp18-N2 | 527 |
| 135 | SEAfp18-N1, | PRT transcript of SEAfp18-N1 from SEQ. | transcript of CDS SEAfp18-N1 |
| | N1 | NO. 134: | Afp18 N-terminal truncation |
| | | pET11a_SEAfp18-N1 | M221 |
| 137 | SEAfp18-NX1, | PRT transcript of SEAfp18-NX1 from SEQ. | transcript of CDS SEAfp18-NX1 |
| | NX1 | NO. 136: | Afp18 N-terminal truncation |
| | | pET11a_SEAfp18-NX1 | M172 |
| 139 | SEAfp18-NX2, | PRT transcript of SEAfp18-NX2 from SEQ. | transcript of CDS SEAfp18-NX2 |
| | NX2 | NO. 138: | Afp18 N-terminal truncation A96 |
| | | pET11a_SEAfp18-NX2 | and insertion of a start |
| | | | Methionine |
| 141 | SEAfp18-NX3, | PRT transcript of SEAfp18-NX3 from SEQ. | transcript of CDS SEAfp18-NX3 |
| | NX3 | NO. 140: | Afp18 N-terminal truncation S30 |
| | | pET11a_SEAfp18-NX3 | and addition of start Methionine |
| 143 | SEAfp18-C10, | PRT transcript of SEAfp18-C10 from SEQ. | transcript of CDS SEAfp18-C10 |
| | C10 | NO. 142: | Afp18 C10 terminal truncation |
| | | pET11a_SEAfp18-C10 | S30 |
| 145 | SEAfp18-C9, | PRT transcript of SEAfp18-C9 from SEQ. | transcript of CDS SEAfp18-C9 |
| | C9 | NO. 144: | Afp 18 C terminal truncation |
| | | pET11a_SEAfp18-C9 | A95 |
| 147 | SEAfp18-C8, | PRT transcript of SEAfp18-C8 from SEQ. | transcript of CDS SEAfp18-C8 |
| | C8 | NO. 146: | Afp18 C8 truncation T171 |
| | | pET11a_SEAfp18-C8 | |
| 149 | SEAfp18-C7, | PRT transcript of SEAfp18-C7 from SEQ. | transcript of CDS SEAfp18-C7 |
| | C7 | NO. 148: | Afp18 C7 terminal truncation |
| | | pET11a_SEAfp18-C7 | R220 |
| 151 | Afp18C8, | PRT transcript of SEAfp18-C8 from SEQ. | transcript of CDS SEAfp18-C8 |
| | 18C8, C8 | NO. 150: | Afp18-C8 C-terminal truncation |
| | | pET11a_ Afp18C8-Casphi2 | T171 |
| 152 | Casphi2 | PRT transcript of Capshi from SEQ. | Casphi2 transcript of CDS |
| | | NO. 150: | Casphi2 codon optimized |
| | | pET11a_ Afp18C8-Casphi2 | |
| 154 | Afp18C8, | PRT transcript of SEAfp18-C8 from SEQ. | transcript of CDS SEAfp18-C8 |
| | 18C8, C8 | NO. 153: | Afp18-C8 C-terminal truncation |
| | | pET11a_ Afp18C8-PAExoU | T171 |
| 155 | PAExoU, ExoU | PRT transcript of PAExoU from SEQ. | transcript of CDS Pseudomonas |
| | | NO. 153: | aeruginosa Type III Secrection |
| | | pET11a_Afp18C8-PAExoU | System Effector ExoU |
| 157 | Afp18C8, | PRT transcript of SEAfp18-C8 from SEQ. | transcript of CDS SEAfp18-C8 |
| | 18C8, C8 | NO. 156: | Afp18-C8 C-terminal truncation |
| | | pET11a_ Afp18C8-PATse1 | T171 |
| 158 | PATse1 | PRT transcript of PATse1 from SEQ. | transcript of CDS Pseudomonas |
| | | NO. 156: | aeruginosa Type VI Secretion |
| | | pET11a_ Afp18C8-PATse1 | System Effector Tse1 |
| 160 | Afp18C8, | PRT transcript of SEAfp18-C8 from SEQ. | transcript of CDS SEAfp18-C8 |
| | 18C8, C8 | NO. 159: | Afp18-C8 C-terminal truncation |
| | | pET11a_Afp18C8-LL37 | T171 |
| 161 | LL37 | PRT transcript of LL37 from SEQ. | transcript of CDS LL37 human |
| | | NO. 159: | antimicrobial peptide, codon |
| | | pET11a_Afp18C8-LL37 | optimized for bacterial |
| | | | expression |
| 163 | Afp18C6, | PRT transcript of SEAfp18-C6 from SEQ. | transcript of CDS SEAfp18-C6 |
| | 18C6, C6 | NO. 162: | Afp18 C-terminal truncation |
| | | pET11a_Afp18C6-Cas9 | K526 |
| 164 | Cas9 | PRT transcript of Cass9 from SEQ. | transcript of CDS Cas9 from |
| | | NO. 162: | Francisella novicida |
| | | pET11a_Afp18C6-Cas9 | |
| 166 | Afp18C10, | PRT transcript of SEAfp18-C10 from SEQ. | transcript of CDS SEAfp18-C10 |
| | C10 | NO. 165: | Afp18 C10 terminal truncation |
| | | pET11a_Afp18C10-PAYopT-RHS_PAU_RS10125 | S30 |
| 167 | PAYopT_PAU_ | PRT transcript of PAYopT_PAU_RS10125 | transcripts of CDS |
| | RS10125, | from SEQ. NO. 165: | Photorhabdus asymbiotica YopT |
| | PAYopT | pET11a_Afp18C10-PAYopT-RHS_PAU_RS10125 | (PAU_RS10125) |
| 168 | PAYopT_PAU_ | PRT transcript of PARHS_PAU_RS10120 | transcripts of CDS |
| | RS10120, | from SEQ. NO. 165: | Photorhabdus asymbiotica RHS |
| | PARHS | pET11a_Afp18C10-PAYopT-RHS_PAU_RS10125 | repeat containing protein |
| | | | (PAU_RS10120) |
| 170 | Afp18C8, | PRT transcript of SEAfp18-C8 from SEQ. | transcript of CDS SEAfp18-C8 |
| | 18C8, C8 | NO. 169: | Afp18-C8 C-terminal truncation |
| | | pET11a_Afp18C8-PLRtxA toxinPluDJC_12685 | T171 |
| 171 | PLRtxA toxin | PRT transcript of Photorhabdus | transcript of CDS Photorhabdus |
| | PluDJC_12685, | luminescens | luminescens RtxA toxin |
| | PLRtxA | RtxA toxin (PluDJC_12685) SEQ. NO. 169: | (PluDJC_12685) |
| | | pET11a_Afp18C8-PLRtxA toxin | |
| | | PluDJC_12685 | |
| 173 | Afp17 | PRT transcript of SEAfp17 from SEQ. NO. | transcript of CDS SEAfp17 |
| | | 172: | remnant toxin |
| | | pET11a_Afp17-Afp18 | |
| 174 | Afp18 | PRT transcript of SEAfp18 from SEQ. NO. | transcript of CDS SEAfp18 toxin |
| | | 172: | |
| | | pET11a_Afp17-Afp18 | |
| 176 | Afp18, 18 | PRT transcript of SEAfp18 from SEQ. NO. | transcript of CDS Afp18 toxin |
| | | 175: | |
| | | pET11a_Afp18-sfGFP | |
| 177 | sfGFP | PRT transcript of sfGFP from SEQ. NO. | transcript of CDS superfolder |
| | | 175: | GFP (sfGFP) |
| | | pET11a_Afp18-sfGFP | |
| 179 | Afp18 | PRT transcript of SEAfp18 from SEQ. | transcript of CDS Afp18 toxin |
| | | NO .: 178 | |
| 180 | C3CTS | PRT transcript of C-terminal Twin | transcript of CDS C-terminal |
| | | Strep Tag from SEQ. NO .: 178 | Twin Strep Tag |
| 182 | Afp18C6 | PRT transcript of SEAfp18-C6 from SEQ. | transcript of CDS SEAfp18-C6 |
| | | NO. 181: | Afp18 C-terminal truncation |
| | | pET11a_Afp18C6-sfGFP | K526 |
| 183 | sfGFP | PRT transcript of sfGFP from SEQ. NO. | transcript of CDS superfolder |
| | | 181: | GFP (sfGFP) |
| | | pET11a_Afp18C6-sfGFP | |
| 184 | Mxe GyrA | PRT transcript of Mxe GyrA intein from | intein Mxe Gyra |
| | intein | SEQ. NO. 181: | |
| | | pET11a_Afp18C6-sfGFP | |
| 185 | CBD | PRT transcript of CBD from SEQ. NO. | C-terminal binding domain |
| | | 181: | (CBD) |
| | | pET11a_Afp18C6-sfGFP | |
| 187 | Afp18C4, | PRT transcript of SEAfp18-C4 from SEQ. | transcript of CDS SEAfp18-C4 |
| | 18C4 | NO. 186: | Afp18 C-terminal truncation |
| | | pET11a_Afp18C4-sfGFP | 11437 |
| 188 | sfGFP | PRT transcript of sfGFP from SEQ. NO. | transcript of CDS superfolder |
| | | 186: | GFP (sfGFP) |
| | | pET11a_Afp18C4-sfGFP | |
| 189 | Mxe GyrA | PRT transcript of Mxe GyrA intein from | intein Mxe Gyra |
| | intein | SEQ. NO. 186: | |
| | | pET11a_Afp18C4-sfGFP | |
| 190 | CBD | PRT transcript of CBD from SEQ. NO. | C-terminal binding domain |
| | | 186: | (CBD) |
| | | pET11a_Afp18C4-sfGFP | |
| 192 | Afp18C4, 18C4 | PRT transcript of SEAfp18-C4 from SEQ. | transcript of CDS SEAfp18-C4 |
| | | NO. 191: | Afp18 C-terminal truncation |
| | | pET11a_Afp18C4-Photopexin PluDJC_08520 | 11437 |
| 193 | Photopexin | PRT transcript of Photorhabdus | transcript of CDS Photorhabdus |
| | | luminescens | luminescens Photopexin |
| | | Photopexin PluDJC_08520 SEQ. NO. 191: | PluDJC_08520 |
| | | pET11a_Afp18C4-Photopexin | |
| | | PluDJC_08520 | |
| 195 | Afp18C6, | PRT transcript of SEAfp18-C6 from SEQ. | transcript of CDS SEAfp18-C6 |
| | 18C6, C6 | NO. 194: | Afp18 C-terminal truncation |
| | | pET11a_Afp18C6-YRAfp17 | K526 |
| 196 | YRAfp17, | PRT transcript of Yersinia ruckeri | transcript of CDS Yersinia |
| | YR17 | Afp17 (502-2123 amino acids, C6-YR) | ruckeri Afp17 (502-2123 amino |
| | | SEQ. NO. 194: | acids, C6-YR) |
| | | pET11a_Afp18C6-YRAfp17 | |
| 198 | Afp18C4, | PRT transcript of SEAfp18-C4 from SEQ. | transcript of CDS SEAfp18-C4 |
| | 18C4 | NO. 197: | Afp18 C-terminal truncation |
| | | pET11a_Afp18C4-YRAfp17 | 11437 |
| 199 | YRAfp17, | PRT transcript of Yersinia ruckeri | transcript of Yersinia ruckeri |
| | YR17 | Afp17 | Afp17 (1437-2123, amino acids, |
| | | (1437-2123, amino acids, C4-YR) | C4-YR) |
| 200 | SEAfp18, | BINDING-SE_Afp18-ag1 (4)..(12) | transcript of CDS SEAfp18 and |
| | Antibody | SE_Afp18-ag1 SSESKEKETHSKET | antibodies designed to bind |
| | SEAfp18, | MISC_FEATURE (4)..(12) | Afp18 toxin (Afp18-ag1-6) |
| | Afp18-ag1-6 | BINDING-SE_Afp18-ag2 (939)..(953) | |
| | | SE_Afp18-ag2 RVRKRAVADMAPPPP | |
| | | MISC_FEATURE (939)..(953) | |
| | | BINDING-SE_Afp18-ag3 (1463)..(1476) | |
| | | SE_Afp18-ag3 SSKFKSSDEFKVDK | |
| | | MISC_FEATURE (1463)..(1476) | |
| | | BINDING-SE_Afp18-ag4 (1741)..(1754) | |
| | | SE_Afp18-ag4 IIESKNSLDHDTSY | |
| | | MISC_FEATURE (1741)..(1754) SE_Afp18- | |
| | | ag4 IIESKNSLDHDTSY | |
| | | BINDING-SE_Afp18-ag5 (1921)..(1936) | |
| | | SE_Afp18-ag5 PPTRDSRYFYENER | |
| | | MISC_FEATURE (1921)..(1936) SE_Afp18- | |
| | | ag5 PPTRDSRYFYENER | |
| | | BINDING-SE_Afp18-ag6 (2306)..(2318) | |
| | | SE_Afp18-ag6 NYKVREKDNLEKK | |
| | | MISC_FEATURE (2306)..(2318) SE_Afp18- | |
| | | ag6 NYKVREKDNLEKK | |
| 201 | SEAfp17, | BINDING-SE_Afp17-ag1 (104)..(117) | transcript of CDS SEAfp17 and |
| | Antibody | SE_Afp17-ag1 DPSFTITKTNQDAG | antibodies designed to bind |
| | SEAfp17, | MISC_FEATURE (104)..(117) SE_Afp17-ag1 | Afp17 remnant toxin (Afp17- |
| | Afp17-ag1-2 | DPSFTITKTNQDAG | ag1&2) |
| | | BINDING-SE_Afp17-ag2 (267)..(280) | |
| | | SE_Afp17-ag2 SFSLRERDEHESGY | |
| | | MISC_FEATURE (267)..(280) SE_Afp17- | |
| | | ag2 SFSLRERDEHESGY | |
| 202 | SEAfp2, | BINDING-SE_Afp2-ag1 (5)..(18) SE_Afp2- | transcript of CDS SEAfp2 and |
| | Afp2ag, | ag1 TTYPGVYLSEDAVS | antibodies designed to bind |
| | antibody | MISC_FEATURE (5)..(18) SE_Afp2-ag1 | Afp2 |
| | syringe | TTYPGVYLSEDAVS | |
| 203 | SEAfp3, | BINDING-SE_Afp3-ag1 (61)..(75) SE_Afp3- | transcript of CDS SEAfp3 and |
| | Afp3ag, | ag1 DSNVPSSARVTVSST | antibodies designed to bind |
| | antibody | MISC_FEATURE (61)..(75) SE_Afp3-ag1 | Afp3 |
| | syringe | DSNVPSSARVTVSST | |
| 204 | YRAfp17, | BINDING-YR_Afp17_ag1 (2105)..(2119) | transcript of CDS YRAfp17 and |
| | YR17ag | YR_Afp17_ag1 DHSWVTDMPGNSTQS | antibodies designed to bind Y. |
| | | MISC_FEATURE (2105)..(2119) | ruckeri Afp17 |
| | | YR_Afp17_ag1 DHSWVTDMPGNSTQS | |
| | | PEPTIDE (1)..(20) | |
| 205 | SEAfp18N20- | MPYTHSKETERDSSESKEKE | Randomized N-terminal peptide |
| | backfront | SEAfp18N20backfront randomized version | of SEAfp18 (SEQ. NO. 19) |
| | | of SEQ. NO. 87, 104 | |
| 206 | SEAfp18N20 | PEPTIDE (1)..(20) | Randomized N-terminal peptide |
| | reverse | MPYDRETEKSHTEKEKSESS | of SEAfp18 (SEQ. NO. 19) |
| | | SEAfp18N20 reverse, randomized version | |
| | | of SEQ. NO. 87, 104 | |
| 207 | SEAfp18N2010 | PEPTIDE (1)..(20) | Randomized N-terminal peptide |
| | p1backforth | MDPRYESTSEEKSSEHETKE | of SEAfp18 (SEQ. NO. 19) |
| | | SEAfp18N201p1backforth, randomized | |
| | | version of SEQ. NO. 87, 104 | |
| 208 | SEAfp18N202- | PEPTIDE (1)..(20) | Randomized N-terminal peptide |
| | p2forthback | MRDPYTESSKEESHSKETKE | of SEAfp18 (SEQ. NO. 19) |
| | | SEAfp18N202p2forthback, randomized | |
| | | version of SEQ. NO. 87, 104 | |
| 209 | SEAfp18N16 | PEPTIDE (1)..(16) | Truncated N-terminal peptide of |
| | | MPYSSESKEKETHSKE SEAfp18N16, | SEAfp18 (SEQ. NO. 19) |
| | | truncated version of SEAfp18NT20 | |
| | | (SEQ. NO. 87, 104) | |
| 210 | SEAfp18N14 | PEPTIDE (1)..(14) | Truncated N-terminal peptide of |
| | | MPYSSESKEKETHS SEAfp18N14, | SEAfp18 (SEQ. NO. 19) |
| | | truncated version of SEAfp18NT20 (SEQ. | |
| | | NO. 87, 104) | |
| 211 | SEAfp18N12 | PEPTIDE (1)..(12) | Truncated N-terminal peptide of |
| | | MPYSSESKEKET SEAfp18N12, truncated | SEAfp18 (SEQ. NO. 19) |
| | | version of SEAfp18NT20 (SEQ. NO. 87, | |
| | | 104) | |
| 212 | SEAfp18N9 | PEPTIDE (1)..(9) | Truncated N-terminal peptide of |
| | | MPYSSESKE SEAfp18N9, truncated version | SEAfp18 (SEQ. NO. 19) |
| | | of SEAfp18NT20 (SEQ. NO. 87, 104) and | |
| | | of SEAfp18NT11 (SEQ. NO. 90, 107) | |
| 213 | SEAfp17N20 | PEPTIDE (1)..(20) | Truncated N-terminal peptide of |
| | SE17N20 | MPTKTPQLQLAIEEFNKAIL SEAfp17N20 N- | SEAfp17 (SEQ. NO. 18) |
| | | terminal peptide of SEAfp17 SEQ. | |
| | | NO. 18 | |
| 214 | SEAfp17N20- | PEPTIDE (1)..(20) | Randomized N-terminal peptide |
| | backfront | MPIEEFNKAILTKTPQLQLA | of SEAfp17 (SEQ. NO. 18) |
| | | SEAfp17N20backfront randomized sequence | |
| | | of N-terminal 20 amino acids of | |
| | | SEAfp17 SEQ. NO. 18 | |
| 215 | SEAfp17N20- | PEPTIDE (1)..(20) | Randomized N-terminal peptide |
| | reverse | MPLIAKNFEEIALQLQPTKT | of SEAfp17 (SEQ. NO. 18) |
| | | SEAfp17N20reverse randomized sequence | |
| | | of N-terminal 20 amino acids of SEAfp17 | |
| | | SEQ. NO. 18 | |
| 216 | SEAfp17N201- | PEPTIDE (1)..(20) | Randomized N-terminal peptide |
| | p1backforth | MLPITAKKTNPFQELEQILA | of SEAfp17 (SEQ. NO. 18) |
| | | SEAfp17N201p1backforth randomized | |
| | | sequence of N-terminal 20 amino acids | |
| | | of SEAfp17 SEQ. NO. 18 | |
| 217 | SEAfp17N202- | PEPTIDE (1)..(20) | Randomized N-terminal peptide |
| | p2forthback | MILPTKAKTFNPQEELQILA | of SEAfp17 (SEQ. NO. 18) |
| | | SEAfp17N202p2forthback randomized | |
| | | sequence of N-terminal 20 amino acids | |
| | | of SEAfp17 SEQ. NO. 18 | |
| 218 | YRAfp17N20 | PEPTIDE (1)..(20) | Truncated N-terminal peptide of |
| | YR17N20 | MPYFNKSKKNEIRPEKSKEE, YRAfp17N20 | YRAfp17 (SEQ. NO. 77) |
| | | N-terminal 20 amino acids of SEQ. NO. | |
| | | 77, gene accession code DJ39_RS03245 | |
| 219 | SFTox20 | PEPTIDE (1)..(20) | Truncated N-terminal peptide of |
| | | MPYSRESKEKEIHAKETERD, SFTox20 N- | PRT of accession code |
| | | terminal 20 amino acids of PRT of | WP_021808094.1 from Serratia |
| | | gene with accession code | fonticola |
| | | WP_021808094.1, protein with high | |
| | | identity to SEAfp18 (SEQ. NO. 19) | |
| 220 | EPTox20 | PEPTIDE (1)..(20) | Truncated N-terminal peptide of |
| | | MPYFNELNEKETRSKETESG, EPTox20 N- | PRT of accession code |
| | | terminal 20 amino acids of PRT of | WP_137270131.1 from Erwinia |
| | | gene with accession code | persicina |
| | | WP_137270131.1, protein with high | |
| | | identity to SEAfp18 (SEQ. NO. 19) | |
| 221 | YPTox20 | PEPTIDE (1)..(20) | Truncated N-terminal peptide of |
| | | MLYSSESKEKKTHSKETERD, YPTox20 N- | PRT of accession code |
| | | terminal 20 amino acids of PRT of | WP_049612744.1 from Yersinia |
| | | gene with accession code | pekkanenii |
| | | WP_049612744.1, protein with high | |
| | | identity to SEAfp18 (SEQ. NO. 19) | |
| 222 | SUTox20 | PEPTIDE (1)..(20) | Truncated N-terminal peptide of |
| | | MPYFRESKEKDTHAKESKQD, SUTox20 N- | PRT of accession code |
| | | terminal 20 amino acids of PRT of | WP_198774613.1 from Serratia |
| | | gene with accession code | ureilytica |
| | | WP_198774613.1, protein with high | |
| | | identity to SEAfp18 (SEQ. NO. 19) | |
| 223 | SMTox20 | PEPTIDE (1)..(20) | Truncated N-terminal peptide of |
| | | MPYSRESKEKDTHAKGSKQD, SMTox20 N- | PRT of accession code |
| | | terminal 20 amino acids of PRT of | AUO01772.1 from Serratia |
| | | gene with accession code AUO01772.1, | marcescens |
| | | protein with high identity to SEAfp18 | |
| | | (SEQ. NO. 19) | |
| 224 | SETox20 | PEPTIDE (1)..(20) | Truncated N-terminal peptide of |
| | | MPYSSESKLKDTHLKEAESD, SETox20 N- | PRT of accession code |
| | | terminal 20 amino acids of PRT of gene | HAU3143021.1 from Salmonella |
| | | with accession code HAU3143021.1, | enterica |
| | | protein with | |
| | | high identity to SEAfp18 (SEQ. NO. 19) | |
| 225 | Photopexin | PEPTIDE (1)..(20) | Truncated N-terminal peptide of |
| | NT20 | MNISSYFFLNEENIRFNNQC, Photopexin | PRT protein accession |
| | | NT20 N-terminal 20 amino acids of PRT | AXG42294 of gene accession |
| | | of gene PluDJC_08725 | code PluDJC_08725 from |
| | | | Photorhabdus luminescens |
| | | | GenBank: CP024900.1 |
| 226 | Toxin CyaA | PEPTIDE (1)..(20) | Truncated N-terminal peptide of |
| | NT20 | MPRYSNSQRTPTQSTKNTRR, Toxin CyaA | PRT of protein accession |
| | | NT20 N-terminal 20 amino acids of PRT | AXG42350 of gene accession |
| | | of gene PluDJC_08830 | code PluDJC_08830 from |
| | | | Photorhabdus luminescens |
| | | | GenBank: CP024900.1 |
| 227 | Cysteine | PEPTIDE (1)..(20) | Truncated N-terminal peptide of |
| | protease | MEHEYSEKEKPQKCPIQLRD, Cysteine | PRT of protein accession |
| | NT20 | protease NT20 N-terminal 20 amino | AXG43021 of gene accession |
| | | acids of PRT of gene PluDJC_12690 | code PluDJC_12690 from |
| | | | Photorhabdus luminescens |
| | | | GenBank: CP024900.1 |
| 228 | Cytotoxic | PEPTIDE (1)..(20) | Truncated N-terminal peptide of |
| | NF 1 NT20 | MLKYANPQTVATQRTKNTAK, cytotoxic | PRT of gene accession code |
| | | NF 1 NT20 N-terminal 20 amino acids of | PAU_RS16555 from |
| | | PRT of gene PAU_RS16555 | Photorhabdus asymbiotica |
| | | | GenBank: NC_012962, or |
| | | | WP_015835451 |
| 229 | YopT toxin | PEPTIDE (1)..(20) | Truncated N-terminal peptide of |
| | NT20 | MEREYNKKEKQKKSAIKLDD, YopT toxin | PRT of gene accession code |
| | | NT20 N-terminal 20 amino acids of PRT | PAU_RS10125 from |
| | | of gene PAU_RS10125 | Photorhabdus asymbiotica |
| | | | GenBank: NC_012962, or |
| | | | WP_015834232 |
| 230 | VC2_08635N20 | PEPTIDE (1)..(20) | Truncated N-terminal peptide of |
| | | MLSTEKHNKDTKHPRNREKK | PRT of gene accession code |
| | | VC2_08635N20 hypothetical protein, NT20 | PluDJC_08635 from |
| | | N-terminal 20 amino acids of PRT of | Photorhabdus luminescens |
| | | gene PluDJC_08635 hypothetical | GenBank: CP024900.1 |
| 231 | VC3_08730N20 | PEPTIDE (1)..(20) | Truncated N-terminal peptide of |
| | | MPNSKYSEKVNHSANGAEKC, | PRT of gene accession code |
| | | VC3_08730N20 hypothetical protein, NT20 | PluDJC_08730 from |
| | | N-terminal 20 amino acids of PRT of | Photorhabdus luminescens |
| | | gene PluDJC_08730 hypothetical | GenBank: CP024900.1 |
| 232 | VC6_13210N20 | PEPTIDE (1)..(20) | Truncated N-terminal peptide of |
| | | MYDSKKKNSEPTTKKKFERS, | PRT of gene accession code |
| | | VC6_13210N20 hypothetical protein, NT20 | PluDJC_13210 from |
| | | N-terminal 20 amino acids of PRT of | Photorhabdus luminescens |
| | | gene PluDJC_13210 hypothetical | GenBank: CP024900.1 |
| 233 | PAVC4_1PAU_ | PEPTIDE (1)..(20) | Truncated N-terminal peptide of |
| | RS13645 | MPNKKYSENTHQGKKPLIKS, | PRT of gene accession code |
| | | PAVC4_1PAU_RS13645 hypothetical, N- | PAU_RS13645 from |
| | | terminal 20 amino acids of PRT of gene | Photorhabdus asymbiotica |
| | | PAU_RS13645 | GenBank: NC_012962 |
| 234 | PAVC4_2PAU_ | PEPTIDE (1)..(20) | Truncated N-terminal peptide of |
| | RS22355 | MEREYSEKEKHKKHPIQLRD, | PRT of gene accession code |
| | | PAVC4_2PAU_RS22355 hypothetical | PAU_RS22355 from |
| | | protein, N-terminal 20 amino acids of | Photorhabdus asymbiotica |
| | | PRT of gene PAU_RS22355 | GenBank: NC_012962 |
| 235 | PAVC4_3PAU_ | PEPTIDE (1)..(20) | Truncated N-terminal peptide of |
| | RS13655 | MVHEYSINDRQKRHSFSSAN, | PRT of gene accession code |
| | | PAVC4_3PAU_RS13655 hypothetical | PAU_RS13655 from |
| | | protein, N-terminal 20 amino acids of | Photorhabdus asymbiotica |
| | | PRT of gene PAU_RS13655 | GenBank: NC_012962 |
| 236 | PAU_RS10120 | PEPTIDE (1)..(20) | Truncated N-terminal peptide of |
| | RHS repeat | MISTFDPAICAGTPTVTVLD, PAU_RS10120 | PRT of gene accession code |
| | protein | RHS repeat protein, N-terminal 20 amino | PAU_RS10120 from |
| | | acids of PRT of gene PAU_RS10120 | Photorhabdus asymbiotica |
| | | | GenBank: NC_012962 |
| 237 | ETTox20 | PEPTIDE (1)..(20) | Truncated N-terminal peptide of |
| | | MPYCNASKQKETYLKEIERD, ETTox20 | PRT of gene accession code |
| | | hypothetical protein, N-terminal 20 | WP_012442690.1 with high |
| | | amino acids of PRT WP_012442690.1 from | homology to SEAfp18N20 SEQ. |
| | | Erwinia tasmaniensis | NO. 87, 104 |
| 238 | PAVC4_4PAU_ | PEPTIDE (1)..(20) | Truncated N-terminal peptide of |
| | RS13660 | MNISSYFFLNEENIKENNQY, | PRT of gene accession code |
| | photopexin | PAVC4_4PAU_RS13660 photopexin, N- | PAU_RS13660 from |
| | | terminal 20 amino acids of PRT of gene | Photorhabdus asymbiotica |
| | | PAU_RS13660 | GenBank: NC_012962 |
| 239 | VC1_08535N20 | PEPTIDE (1)..(20) | Truncated N-terminal peptide of |
| | | LLTHNLIFRVKCKYSICCLF, | PRT of gene accession code |
| | | VC1_08535N20, NT20 N-terminal 20 amino | PluDJC_08535 from |
| | | acids of PRT of gene PluDJC_08535 | Photorhabdus luminescens |
| | | | GenBank: CP024900.1 |
| 240 | VC1_08530N20 | PEPTIDE (1)..(20) | Truncated N-terminal peptide of |
| | | LCAFPIDGYTNERANQGCGE, | PRT of gene accession code |
| | | VC1_08530N20 hypothetical protein, NT20 | PluDJC_08530 from |
| | | N-terminal 20 amino acids of PRT of | Photorhabdus luminescens |
| | | gene PluDJC_08530 | GenBank: CP024900.1 |
| 241 | VC2_08630N20 | PEPTIDE (1)..(20) | Truncated N-terminal peptide of |
| | | IIFTRDRNPTLSAHIKGGKK, | PRT of gene accession code |
| | | VC2_08630N20 hypothetical protein, NT20 | PluDJC_08630 from |
| | | N-terminal 20 amino acids of PRT of | Photorhabdus luminescens |
| | | gene PluDJC_08630 | GenBank: CP024900.1 |
| 242 | VC2_08640N20 | PEPTIDE (1)..(20) | Truncated N-terminal peptide of |
| | | EVNNGGNKSKAQAHTPDLVM, | PRT of gene accession code |
| | | VC2_08640N20, NT20 N-terminal 20 amino | PluDJC_08640 from |
| | | acids of PRT of gene PluDJC_08640 | Photorhabdus luminescens |
| | | | GenBank: CP024900.1 |
| 243 | VC3_08735N20 | PEPTIDE (1)..(20) | Truncated N-terminal peptide of |
| | | ELKHDDSKIKSQVSIPNLVK, | PRT of gene accession code |
| | | VC3_08735N20, NT20 N-terminal 20 amino | PluDJC_08735 from |
| | | acids of PRT of gene PluDJC_08735 | Photorhabdus luminescens |
| | | | GenBank: CP024900.1 |
| 244 | VC5_12680N20 | PEPTIDE (1)..(20) | Truncated N-terminal peptide of |
| | riboflavin | TIVNPYSIYLKHIPNGFQDA, | PRT of gene accession code |
| | | VC5_12680N20 riboflavin, NT20 N- | PluDJC_12680 from |
| | | terminal 20 amino acids of PRT of gene | Photorhabdus luminescens |
| | | PluDJC_12680 | GenBank: CP024900.1 |
| 245 | VC6_13215N20 | PEPTIDE (1)..(20) | Truncated N-terminal peptide of |
| | | LGDDIMPISNLAKESEVRAV, | PRT of gene accession code |
| | | VC6_13215N20 hypothetical protein, NT20 | PluDJC_13215 from |
| | | N-terminal 20 amino acids of PRT of | Photorhabdus luminescens |
| | | gene PluDJC_13215 | GenBank: CP024900.1 |
| 246 | PAVC1_4_PAU_ | PEPTIDE (1)..(20) | Truncated N-terminal peptide of |
| | RS09730 DUF | VGNKNTPSRVKIFISALIFM, | PRT of gene accession code |
| | domain | PAVC1_4_PAU_RS09730 DUF domain | PAU_RS09730 from |
| | protein | protein, N-terminal 20 amino acids of | Photorhabdus asymbiotica |
| | | PRT of gene PAU_RS09730 | GenBank: NC_012962 |
| 247 | PAVC5_1PAU_ | PEPTIDE (1)..(20) | Truncated N-terminal peptide of |
| | RS16575 | MPRYANYQINPKQNIKNSHG, | PRT of gene accession code |
| | | PAVC5_1PAU_RS 16575 hypothetical | PAU_RS16575 from |
| | | protein, N-terminal 20 amino acids of | Photorhabdus asymbiotica |
| | | PRT of gene PAU_RS16575 | GenBank: NC_012962 |
| 248 | PAVC5_2PAU_ | PEPTIDE (1)..(20) | Truncated N-terminal peptide of |
| | RS16565 | MMFDENLECVNHEIINEKLD, | PRT of gene accession code |
| | | PAVC5_2PAU_RS16565 hypothetical | PAU_RS16565 from |
| | | protein, N-terminal 20 amino acids of | Photorhabdus asymbiotica |
| | | PRT of gene PAU_RS16565 | GenBank: NC_012962 |
| 249 | RP-1N20 | PEPTIDE (1)..(20) | Randomly generated peptide |
| | | SENKTKHNQQDSTAKDCWHE, RP-1N20 | using RandSeq Expasy tool with |
| | | randomized peptide 1, using RandSeq | high polar amino acid content |
| | | Expasy tool to generate peptides with | with SEQ. NO. 87, 104 |
| | | high polar amino acid content with SEQ. | SEAfp18N20 as model in % of |
| | | NO. 87, 104 SEAfp18N20 as model in % | polar amino acids |
| | | of polar amino acids | |
| 250 | RP-2N20 | PEPTIDE (1)..(20) | Randomly generated peptide |
| | | ESHSDDKHRDQETNQKTANK, RP-2N20 | using RandSeq Expasy tool with |
| | | randomized peptide 1, using RandSeq | high polar amino acid content |
| | | Expasy tool to generate peptides with | with SEQ. NO. 87, 104 |
| | | high polar amino acid content with | SEAfp18N20 as model in % of |
| | | SEQ. NO. 87, 104 SEAfp18N20 as model | polar amino acids |
| | | in % of polar amino acids | |
| 251 | RP-3N20 | PEPTIDE (1)..(20) | Randomly generated peptide |
| | | EDDSKDHFKQSRNQTEKHYN, RP-3N20 | using RandSeq Expasy tool with |
| | | randomized peptide 1, using RandSeq | high polar amino acid content |
| | | Expasy tool to generate peptides with | with SEQ. NO. 87, 104 |
| | | high polar amino acid content with | SEAfp18N20 as model in % of |
| | | SEQ. NO. 87, 104 SEAfp18N20 as model | polar amino acids |
| | | in % of polar amino acids | |
| 252 | RP-4N20 | PEPTIDE (1)..(20) | Randomly generated peptide |
| | | EDHVNKKKQHTTSSDQDINE, RP-4N20 | using RandSeq Expasy tool with |
| | | randomized peptide 1 | high polar amino acid content |
| | | | with SEQ. NO. 87, 104 |
| | | | SEAfp18N20 as model in % of |
| | | | polar amino acids |
| 253 | RP-5N20 | PEPTIDE (1)..(20) | Randomly generated peptide |
| | | MEHEYSEKEKPQGKKPLIKS, RP-5N20 | using RandSeq Expasy tool with |
| | | randomized peptide 1 | high polar amino acid content |
| | | | with SEQ. NO. 87, 104 |
| | | | SEAfp18N20 as model in % of |
| | | | polar amino |

The below Table 5 lists the DNA sequences disclosed herein by their SEQ. ID No., the name(s) applied in the description and Figures and sequence lists for the sequences, the key features of each sequence and further information about the sequence.

TABLE 5
Plasmid DNA sequence Table

| SEQ. | Name of sequence in | | |
|---|---|---|---|
| ID | description and | Key features corresponding to sequence | Information about the |
| NO. | figures | list | sequence |
| 1 | pBAD33_SE1-18, SE1- | primer_bind (1283) . . . (1305) primer | Serratia entomophila |
| | 18, 18, S. entomophila | pBAD33_lin_Nhel_RV | (SE) Afp Syringe with all |
| | (SE) Afp18, Afp18 | primer_bind (1286) . . . (1340) primer | 18 Open Reading |
| | | afp1_pBAD33_with_RBS_FW | Frames (ORF's) cloned |
| | | RBS (1306) . . . (1319) Ribosomal binding site | into pBAD33 expression |
| | | CDS (1320) . . . (1769) Afp1 | vector |
| | | CDS (1788) . . . (2852) Afp2 | |
| | | CDS (2872) . . . (4227) Afp3 | |
| | | CDS (4267) . . . (5517) Afp4 excluding STOP | |
| | | codon, overlap with Afp5 | |
| | | CDS (5520) . . . (5966) Afp5 without STOP | |
| | | overlapping with afp6 | |
| | | CDS (5969) . . . (6130) Afp6 without START and | |
| | | STOP overlapping with afp7 | |
| | | CDS (6133) . . . (6816) Afp7 without START and | |
| | | STOP codon overlapping with afp8 | |
| | | CDS (6819) . . . (8405) Afp8 without START MET | |
| | | overlapping with afp7 | |
| | | CDS (8426) . . . (8845) Afp9 without STOP | |
| | | overlap with afp10 | |
| | | CDS (8848) . . . (9267) Afp10 without START | |
| | | MET overlap with Afp9 | |
| | | CDS (9344) . . . (11164) Afp11 without STOP | |
| | | codon overlap with Afp12 | |
| | | CDS (11166) . . . (14051) Afp12 overlapping CDS | |
| | | with Afp11, start of coding sequence is missing | |
| | | START MET and SER | |
| | | primer_bind (13793) . . . (13829) primer | |
| | | afp_12500_RV_merge | |
| | | primer_bind (13800) . . . (13828) primer | |
| | | afp_12500_FW_merge | |
| | | CDS (14077) . . . (15987) Afp13 | |
| | | CDS (16044) . . . (17651) Afp14 | |
| | | CDS (17688) . . . (19778) Afp15 | |
| | | CDS (19885) . . . (20736) Afp16 | |
| | | CDS (20842) . . . (21918) Afp17 | |
| | | CDS (22120) . . . (29220) Afp18 | |
| | | primer_bind (29190) . . . (29240) primer | |
| | | afp18_pBAD33_RVlong | |
| | | primer_bind (29221) . . . (29242) primer | |
| | | pBAD33_lin_BamHI_FW | |
| 20 | pBAD33_SE1-18C4, | primer_bind (1283) . . . (1305) primer | Serratia entomophila |
| | SE1-18C4 | pBAD33_lin_Nhel_RV | (SE) Afp Syringe with all |
| | | primer_bind (1286) . . . (1340) primer | 17 Open Reading |
| | | afp1_pBAD33_with_RBS_FW | Frames (ORF's) and |
| | | CDS (1320) . . . (1769) Afp1 | Afp18 toxin truncated to |
| | | CDS (1788) . . . (2852) Afp2 | I1437 (Afp18-C4), cloned |
| | | CDS (2872) . . . (4227) Afp3 | into pBAD33 expression |
| | | CDS (4267) . . . (5517) Afp4 without STOP codon | vector |
| | | CDS (5520) . . . (5966) Afp5 without STOP codon | |
| | | CDS (5969) . . . (6130) Afp6 without START and STOP codon | |
| | | CDS (6133) . . . (6816) Afp7 without START and | |
| | | STOP codon | |
| | | CDS (6819) . . . (8405) Afp8 without START codon | |
| | | CDS (8426) . . . (8845) Afp9 without STOP codon | |
| | | CDS (8848) . . . (9267) Afp10 without START codon | |
| | | CDS (9344) . . . (11164) Afp11 without STOP codon | |
| | | primer_bind (10959) . . . (10980) primer | |
| | | SEAfp11_merge_RV | |
| | | primer_bind (10961) . . . (10982) primer | |
| | | SEAfp11_merge_FW | |
| | | CDS (11166) . . . (14051) Afp12 without START codons MS | |
| | | CDS (14077) . . . (15987) Afp13 | |
| | | CDS (16044) . . . (17651) Afp14 | |
| | | CDS (17688) . . . (19778) Afp15 | |
| | | CDS (19855) . . . (20736) Afp16 | |
| | | CDS (20842) . . . (21918) Afp17 | |
| | | CDS (22120) . . . (26433) Afp18C4 C-terminal | |
| | | truncation I1437 | |
| | | primer_bind (26400) . . . (26444) primer | |
| | | SEAfp18C4trunc_pBAD33OH_RV | |
| | | primer_bind (26419) . . . (26455) primer | |
| | | pBAD33OH_SEAfp18C4_FW | |
| | | primer_bind (26434) . . . (26455)primer | |
| | | pBAD33_lin_BamHI_FW | |
| 39 | pBAD33_SE1-17, SE1- | primer_bind (1283) . . . (1305) primer | Serratia entomophila |
| | 17 | pBAD33_lin_Nhel_RV | (SE) Afp Syringe with 17 |
| | | primer_bind (1286) . . . (1340) primer | Open Reading Frames |
| | | afp1_pBAD33_with_RBS_FW | (ORF's) modified lacking |
| | | CDS (1320) . . . (1769) Afp1 | Afp18 toxin, cloned into |
| | | CDS (1788) . . . (2852) Afp2 | pBAD33 expression |
| | | CDS (2872) . . . (4227) Afp3 | vector |
| | | CDS (4267) . . . (5517) Afp4 without STOP codon | |
| | | CDS (5520) . . . (5966) Afp5 without STOP codon | |
| | | CDS (5969) . . . (6130) Afp6 without START and STOP codon | |
| | | CDS (6133) . . . (6816) Afp7 without START and STOP codon | |
| | | CDS (6819) . . . (8405) Afp8 without START codon | |
| | | CDS (8426) . . . (8845) Afp9 without STOP codon | |
| | | CDS (8848) . . . (9267) Afp10 without START codon | |
| | | CDS (9344) . . . (11164) Afp11 without STOP codon | |
| | | CDS (11166) . . . (14051) Afp12 without START codons MS | |
| | | primer_bind (13793) . . . (13829) primer | |
| | | afp_12500_RV_merge | |
| | | primer_bind (13800) . . . (13828) primer | |
| | | afp_12500_FW_merge | |
| | | CDS (14077) . . . (15987) Afp13 | |
| | | CDS (16044) . . . (17651) Afp14 | |
| | | CDS (17688) . . . (19778) Afp15 | |
| | | CDS (19855) . . . (20736) Afp16 | |
| | | CDS (20842) . . . (21918) Afp17 | |
| | | primer_bind (21885) . . . (21938) primer Se_Afp1- | |
| | | 17_pBAD33_RV | |
| | | primer_bind (21919) . . . (21940) primer | |
| | | pBAD33_lin_BamHI_FW | |
| 57 | pBAD33_SE1-16, SE1- | CDS (1320) . . . (1769) Afp1 | Serratia entomophila |
| | 16 | CDS (1788) . . . (2852) Afp2 | (SE) Afp Syringe with 16 |
| | | CDS (2872) . . . (4227) Afp3 | Open Reading Frames |
| | | CDS (4267) . . . (5517) Afp4 without STOP codon | (ORF's) modified lacking |
| | | CDS (5520) . . . (5966) Afp5 without STOP codon | |
| | | CDS (5969) . . . (6130) Afp6 without START and STOP codon | |
| | | CDS (6133) . . . (6816) Afp7 without START and STOP codon | |
| | | CDS (6819) . . . (8405) Afp8 without START | |
| | | primer_bind (8382) . . . (8424) primer SEAfp8- | |
| | | 9_merge_F2_RV | |
| | | primer_bind (8406) . . . (8435) primer SEAfp8- | |
| | | 9_merge_F1_FW | |
| | | CDS (8426) . . . (8845) Afp9 without STOP codon | |
| | | CDS (8848) . . . (9267) Afp10 without START codon | |
| | | CDS (9344) . . . (11164) Afp11 without STOP codon | |
| | | CDS (11166) . . . (14051) Afp12 without START | Afp17 and Afp18 toxin, |
| | | amino acids MS | cloned into pBAD33 |
| | | CDS (14077) . . . (15987) Afp13 | expression vector |
| | | CDS (16044) . . . (17651) Afp14 | |
| | | CDS (17688) . . . (19778) Afp15 | |
| | | CDS (19855) . . . (20736) Afp16 | |
| | | primer_bind (20705) . . . (20755) primer | |
| | | SEAfp16_pBAD33OH_F1_RV | |
| | | primer_bind (20728) . . . (20761) primer | |
| | | pBAD33_SEAfp16OH_F2_FW | |
| 74 | pET11a_SEAfp18, | CDS (933) . . . (8033) SEAfp18 toxin | Serratia entomophila |
| | SEAfp18, Afp18, 18 | primer_bind (7993) . . . (8033) primer | toxin Afp18, subcloned |
| | | SE_Afp18_p11a_Stop_RV | into pET11a for |
| | | primer_bind (8031) . . . (8058) primer | overexpression |
| | | pET11a_C3CTS-Stop_lin_FW | |
| 76 | pET11a_YRAfp17, | CDS (5318) . . . (11689) Yersinia ruckeri Afp17, | Yersinia ruckeri toxin |
| | YRAfp17, YR17 | toxin | YRAfp17 (S. entomophila |
| | | primer_bind (11658) . . . (11709) primer | Afp18 homolog), |
| | | YR_Afp17_p11a_stop_RVpubl | subcloned into pET11a |
| | | primer_bind (11687) . . . (11714) primer | for overexpression |
| | | pET11a_C3CTS-Stop_lin_FW | |
| 78 | pET11a_{—} | primer_bind (876) . . . (932) primer | Photorhabdus |
| | Photopexin_PLUDJC_0 | NEWpET11aLinYRAfp17tox_RV | luminescens Photopexin |
| | 8520, Photopexin | primer_bind (913) . . . (971) primer | PluDJC_08520 |
| | | Photopexin_OH-for-pET11a_FW | subcloned into pET11a |
| | | CDS (933) . . . (1976) Photorhabdus luminescens | for overexpression |
| | | Photopexin PluDJC_08520 | |
| | | primer_bind (1949) . . . (1993) primer | |
| | | Photopexin_OH-for-pET11a_RV | |
| | | primer_bind (1974) . . . (2001) primer | |
| | | pET11a_C3CTS-Stop_lin_FW | |
| 80 | pET11a_SEAfp18- | CDS (933) . . . (1082) Afp18-NT50 | S. entomophila Afp18 N- |
| | NT50-LL37, | CDS (1083) . . . (1196) LL37 codon optimized | terminal 50 amino acids |
| | Afp18NT50-LL37, | | (NT50) fused to human |
| | 18NT50-LL37 | | antimicrobial peptide |
| | | | LL37 (codon optimized) |
| | | | subcloned into pET11a |
| | | | for overexpression |
| 83 | pET11a_SEAfp18- | CDS (933) . . . (1022) Afp18-NT30 | S. entomophila Afp18 N- |
| | NT30-LL37, | CDS (1023) . . . (1136) LL37 codon optimized | terminal 30 amino acids |
| | Afp18NT30-LL37, | | (NT30) fused to human |
| | 18NT30-LL37 | | antimicrobial peptide |
| | | | LL37 (codon optimized) |
| | | | subcloned into pET11a |
| | | | for overexpression |
| 86 | pET11a_Afp18NT20- | CDS (933) . . . (992) Afp18-NT20 | S. entomophila Afp18 N- |
| | LL37, Afp18NT20- | CDS (993) . . . (1106) LL37 codon optimized | terminal 20 amino acids |
| | LL37, 18NT20-LL37 | | (NT20) fused to human |
| | | | antimicrobial peptide |
| | | | LL37 (codon optimized) |
| | | | subcloned into pET11a |
| | | | for overexpression |
| 89 | pET11a_SEAfp18- | CDS (933) . . . (965) Afp18-NT11 | S. entomophila Afp18 N- |
| | NT11-LL37, | CDS (966) . . . (1079) LL37 codon optimized | terminal 11 amino acids |
| | Afp18NT11-LL37, | | (NT11) fused to human |
| | 18NT11-LL37 | | antimicrobial peptide |
| | | | LL37 (codon optimized) |
| | | | subcloned into pET11a |
| | | | for overexpression |
| 92 | pET11a_SEAfp18-NT5- | CDS (933) . . . (947) Afp18-NT5 | S. entomophila Afp18 N- |
| | LL37, Afp18NT5-LL37, | CDS (948) . . . (1061) LL37 codon optimized | terminal 5 amino acids |
| | 18NT5-LL37 | | (NT5) fused to human |
| | | | antimicrobial peptide |
| | | | LL37 (codon optimized) |
| | | | subcloned into pET11a |
| | | | for overexpression |
| 95 | pET11a_SEAfp18-NT2- | CDS (933) . . . (938) Afp18-NT2 | S. entomophila Afp18 N- |
| | LL37, Afp18NT2-LL37, | CDS (939) . . . (1052) LL37 codon optimized | terminal 2 amino acids |
| | 18NT2-LL37 | | (NT2) fused to human |
| | | | antimicrobial peptide |
| | | | LL37 (codon optimized) |
| | | | subcloned into pET11a |
| | | | for overexpression |
| 97 | pET11a_SEAfp18- | CDS (933) . . . (1082) Afp18-NT50 | S. entomophila Afp18 N- |
| | NT50-Casphi2, | CDS (1083) . . . (3356) Casphi2 codon optimized | terminal 50 amino acids |
| | Afp18NT50-Casphi2, | | (NT50) fused to |
| | 18NT50-Casphi2 | | Biggiephage Casphi2 |
| | | | (codon optimized) |
| | | | subcloned into pET11a |
| | | | for overexpression |
| 100 | pET11a_SEAfp18- | CDS (933) . . . (1022) Afp18-NT30 | S. entomophila Afp18 N- |
| | NT30-Casphi2, | CDS (1023) . . . (3296) Casphi2 codon optimized | terminal 30 amino acids |
| | Afp18NT30-Casphi2, | | (NT30) fused to |
| | 18NT30-Casphi2 | | Biggiephage Casphi2 |
| | | | (codon optimized) |
| | | | subcloned into pET11a |
| | | | for overexpression |
| 103 | pET11a_Afp18NT20- | CDS (933) . . . (992) Afp18-NT20 | S. entomophila Afp18 N- |
| | Casphi2, Afp18NT20- | CDS (993) . . . (3266) Casphi2 codon optimized | terminal 20 amino acids |
| | Casphi2, 18NT20- | | (NT20) fused to |
| | Casphi2 | | Biggiephage Casphi2 |
| | | | (codon optimized) |
| | | | subcloned into pET11a |
| | | | for overexpression |
| 106 | pET11a_SEAfp18- | CDS (933) . . . (965) Afp18-NT11 | S. entomophila Afp18 N- |
| | NT11-Casphi2, | CDS (966) . . . (3239) Casphi2 codon optimized | terminal 11 amino acids |
| | Afp18NT11-Casphi2, | | (NT11) fused to |
| | 18NT11-Casphi2 | | Biggiephage Casphi2 |
| | | | (codon optimized) |
| | | | subcloned into pET11a |
| | | | for overexpression |
| 109 | pET11a_SEAfp18-NT5- | CDS (933) . . . (947) Afp18-NT5 | S. entomophila Afp18 N- |
| | Casphi2, Afp18NT5- | CDS (948) . . . (3221) Casphi2 codon optimized | terminal 5 amino acids |
| | Casphi2, 18NT5- | | (NT5) fused to |
| | Casphi2 | | Biggiephage Casphi2 |
| | | | (codon optimized) |
| | | | subcloned into pET11a |
| | | | for overexpression |
| 112 | pET11a_SEAfp18-NT2- | CDS (933) . . . (938) Afp18-NT2 | S. entomophila Afp18 N- |
| | Casphi2, Afp18NT2- | CDS (939) . . . (3212) Casphi2 codon optimized | terminal 2 amino acids |
| | Casphi2, 18NT2- | | (NT2) fused to |
| | Casphi2 | | Biggiephage Casphi2 |
| | | | (codon optimized) |
| | | | subcloned into pET11a |
| | | | for overexpression |
| 114 | pET11a_SEAfp18-C1, | CDS (933) . . . (7478) Afp18 C terminal truncation | Afp18 C terminal |
| | SEAfp18-C1, C1 | at L2181 | truncation at L2181 |
| | | | cloned into pET11a |
| | | | expression vector |
| 116 | pET11a_SEAfp18-C2, | CDS (933) . . . (6524) Afp18 C-terminal truncation | Afp18 C-terminal |
| | SEAfp18-C2, C2 | at A1863 | truncation at A1863 |
| | | | cloned into pET11a |
| | | | expression vector |
| 118 | pET11a_SEAfp18-C3, | CDS (933) . . . (6017) Afp18 C-terminal truncation | Afp18 C-terminal |
| | SEAfp18-C3, C3 | E1694 | truncation E1694 cloned |
| | | | into pET11a expression |
| | | | vector |
| 120 | pET11a_SEAfp18-C4, | CDS (933) . . . (5246) Afp18 C-terminal truncation | Afp18 C-terminal |
| | SEAfp18-C4, C4 | I1437 | truncation I1437 cloned |
| | | | into pET11a expression |
| | | | vector |
| 122 | pET11a_SEAfp18-C6, | CDS (933) . . . (2513) Afp18 C-terminal | Afp18 C-terminal |
| | SEAfp18-C6, C6 | truncation K526 | truncation K526 cloned |
| | | | into pET11a expression |
| | | | vector |
| 124 | pET11a_SEAfp18-N7, | CDS (933) . . . (1490) Afp18 N-terminal truncation | Afp18 N-terminal |
| | SEAfp18-N7, N7 | (M) 2182 | truncation (M) 2182 |
| | | | cloned into pET11a |
| | | | expression vector |
| 126 | pET11a_SEAfp18-N6, | CDS (933) . . . (2444) Afp18 N-terminal truncation | Afp18 N-terminal |
| | SEAfp18-N6, N6 | (M) 1864 | truncation (M) 1864 |
| | | | cloned into pET11a |
| | | | expression vector |
| 128 | pET11a_SEAfp18-N5, | CDS (933) . . . (2951) Afp18 N-terminal truncation | Afp18 N-terminal |
| | SEAfp18-N5, N5 | (M) 1695 | truncation (M) 1695 |
| | | | cloned into pET11a |
| | | | expression vector |
| 130 | pET11a_SEAfp18-N4, | CDS (933) . . . (3722) Afp18 N-terminal truncation | Afp18 N-terminal |
| | SEAfp18-N4, N4 | (M) 1438 | truncation (M) 1438 |
| | | | cloned into pET11a |
| | | | expression vector |
| 132 | pET11a_SEAfp18-N2, | CDS (933) . . . (6455) Afp18 N-terminal truncation | Afp18 N-terminal |
| | SEAfp18-N2, N2 | (M) 527 | truncation (M) 527 cloned |
| | | | into pET11a expression |
| | | | vector |
| 134 | pET11a_SEAfp18-N1, | CDS (933) . . . (7373) Afp18 N-terminal truncation | Afp18 N-terminal |
| | SEAfp18-N1, N1 | M221 | truncation M221 cloned |
| | | | into pET11a expression |
| | | | vector |
| 136 | pET11a_SEAfp18-NX1, | CDS (933) . . . (7520) Afp18 N-terminal truncation | Afp18 N-terminal |
| | SEAfp18-NX1, NX1 | M172 | truncation M172 cloned |
| | | | into pET11a expression |
| | | | vector |
| 138 | pET11a_SEAfp18-NX2, | CDS (933) . . . (7751) Afp18 N-terminal | Afp18 N-terminal |
| | SEAfp18-NX2, NX2 | truncation A96 and insertion of a start | truncation A96 and |
| | | Methionine | insertion of a start |
| | | | Methionine cloned into |
| | | | pET11a expression |
| | | | vector |
| 140 | pET11a_SEAfp18-NX3, | CDS (933) . . . (7946) Afp18 N-terminal truncation | Afp18 N-terminal |
| | SEAfp18-NX3, NX3 | S30 and addition of start Methionine | truncation S30 and |
| | | | addition of start |
| | | | Methionine cloned into |
| | | | pET11a expression |
| | | | vector |
| 142 | pET11a_SEAfp18-C10, | CDS (933) . . . (1025) Afp18 C10 terminal | Afp18 C-terminal |
| | SEAfp18-C10, C10 | truncation S30 | truncation S30 cloned |
| | | | into pET11a expression |
| | | | vector |
| 144 | pET11a_SEAfp18-C9, | CDS (933) . . . (1220) SEAfp18 C terminal | Afp18 C terminal |
| | SEAfp18-C9, C9 | truncation A95 | truncation A95 cloned |
| | | | into pET11a expression |
| | | | vector |
| 146 | pET11a_SEAfp18-C8, | CDS (933) . . . (1448) Afp18 C8 truncation T171 | Afp18 C8 truncation T171 |
| | SEAfp18-C8, C8 | | cloned into pET11a |
| | | | expression vector |
| 148 | pET11a_SEAfp18-C7, | CDS (933) . . . (1595) Afp18 C7 terminal | Afp18 C7 terminal |
| | SEAfp18-C7, C7 | truncation R220 | truncation R220 cloned |
| | | | into pET11a expression |
| | | | vector |
| 150 | pET11a_ Afp18C8- | CDS (933) . . . (1445) Afp18-C8 C-terminal | Afp18 C8 truncation T171 |
| | Casphi2, Afp18C8- | truncation T171 | fused to Casphi2, cloned |
| | Casphi2, Afp18C8- | CDS (1446) . . . (3719) Casphi2 codon optimized | into pET11a expression |
| | Cashpi2, 18C8- | | vector |
| | Cashpi2 | | |
| 153 | pET11a_ Afp18C8- | CDS (933) . . . (1445) SEAfp18-C8 C-terminal | Afp18 C8 truncation T171 |
| | PAExoU, Afp18C8- | truncation T171 | fused to Pseudomonas |
| | PAExoU, 18C8- | CDS (1446) . . . (3509) Pseudomonas aeruginosa | aeruginosa Type III |
| | PAExoU | Type III Secrection System Effector ExoU | Secrection System |
| | | | Effector ExoU (PAExoU), |
| | | | cloned into pET11a |
| | | | expression vector |
| 156 | pET11a_ Afp18C8- | CDS (933) . . . (1445) SEAfp18-C8 C-terminal | Afp18 C8 truncation T171 |
| | PATse1, Afp18C8- | truncation T171 | fused to Pseudomonas |
| | PATse1, 18C8-PATse1 | CDS (1446) . . . (1910) Pseudomonas aeruginosa | aeruginosa Type VI |
| | | Type VI Secretion System Effector Tse1 | Secretion System |
| | | | Effector Tse1 (PATse1), |
| | | | cloned into pET11a |
| | | | expression vector |
| 159 | pET11a_Afp18C8- | CDS (933) . . . (1445) SEAfp18-C8 C-terminal | Afp18 C8 truncation T171 |
| | LL37, Afp18C8-LL37, | truncation T171 | fused to LL37 human |
| | 18C8-LL37 | CDS (1446) . . . (1559) LL37 human antimicrobial | antimicrobial peptide, |
| | | peptide, codon optimized for bacterial | codon optimized for |
| | | expression | bacterial expression, |
| | | | cloned into pET11a |
| | | | expression vector |
| 162 | pET11a_Afp18C6- | CDS (933) . . . (2510) SEAfp18-C6 C-terminal | Afp18 C6 truncation |
| | Cas9, Afp18C6-Cas9 | truncation K526 | K526 fused to Cas9 from |
| | | primer_bind (2489) . . . (2510) primer | Francisella novicida, |
| | | SEAfp18C6_RV | cloned into pET11a |
| | | primer_bind (2492) . . . (2546) primer | expression vector |
| | | Cas9_SE18C6OH_FW | |
| | | CDS (2511) . . . (6620) Cas9 from Francisella | |
| | | novicida | |
| | | primer_bind (6591) . . . (6637) primer | |
| | | Cas9_pET11aOH_RV | |
| | | primer_bind (6618) . . . (6645) primer | |
| | | pET11a_C3CTS-Stop_lin_FW | |
| 165 | pET11a_Afp18C10- | CDS (933) . . . (1022) SEAfp18-C10 C-terminal | Afp18-C10 C-terminal |
| | PAYopT- | truncation S30 | truncation S30 fused to |
| | RHS_PAU_RS10125, | primer_bind (999) . . . (1022) primer | Photorhabdus |
| | Afp18C10-PAYopT- | SEAfp18C10_RV | asymbiotica YopT |
| | RHS, C10-YopT-RHS | primer_bind (1003) . . . (1063) primer | (PAU_RS10125) and |
| | | PAlopT_Yoptox_SE18C10OH_FW | Photorhabdus |
| | | CDS (1023) . . . (1997) Photorhabdus asymbiotica | asymbiotica RHS repeat |
| | | YopT (PAU_RS10125) | containing protein |
| | | CDS (2097) . . . (4877) Photorhabdus asymbiotica | (PAU_RS10120), cloned |
| | | RHS repeat containing protein | into pET11a expression |
| | | (PAU_RS10120) | vector |
| | | primer_bind (4856) . . . (4894) primer PAlopT- | |
| | | RHStox_OHpET11a_RV | |
| | | primer_bind (4875) . . . (4902) primer | |
| | | pET11a_C3CTS-Stop_lin_FW | |
| 169 | pET11a_Afp18C8- | CDS (933) . . . (1445) SEAfp18-C8 C-terminal | Afp18-C8 C-terminal |
| | PLRtxA toxin | truncation T171 | truncation T171 fused to |
| | PluDJC_12685, | primer_bind (1425) . . . (1445) primer | Photorhabdus |
| | Afp18C8-PLRtxA, C8- | SEAfp18C8_RV | luminescens RtxA toxin |
| | RtxA | primer_bind (1426) . . . (1477) primer | (PluDJC_12685), cloned |
| | | PLDJCVC5_RtxA_OHSE18C8_FW | into pET11a expression |
| | | CDS (1446) . . . (3065) Photorhabdus | vector |
| | | luminescens RtxA toxin (PluDJC_12685) | |
| | | primer_bind (3033) . . . (3082) primer | |
| | | PLDJCVC5_RtxA_pET11aOH_RV | |
| | | primer_bind (3063) . . . (3090) primer | |
| | | pET11a_C3CTS-Stop_lin_FW | |
| 172 | pET11a_ Afp17-Afp18, | primer_bind (876) . . . (932) primer | Afp17 and Afp18 toxin |
| | Afp17-Afp18, SE17-18, | NEWpET11aLinYRAfp17tox_RV | cloned into pET11a |
| | 17-18 | primer_bind (912) . . . (959) primer | expression vector with |
| | | SEAfp17_OHpET11a_FW | native promoter for Afp18 |
| | | CDS (933) . . . (2009) SEAfp17 remnant toxin | |
| | | CDS (2211) . . . (9311) SEAfp18 toxin | |
| | | primer_bind (9271) . . . (9328) primer | |
| | | SEAfp18_stop_pET11a_RV | |
| | | primer_bind (9309) . . . (9336) primer | |
| | | pET11a_C3CTS-Stop_lin_FW | |
| 175 | pET11a_ Afp18-sfGFP, | CDS (933) . . . (8030) SEAfp18 | Afp18 toxin fused to |
| | Afp18-sfGFP | primer_bind (7993) . . . (8030) primer | superfolder GFP at the |
| | | afp18_nostop_RV | C-terminus cloned into |
| | | primer_bind (8011) . . . (8054) primer | pET11a vector for |
| | | sfGFP_SEAfp18OH_FW | overexpression |
| | | CDS (8031) . . . (8747) superfolder GFP (sfGFP) | |
| | | primer_bind (8719) . . . (8764) primer | |
| | | sfGFP_SEAfp18OH_RV | |
| | | primer_bind (8745) . . . (8772) primer | |
| | | pET11a_C3CTS-Stop_lin_FW | |
| 178 | pET11a_ Afp18-C- | primer_bind (5274) . . . (5317) primer | Afp18 toxin C-termial |
| | terminal-Twin Strep- | p11a_01_RV | Twin Strep Tag cloned |
| | Tag (C3CTS), Afp18-C- | primer_bind (5298) . . . (5342) primer | into pET11a for |
| | terminal-Twin Strep- | Afp18_pET11a_C-3CTS_FW | overexpression |
| | Tag (C3CTS), | CDS (5318) . . . (12415) SEAfp18 | |
| | Afp18C3CTS, | primer_bind (12378) . . . (12435) primer | |
| | 18C3CTS | Afp18_pET11a_C-3CTS_RV | |
| | | CDS (12416) . . . (12550) C-terminal Twin Strep | |
| | | Tag | |
| | | primer_bind (12416) . . . (12433) primer | |
| | | pET11a_C3CTS_lin_FW | |
| 181 | pET11a_Afp18C6- | CDS (933) . . . (2510) SEAfp18-C6 C-terminal | S. entomophila Afp18 C- |
| | sfGFP, Afp18C6- | truncation K526 | terminal truncation K526 |
| | sfGFP, 18C6-sfGFP | primer_bind (2489) . . . (2510) primer | (SEAfp18C6) fused to |
| | | SEAfp18C6_RV | sfGFP, subcloned into |
| | | primer_bind (2491) . . . (2534) primer | pET11a for |
| | | sfGFP_OHAfp18C6_FW | overexpression |
| | | CDS (2511) . . . (3224) superfolder GFP (sfGFP) | |
| | | CDS (3225) . . . (3818) Mxe GyrA intein | |
| | | CDS (3849) . . . (4007) C-terminal binding domain | |
| | | (CBD) | |
| | | primer_bind (3984) . . . (4024) primer | |
| | | sfGFP_OHAfp18C6_RV | |
| | | primer_bind (4005) . . . (4032) primer | |
| | | pET11a_C3CTS-Stop_lin_FW | |
| 186 | pET11a_Afp18C4- | CDS (933) . . . (5243) SEAfp18-C4 C-terminal | S. entomophila Afp18 C- |
| | sfGFP, Afp18C4- | truncation I1437 | terminal truncation I1437 |
| | sfGFP, 18C4-sfGFP | primer_bind (5213) . . . (5243) primer | (SEAfp18C4) fused to |
| | | SEAfp18C4_RV | sfGFP, subcloned into |
| | | primer_bind (5222) . . . (5267) primer | pET11a for |
| | | sfGFP_OHAfp18C4_FW | overexpression |
| | | CDS (5244) . . . (5957) superfolder GFP (sfGFP) | |
| | | CDS (5958) . . . (6551) Mxe GyrA intein | |
| | | CDS (6582) . . . (6740) C-terminal binding domain | |
| | | (CBD) | |
| | | primer_bind (6714) . . . (6757) primer | |
| | | sfGFP_OHAfp18C4_RV | |
| | | primer_bind (6738) . . . (6765) primer | |
| | | pET11a_C3CTS-Stop_lin_FW | |
| 191 | pET11a_Afp18C4- | CDS (933) . . . (5243) SEAfp18-C4 C-terminal | S. entomophila Afp18 C- |
| | Photopexin | truncation I1437 | terminal truncation I1437 |
| | PluDJC_08520, | primer_bind (5213) . . . (5243) primer | (SEAfp18C4) fused to |
| | Afp18C4-Photopexin, | SEAfp18C4_RV | Photorhabdus |
| | 18C4-PL | primer_bind (5223) . . . (5282) primer | luminescens Photopexin |
| | | PLDJCVC1_08520_OHSEAfp18C4_FW | PluDJC_08520, |
| | | CDS (5244) . . . (6287) Photorhabdus | subcloned into pET11a |
| | | luminescens Photopexin PluDJC_08520 | for overexpression |
| | | primer_bind (6260) . . . (6304) primer | |
| | | PLDJCVC1_08520_OHpET11a_RV | |
| | | primer_bind (6285) . . . (6312) primer | |
| | | pET11a_C3CTS-Stop_lin_FW | |
| 194 | pET11a_Afp18C6- | CDS (933) . . . (2510) SEAfp18-C6 C-terminal | S. entomophila Afp18 C- |
| | YRAfp17, Afp18C6- | truncation K526 | terminal truncation K526 |
| | YRAfp17, 18C6-YR17, | primer_bind (2489) . . . (2510) primer | (SEAfp18C6) fused to |
| | 18C6-YR | SEAfp18C6_RV | Yersinia ruckeri Afp17 |
| | | primer_bind (2491) . . . (2542) primer | (502-2123 amino acids, |
| | | YRAfp17_aa502_OHSEAfp18C6_FW | C6-YR), subcloned into |
| | | CDS (2511) . . . (7379) Yersinia ruckeri Afp17 | pET11a for |
| | | (502-2123 amino acids, C6-YR) | overexpression |
| | | primer_bind (7348) . . . (7399) primer | |
| | | YR_Afp17_p11a_stop_RVpubl | |
| | | primer_bind (7377) . . . (7404) primer | |
| | | pET11a_C3CTS-Stop_lin_FW | |
| 197 | pET11a_Afp18C4- | CDS (933) . . . (5243) SEAfp18-C4 C-terminal | S. entomophila Afp18 C- |
| | YRAfp17, Afp18C4- | truncation I1437 | terminal truncation I1437 |
| | YRAfp17, 18C4-YR17, | primer_bind (5213) . . . (5243) primer | (SEAfp18C4) fused to |
| | 18C4-YR | SEAfp18C4_RV | Yersinia ruckeri Afp17 |
| | | primer_bind (5223) . . . (5272) primer | (1437-2123, amino acids, |
| | | YRAfp17_aa1437_OHSEAfp18C4_FW | C4-YR), subcloned into |
| | | CDS (5244) . . . (7307) Yersinia ruckeri Afp17 | pET11a for |
| | | (1437-2123, amino acids, C4-YR) | overexpression |
| | | primer_bind (7276) . . . (7327) primer | |
| | | YR_Afp17_p11a_stop_RVpubl | |
| | | primer_bind (7305) . . . (7332) primer | |
| | | pET11a_C3CTS-Stop_lin_FW | |

### Methods Concerning Examples 6-9

### Casphi2 Fusion Constructs and Antibodies of SEAfp18NT20 Mutants and NtSPs of Different Species

Constructs for investigating 20 amino acid signal domains from other species, pet11a_YRAfp17NT20-Casphi2 (SEQ. NO. 254, 255, 256), pet11a_SETox20-Casphi2 (SEQ. NO. 257, 258, 259), pet11a_YPTox20-Casphi2 (SEQ. NO. 260, 261, 262), pet11a_CyaANT20-Casphi2 (SEQ. NO. 263, 264, 265), pet11a_EP-Tox20-Casphi2 (SEQ. NO. 266, 267, 268), pet11a_SEAfp17NT20-Casphi2 (SEQ. NO. 269, 270, 271), pet11a_ExoUNT20-Casphi2 (SEQ. NO. 272, 273, 274), and mutant variants of pet11a_SEAfp18N20KtA-Casphi2 (SEAfp18N20KtA, lysines to alanines, SEQ. NO. 275, 276, 277), pet11a_SEAfp18N20KTtA-Casphi2 (SEAfp18N20KTtA, lysines, threonines to alanines, SEQ. NO. 278, 279, 280), pet11a_SEAfp18N20EtA-Casphi2 (SEAfp18N20EtA, glutamic acids to alanines, SEQ. NO. 281, 282, 283) fused to Casphi2 were designed by the inventor and ordered for synthesis and sub-cloning by Genscript. Polyclonal rabbit antibodys binding to signal domains were selected and produced for detection purposes (produced by Genscript, SEQ. NO. 284-288).

### Mass Spectrometry Analysis of Purified Multiprotein Assembly Samples

Samples after purification using the method described under ‘Co-Expression and Purification of Afp syringes and Cargo’ were analyzed for protein species content. 100 μL of room temp 50 mM ammonium bicarbonate per sample, and added ~7.5 μg of purified proteins to this. Following this, 250 ng of sequencing-grade trypsin was added and incubated the samples overnight with mild agitation. Samples were reduced and alkylated (using TCEP and chloroacetamide at 10 mM) for a few minutes prior to peptide clean-up via high-pH StageTip procedure. Around 1 μg of digested protein was analyzed (~250 ng of salvaged peptide) per injection for each sample. All samples were analyzed as a series of test runs to approximate their concentrations (the reported concentrations in the provided samples were ~200-fold different between highest and lowest), with two technical replicate main runs. Quantitative standard deviations were <5% for most observations.

### Electron Microscopy (EM)

The Afp particle quality and integrity were investigated using negative-stain electron microscopy. For negative staining, aliquots of 4 μl of Afp samples were added onto copper grids coated with continuous carbon, washed with distilled water, and stained with 2% uranyl acetate. The grids were dried at room temperature and analyzed on a Morgagni 268 transmission electron microscope at 100 kV. Afp and its variants were investigated for sample purity and assembly state. Cryo-grids were prepared with purified Afp particles with an FEI Vitrobot Mark IV at 4° C. and 95% humidity. 4 μl of sample was applied with about 0.5 mg/mL concentration onto freshly glow-discharged S373-7-UAUF UltrAuFoil QF—R2/2 on 200 mesh Au with a 10 s waiting time and blotted using a blot force of −1, 10 seconds waiting time. Cryo-grid screening was performed on a Tecnai G2 20 TWIN 200 kV transmission electron microscope.

Movies were collected using the semi-automated acquisition program EPU (FE, Thermo Fisher Scientific) on a Titan Krios G2 microscope operated at 300 keV paired with a Falcon 3EC direct electron detector (FE, Thermo Fisher Scientific). Images were recorded in linear mode, at 75,000× magnification with a calibrated pixel size of 1.1 Å and under focus range of 0.5 to 2.0 μm (0.3 μm steps) with a dose rate of 67.24 e-/Å2/s, 35 e-/Å2 and total exposure time of 0.59 s, 23 fractions 6,500 exposures (SE1-16); 69.87 e-/Å2/s, 39 e-/Å2, 0.59 s exposure time, 23 fractions 16,504 exposures (SE1-17); 67.26 e-/Å2/s, 38 e-/Å2, 0.60 s exposure time, 23 fractions 5,445 exposures (SE1-18C4); 69.87 e-/Å2/s, 39 e-/Å2, 0.57 s exposure time, 23 fractions, 9,741 exposures (SE1-18). Datasets SE1-16+18C8-Casphi2 and SE1-16+18C8-ExoU were collected on same Titan Krios G2 microscope operated at 300 keV but paired with a Falcon 4i direct electron detector (FE, Thermo Fisher Scientific). Images were recorded in linear mode, at 75,000× magnification with a calibrated pixel size of 0.749 Å and under focus range of 0.5 to 2.0 μm (0.3 μm steps) with a dose rate of 39 e-/Å2 and 7,014 exposures (SE1-16+18C8-Casphi2); dose rate of 43 e-/Å2 and 11,638 exposures (SE1-16+18C8-ExoU).

### Cryo-EM Data Processing and Analysis

All cryo-EM data processing was performed in cryoSPARC (Punjani, A. et al. 2017, Punjani, A. et al. 2020). For all datasets, movies were motion-corrected using full-frame or patch motion corrected and CTF was estimated using patch CTF estimation or CTFFIND4 (Rohou, A. et al.). Micrographs were inspected for CTF fit, motion correction and ice contamination.

### Baseplate Reconstructions

Particles were initially picked using a blob picker (particle diameter 400-600 A), extracted with a box size of 800 pix and downsampled to 600 pix and classified using 2D classification. Good classes containing base plates were then used in template-based picking. After 2D classification, ab initio models were con-structed and used for homogeneous 3D refinement with C6 symmetry imposed. Final 3D refinements were carried out with re-extracted base plates of 800 pix size (no down sampling). Maps were investigated and visualized using Chime-raX (Pettersen E F et al.).

Afp Particle Efficacy on Galleria mellonella Larvae

E coli BL21 star cells carrying the pBAD33 constructs SE1-16, SE1-17, SE1-18 and pBAD33 empty (used as a control) were grown and induced as described in section ‘Example 2—Production of high quality Afp syringes’. SE1-16 and toxin-chimeras were produced as described in ‘Co-Expression and Purification of Afp syringes and Cargo’ Thereafter, the cells were collected via centrifugation, 5,000 rpm for 20 minutes and washed 3 times with PBS 1× buffer. Protein extraction was performed via sonication followed by centrifugation, 5,000 rpm for 20 minutes, and filtration using a 0.2 μm filter to clear cells debris. To ensure that the syringe and toxin components were produced and in about the same amounts present in the protein lysate, immunodetection against toxin and Afp particle sheath was performed. For testing Afp particle activity, 10 G. mellonella larvae were injected with 30 μl of filtered protein lysates expressing the respective Afp constructs. 30 μl of PBS 1× buffer were injected as a control group to ensure that the solution used for the nanoparticle extraction was harmless to the larvae. The injected larvae were kept at 30° C. and observed for 13 days. After 13 days, the larvae injected with the PBS control and pBAD33 empty vector developed to moths, while the other ones not, here the experiment was stopped. Phenotypic interpretation was carried out as follows: category ‘dead larvae’ that upon pinch stress are not responsive and present a dark color, ‘arrested larvae’ that upon pinch stress were slightly responsive, however didn't progress in their development to moths in comparison with the control groups; ‘alive larvae’ that upon pinch stress were responsive, and along the 13 days of experiment developed to moths.

TABLE 6
Plasmid sequences

| SEQ | | | | |
|---|---|---|---|---|
| ID | | Feature | | |
| No. | Seq Name | Gene Name | position | Description |
| 254 | pet11a_YRAfp17NT20-Casphi2 | | | Nucleotide |
| | | | | sequence |
| 255 | pet11a_YRAfp17NT20-Casphi2 | YRAfp17NT20 | 933-992 | YRAfp17 N- |
| | | | | terminal signal |
| | | | | domain |
| 256 | pet11a_YRAfp17NT20-Casphi2 | Casphi2 | 993-3263 | Casphi2 enzyme |
| 257 | pet11a_SETox20-Casphi2 | | | Nucleotide |
| | | | | sequence |
| 258 | pet11a_SETox20-Casphi2 | SETox20 | 933-992 | SETox20 |
| | | | | Salmonella |
| | | | | enterica, signal |
| | | | | domain |
| 259 | pet11a_SETox20-Casphi2 | Casphi2 | 993-3263 | Casphi2 enzyme |
| 260 | pet11a_YPTox20-Casphi2 | | | Nucleotide |
| | | | | sequence |
| 261 | pet11a_YPTox20-Casphi2 | YPTox20 | 933-992 | YPTox20 Yersinia |
| | | | | pekkanenii, signal |
| | | | | domain |
| 262 | pet11a_YPTox20-Casphi2 | Casphi2 | 993-3263 | Casphi2 enzyme |
| 263 | pet11a_CyaANT20-Casphi2 | | | Nucleotide |
| | | | | sequence |
| 264 | pet11a_CyaANT20-Casphi2 | CyaANT20 | 933-992 | PLCyaANT20, |
| | | | | Photorhabdus |
| | | | | luminescens |
| | | | | PluDJC_08830 N- |
| | | | | terminal signal |
| | | | | domain of CyaA |
| | | | | toxin |
| 265 | pet11a_CyaANT20-Casphi2 | Casphi2 | 993-3263 | Casphi2 enzyme |
| 266 | pet11a_EPTox20-Casphi2 | | | Nucleotide |
| | | | | sequence |
| 267 | pet11a_EPTox20-Casphi2 | EPTox20 | 933-992 | EPTox20 Erwinia |
| | | | | persicina, N- |
| | | | | terminal signal |
| | | | | domain |
| 268 | pet11a_EPTox20-Casphi2 | Casphi2 | 993-3263 | Casphi2 enzyme |
| 269 | pet11a_SEAfp17NT20-Casphi2 | | | Nucleotide |
| | | | | sequence |
| 270 | pet11a_SEAfp17NT20-Casphi2 | SEAfp17NT20 | 933-992 | Serratia |
| | | | | entomophila Afp17 |
| | | | | N-terminal domain |
| 271 | pet11a_SEAfp17NT20-Casphi2 | Casphi2 | 993-3263 | Casphi2 enzyme |
| 272 | pet11a_ExoUNT20-Casphi2 | | | Nucleotide |
| | | | | sequence |
| 273 | pet11a_ExoUNT20-Casphi2 | ExoUNT20 | 933-992 | Pseudomonas |
| | | | | aeruginosa Type III |
| | | | | Secretion system |
| | | | | effector ExoU, N- |
| | | | | terminal signal |
| | | | | domain |
| 274 | pet11a_ExoUNT20-Casphi2 | Casphi2 | 993-3263 | Casphi2 enzyme |
| 275 | pet11a_SEAfp18N20KtA- | | | |
| | Casphi2 | | | |
| 276 | pet11a_SEAfp18N20KtA- | SEAfp18N20Kt | 933-992 | SEAfp18NT20 K to |
| | Casphi2 | A | | A mutations - |
| | | | | Serratia |
| | | | | entomophila |
| 277 | pet11a_SEAfp18N20KtA- | Casphi2 | 1053-3323 | Casphi2 enzyme |
| | Casphi2 | | | |
| 278 | pet11a_SEAfp18N20KTtA- | | | |
| | Casphi2 | | | |
| 279 | pet11a_SEAfp18N20KTtA- | SEAfp18N20K | 933-992 | |
| | Casphi2 | TtA | | |
| 280 | pet11a_SEAfp18N20KTtA- | Casphi2 | 1053-3323 | Casphi2 enzyme |
| | Casphi2 | | | |
| 281 | pet11a_SEAfp18N20EtA- | | | |
| | Casphi2 | | | |
| 282 | pet11a_SEAfp18N20EtA- | SEAfp18N20Et | 933-992 | |
| | Casphi2 | A | | |
| 283 | pet11a_SEAfp18N20EtA- | Casphi2 | 1053-3323 | Casphi2 enzyme |
| | Casphi2 | | | |

TABLE 7
Antibody (Protein) sequences

| Seq | | | |
|---|---|---|---|
| No | Seq Name | Antigen sequence | Description |
| 284 | EPTox20_ag | FNELNEKETRSKETC | Antigen to N-terminal signal |
| | | | domain EPTox20 Erwinia |
| | | | persicina, sequence: |
| | | | MPYFNELNEKETRSKETESG |
| 285 | CyaANT20_ag | YSNSQRTPTQSTKNC | Antigen to N-terminal signal |
| | | | domain PLCyaANT20, |
| | | | Photorhabdus luminescens |
| | | | PluDJC_08830 N-terminal |
| | | | signal domain of CyaA toxin |
| | | | sequence: |
| | | | MPRYSNSQRTPTQSTKNTRR |
| 286 | NT20ExoU_ag | ATASSLNQEPVETC | Antigen to N-terminal signal |
| | | | domain of Type III Secretion |
| | | | System Effector ExoU, |
| | | | sequence: |
| | | | MHIQSLGATASSLNQEPVET |
| 287 | SEAfp18NT20KtA_ag | SSESAEAETHSAETC | Antigen to N-terminal signal |
| | | | domain of SEAfp18NT20 |
| | | | lysine to alanine mutations, |
| | | | sequence: |
| | | | MPYSSESAEAETHSAETERD |
| 288 | SEAfp17NT20_ag | KTPQLQLAIEEFNKC | Antigen to N-terminal signal |
| | | | domain SEAfp17NT20, |
| | | | Serratia entomophila Afp17 |
| | | | remnant toxin, sequence: |
| | | | MPTKTPQLQLAIEEFNKAIL |

TABLE 8
N-terminal packing domains of CIS effectors and genomic syringe location

| | | | | Syringe Gene |
|---|---|---|---|---|
| Organism | peptide name | Accession Code | NT20 - peptide | Locations |
| S. entomophila | SEAfp18NT20 | AAT48355.1/ | MPYSSESKEKETHSKETERD | afp1(AAT48338)- |
| | (SEQ. NO. 87, | KHA73_24130 | | afp18 |
| | 104) | | | (AAT48355) |
| Y. ruckeri | YRAfp17N20 | DJ39_RS03245 | MPYFNKSKKNEIRPEKSKEE | DJ39_RS03165- |
| | (SEQ. NO. 218) | | | DJ39_RS03245 |
| Serratia fonticola | SFTox20 | WP_021808094.1 | MPYSRESKEKEIHAKETERD | L581_RS22600- |
| | (SEQ. NO. 219) | | | L581_RS22520 |
| Erwinia persicina | EPTox20 | WP_137270131.1 | MPYFNELNEKETRSKETESG | IFT93_RS22375- |
| | (SEQ. NO. 220) | | | IFT93_RS22455 |
| Yersinia pekkanenii | YPTox20 | WP_049612744.1 | MLYSSESKEKKTHSKETERD | AEP37_RS09525- |
| | (SEQ. NO. 221) | | | AEP37_RS09605 |
| Serratia ureilytica | SUTox20 | WP_198774613.1 | MPYFRESKEKDTHAKESKQD | JFQ86_16445- |
| | (SEQ. NO. 222) | | | JFQ86_RS16365 |
| Serratia marcescens | SMTox20 | AUO01772.1 | MPYSRESKEKDTHAKGSKQD | C0558_08345- |
| | (SEQ. NO. 223) | | | C0558_08275 |
| Salmonella enterica | SETox20 | HAU3143021.1 | MPYSSESKLKDTHLKEAESD | JDD69_004072- |
| | (SEQ. NO. 224) | | | JDD69_004088 |

TABLE 9
N-terminal packing domains of CIS effectors of different species and peptide properties

| | | | | Total polar | |
|---|---|---|---|---|---|
| | | | aa distribution | aa content | ACC(1,3) |
| Organism | gene name/peptide name | peptide | using protparam | (%) | (lag=2) |
| Y. ruckeri | YRAfp17N20 (SEQ. NO. 218) | MPYFNKSKKNEIRPEKSKEE | 5% (R),10% (N), 20% (E), | 75 | 1.516 |
| | | | 25% (K), 10% (S), 5% (Y) | | |
| S. entomophila | SEAfp18N20 (SEQ. NO. 87, 104) | MPYSSESKEKETHSKETERD | 5% (R), 5% (D), 25% (E), | 90 | −1.39 |
| | | | 5% (H), 15% (K), 20% (S), | | |
| | | | 10% (T), 5% (Y) | | |
| S. entomophila | SEAfp17N20 (SEQ. NO. 213) | MPTKTPQLQLAIEEFNKAIL | 5% (N), 10% (Q), 10% (E), | 45 | 1.981 |
| | | | 10% (K), 10% (T) | | |
| Serratia fonticola | SFTox20 (SEQ. NO. 219) | MPYSRESKEKEIHAKETERD | 10% (R), 25% (E),, 15% (K), | 70 | −1.163 |
| | | | 10% S, 5% (H), 5% (Y) | | |
| Erwinia persicina | EPTox20 (SEQ. NO. 220) | MPYFNELNEKETRSKETESG | 5% (R), 10% (N), 25% (E), | 70 | −2.109 |
| | | | 10% (K), 10% (S), 10% (T) | | |
| Yersinia pekkanenii | YPTox20 (SEQ. NO. 221) | MLYSSESKEKKTHSKETERD | 5% (R), 5% (D), 20% (E), | 80 | −2.15 |
| | | | 20% (K), 20% (S), 10% (T) | | |
| Serratia ureilytica | SUTox20 (SEQ. NO. 222) | MPYFRESKEKDTHAKESKQD | 5% (R),10% (D), 15% (E), | 75 | −0.904 |
| | | | 5% (H), 20% (K), 10% (S), | | |
| | | | 5% (T), 5% (Y) | | |
| Serratia marcescens | SMTox20 (SEQ. NO. 223) | MPYSRESKEKDTHAKGSKQD | 5% (R), 10% (D), 10% (E), | 75 | −0.789 |
| | | | 5% (H), 20% (K), 15% (S), | | |
| | | | 5% (T), 5% (Y) | | |
| Salmonella enterica | SETox20 (SEQ. NO. 224) | MPYSSESKLKDTHLKEAESD | 10% (D), 15% (E), 10% (L), | 80 | −1.76 |
| | | | 15% (K), 20% (S), 5% (T), | | |
| | | | 5% (Y) | | |
| P. luminescens | VC1_08520N20 photopexin (SEQ. NO. 225) | MNISSYFFLNEENIRFNNQC | 5% (R), 25% (N), 5%(Q), | 60 | −0.1579 |
| | | | 10% (E), 10% (S), 5% (Y) | | |
| P. luminescens | VC2_08635N20 hypothetical (SEQ. NO. 230) | MLSTEKHNKDTKHPRNREKK | 10% (R), 10%(N), 5% (D), | 85 | −2.164 |
| | | | 10%(E), 10%(H), 25%(K), | | |
| | | | 5%(S), 10%(T) | | |
| P. luminescens | VC3_08730N20 hypothetical (SEQ. NO. 231) | MPNSKYSEKVNHSANGAEKC | 15%(N), 10%(E), 5%(H), | 65 | −0.307 |
| | | | 15%(K), 15%(S), 5%(Y) | | |
| P. luminescens | VC4_08830N20 Toxin CyaA (SEQ. NO. 226) | MPRYSNSQRTPTQSTKNTRR | 20%(R), 10%(N), 10%(Q), | 85 | −1.273 |
| | | | 5%(K), 15%(S), 20%(T), 5% (Y) | | |
| P. luminescens | VC5_12690N20 Cysteine prot. (SEQ. NO. 227) | MEHEYSEKEKPQKCPIQLRD | 5%(R), 5%(D), 10%(Q), 20%(E), | 70 | −0.3316 |
| | | | 5%(H), 15%(K), 5%(S), 5%(Y) | | |
| P. luminescens | VC6_13210N20 hypothetical (SEQ. NO. 232) | MYDSKKKNSEPTTKKKFERS | 5%(R), 5%(N), 5%(D), 10%(E), | 85 | −1.2533 |
| | | | 30%(K), 15% (S), 10%(T), 5%(Y) | | |
| P. asymbiotica | PAVC4_1PAU_RS13645 hypothetical (SEQ. NO. | MPNKKYSENTHQGKKPLIKS | 10%(N), 5%(Q), 5%(E), 5%(H), | 70 | −0.247 |
| | 233) | | 25%(K), 10%(S), 5%(T), 5%(Y) | | |
| P. asymbiotica | PAVC4_2PAU_RS22355 hypothetical (SEQ. NO. | MEREYSEKEKHKKHPIQLRD | 10%(R), 5%(D), 5%(Q), 20%(E), | 80 | −1.2646 |
| | 234) | | 10%(H), 20%(K), 5%(S), 5%(Y) | | |
| P. asymbiotica | PAVC4_3PAU_RS13655 hypothetical (SEQ. NO. | MVHEYSINDRQKRHSFSSAN | 10%(R), 10%(N), 5%(D), 5%(Q), | 65 | −1.886 |
| | 235) | | 5%(E), 5%(K), 20%(S), 5%(Y) | | |
| P. asymbiotica | PAVC5_4PAU_RS16555 cytotoxicNF 1 (SEQ. NO. | MLKYANPQTVATQRTKNTAK | 5%(R), 10%(N), 10%(Q), 15%(K), | 65 | −1.136 |
| | 228) | | 20%(T), 5%(Y) | | |
| P. asymbiotica | PAU_RS10120 RHS repeat (SEQ. NO. 236) | MISTFDPAICAGTPTVTVLD | 10%(D), 5%(S), 20%(T), | 35 | −0.0745 |
| P. asymbiotica | PAU_RS10125 YopT (SEQ. NO. 229) | MEREYNKKEKQKKSAIKLDD | 5%(R), 5%(N), 10%(D), 5%(Q), | 80 | −1.242 |
| | | | 15%(E), 30%(K), 5%(S), 5%(Y) | | |
| Erwinia tasmaniensis | ETTox20 hypothetical protein (SEQ. NO. 237) | MPYCNASKQKETYLKEIERD | 5% (R), 5% (N), 5% (D), 5%(Q), | 65 | 0.186 |
| | | | 15% (E), 15%(K), 5%(S), 10%(Y) | | |
| P. asymbiotica | PAVC4_4PAU_RS13660 photopexin (SEQ. NO. 238) | MNISSYFFLNEENIKFNNQY | 25%(N), 5%(Q), 10%(E), 5%(K), | 65 | −0.843 |
| | | | 10%(S), 10%(Y) | | |
| P. luminescens | VC1_08535N20 pseudogene (SEQ. NO. 239) | LLTHNLIFRVKCKYSICCLF | 5%(R), 5%(N), 5%(H), 10%(K), | 40 | −1.143 |
| | | | 5%(S), 5%(T), 5%(Y) | | |
| P. luminescens | VC1_08530N20 hypothetical (SEQ. NO. 240) | LCAFPIDGYTNERANQGCGE | 5%(R), 10%(N), 5%(Q), 10%(E), | 40 | −0.068 |
| | | | 5%(T), 5%(Y) | | |
| P. luminescens | VC2_08630N20 hypothetical (SEQ. NO. 241) | IIFTRDRNPTLSAHIKGGKK | 10%(R), 5%(N), 5%(D), 5%(L), | 65 | −0.0125 |
| | | | 15%(K), 5%(S), 10%(T) | | |
| P. luminescens | VC2_08640N20 pseudogene (SEQ. NO. 242) | EVNNGGNKSKAQAHTPDLVM | 15%(N), 5%(D), 5%(Q), 5%(E), | 60 | −0.273 |
| | | | 5%(H), 10%(K), 5%(S), 5%(L), | | |
| | | | 5%(T) | | |
| P. luminescens | VC3_08735N20 pseudogene (SEQ. NO. 243) | ELKHDDSKIKSQVSIPNLVK | 5%(N), 10%(D), 5%(Q), 5%(E), | 70 | −0.87 |
| | | | 10%(L), 20%(K), 15%(S) | | |
| P. luminescens | VC5_12680N20 riboflavin (SEQ. NO. 244) | TIVNPYSIYLKHIPNGFQDA | 10%(N), 5%(D), 5%(Q), 5%(H), | 50 | −1.123 |
| | | | 5%(K), 5%(S), 5%(T), 10%(Y) | | |
| P. luminescens | VC6_13215N20 hypothetical protein (SEQ. | LGDDIMPISNLAKESEVRAV | 5%(R), 5%(N), 10%(D), 10%(E), | 55 | −1.432 |
| | NO. 245) | | 10%(L), 5%(K), 10%(S) | | |
| P. asymbiotica | PAVC1_4_PAU_RS09730 DUF domain protein | VGNKNTPSRVKIFISALIFM | 5%(R), 10%(N), 5%(L), 10%(K), | 45 | −0.38 |
| | (SEQ. NO. 246) | | 10%(S), 5%(T) | | |
| P. asymbiotica | PAVC5_1PAU_RS16575 hypothetical protein | MPRYANYQINPKQNIKNSHG | 5%(R), 20%(N), 10%(Q), 5%(H), | 65 | −1.072 |
| | (SEQ. NO. 247) | | 10%(K), 5%(S), 10%(Y) | | |
| P. asymbiotica | PAVC5_2PAU_RS16565 hypothetical protein | MMFDENLECVNHEIINEKLD | 15%(N), 10%(D), 20%(E), 5%(H) | 50 | −2.568 |
| | (SEQ. NO. 248) | | | | |
| S. entomophila | RP-1N20 randomized peptide 1, using | SENKTKHNQQDSTAKDCWHE | 10%(N), 15%(D), 10%(Q), 10%(E), | 90 | −0.542 |
| | RandSeq Expasy tool with SEQ. 87, 104 | | 10%(H), 15%(K), 10%(S), 10%(T) | | |
| | parameters as input (SEQ. NO. 249) | | | | |
| S. entomophila | RP-2N20 randomized peptide 1, using | ESHSDDKHRDQETNOKTANK | 5%(R), 10%(N), 15%(D), 10%(Q), | 85 | −0.779 |
| | RandSeq Expasy tool with SEQ. 87, 104 | | 10%(E), 10%(H), 15%K), 10%(S), | | |
| | parameters as input (SEQ. NO. 250) | | | | |
| S. entomophila | RP-3N20 randomized peptide 1, using | EDDSKDHFKQSRNQTEKHYN | 5%(R), 10%(N), 15%(D), 10%(Q), | 90 | −0.234 |
| | RandSeq Expasy tool with SEQ. 87, 104 | | 10%(E), 10%(H), 15%K), 10%(S), | | |
| | parameters as input (SEQ. NO. 251) | | 5% | | |
| S. entomophila | RP-4N20 randomized peptide 1, using | EDHVNKKKQHTTSSDQDINE | 10%(N), 15%(D), 10%(Q), 10%(E), | 80 | −0.1773 |
| | RandSeq Expasy tool witSEQ. 87, 104 | | 10%(H), 15%(K), 10%(S) | | |
| | parameters as input (SEQ. NO. 252) | | | | |

### Example 6—Functional Packing Domains from Different Species Loading Cargo into Afp

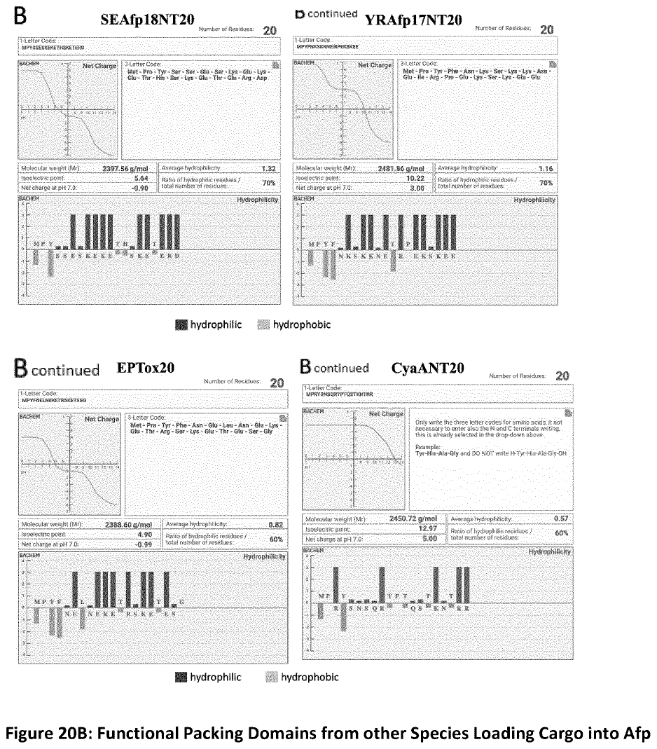

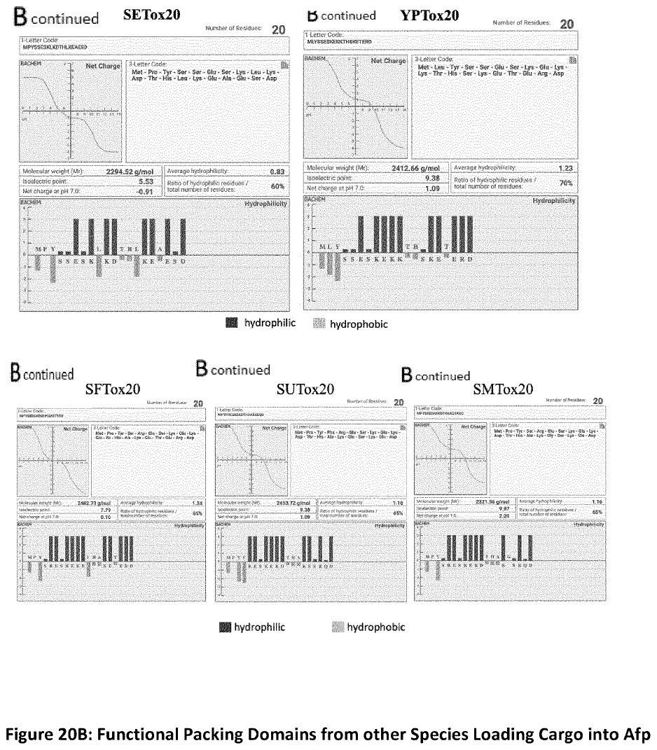

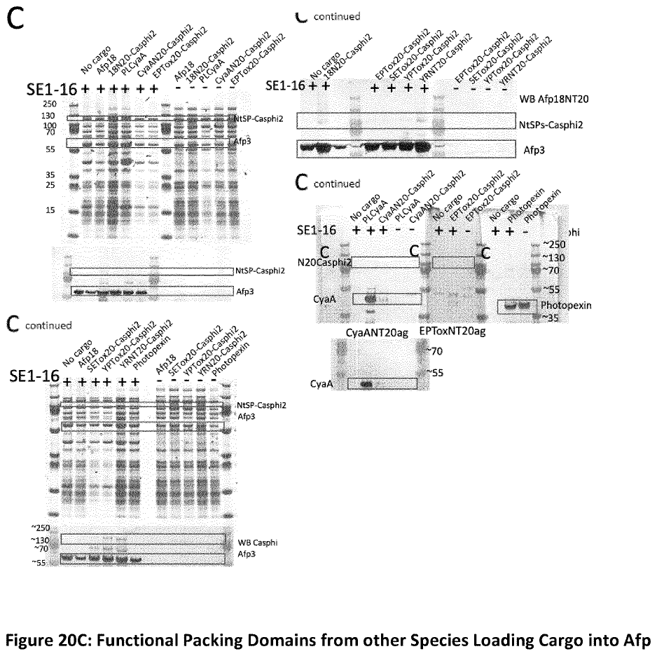

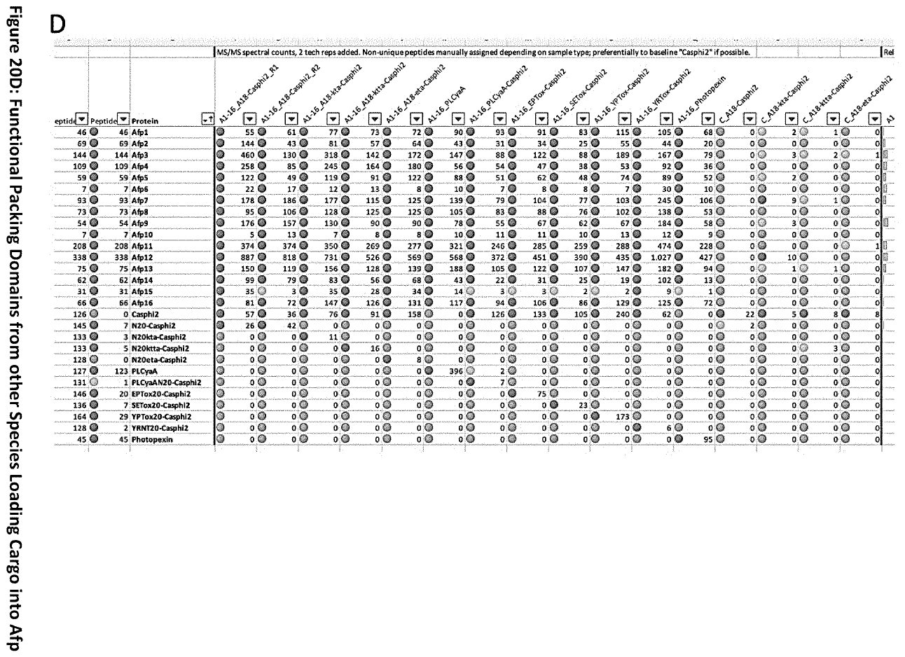

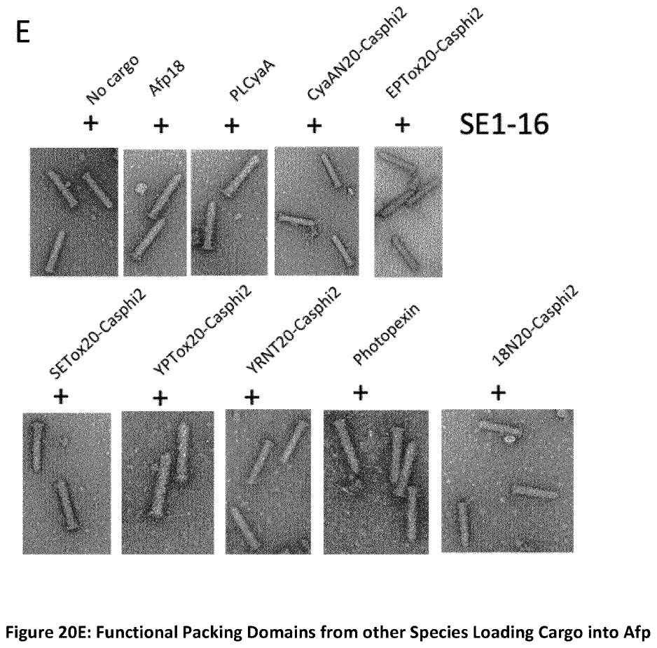

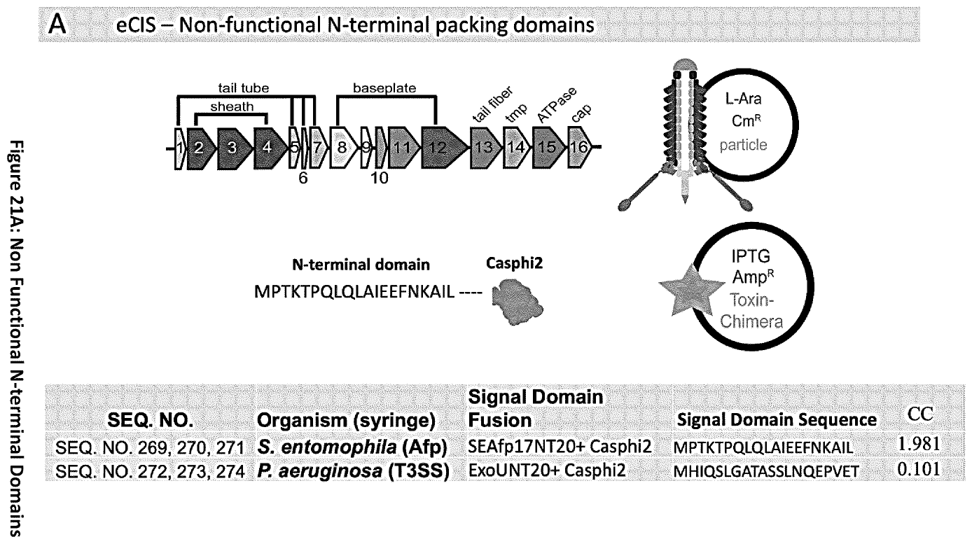

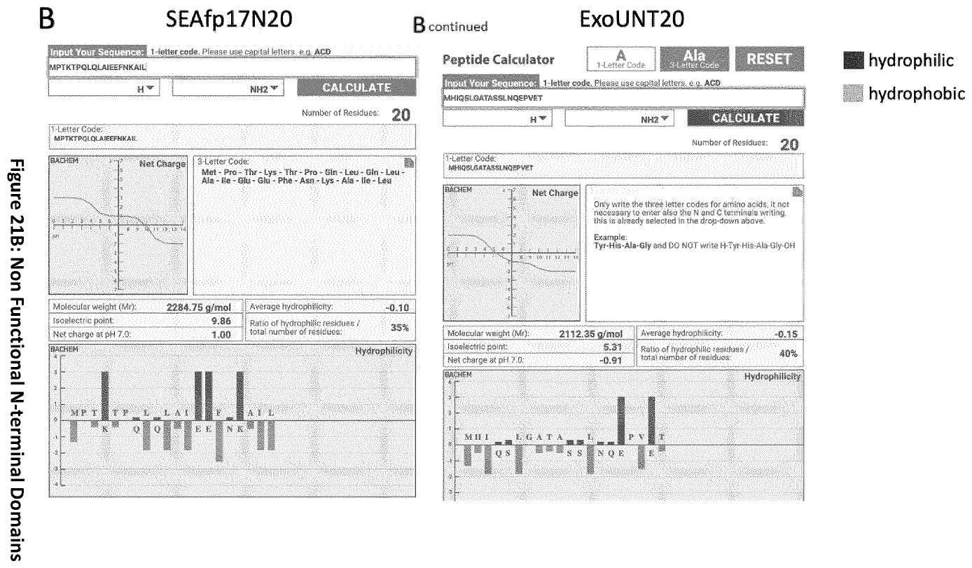

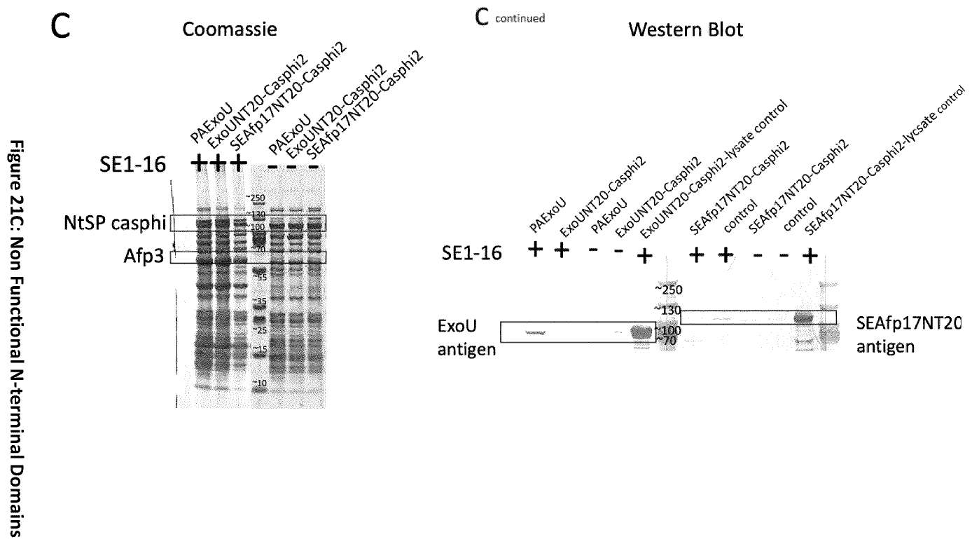

N-terminal packing domains from different CIS species effectors fused to non-CIS related Casphi2, and their packing capability into Afp was investigated. N-terminal signal domains (see FIGS. 18A and 19) with high negative CC values was chosen to investigate whether they can pack non syringe related cargo into Afp. Signal domains were chosen from a variety of bacterial species e.g., Salmonella enterica and Photorhabdus luminescens Virulence Cassettes (PVCs) (Table 8 & 9). Co-production of Afp syringe and signal domain chimeras was carried out. The results are shown in FIG. 20 (A)-(E).

Conclusion: N-terminal signal domains of CIS effectors from different species. (high negative CC value, high hydrophilicity content, absence of hydrophobic patches) can be used to load cargo into Afp. This highlights the broad application of those signal domains across CIS syringes.

### Example 7—Non Functional N-terminal Domains

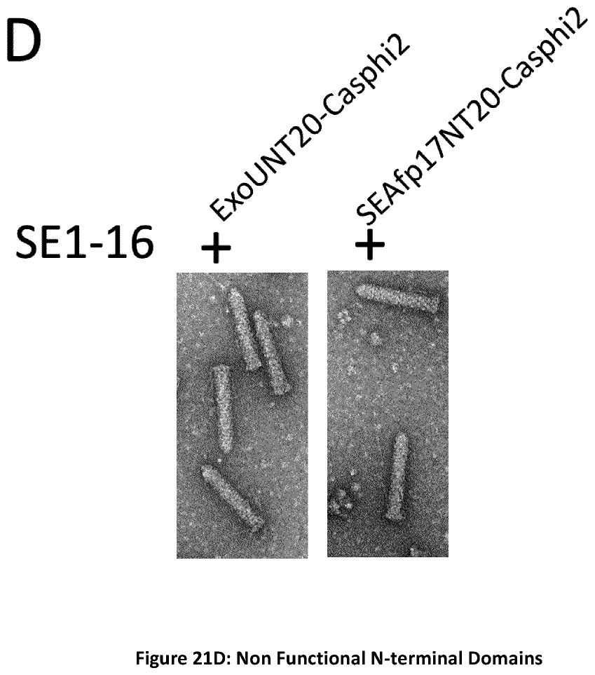

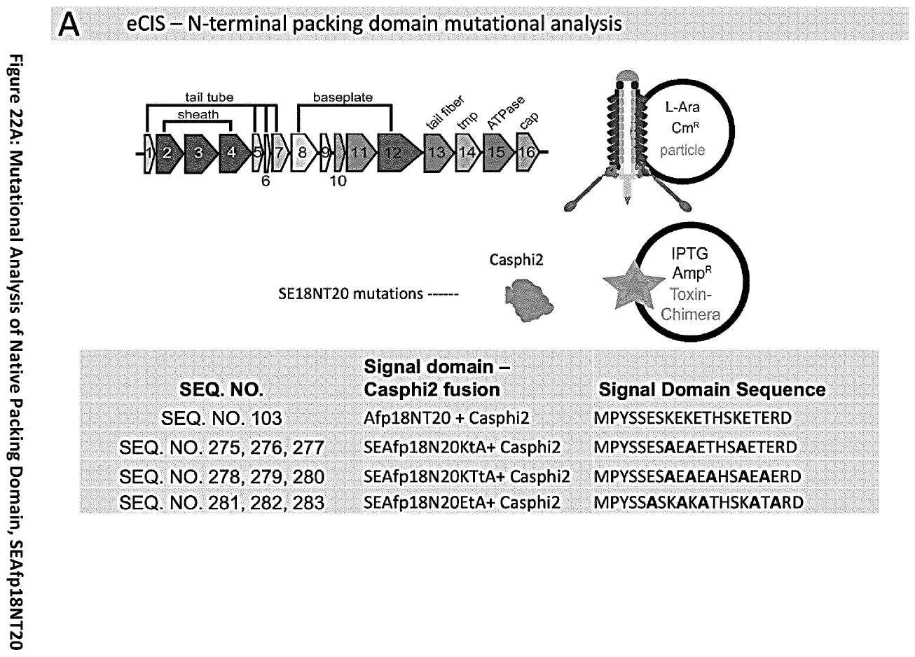

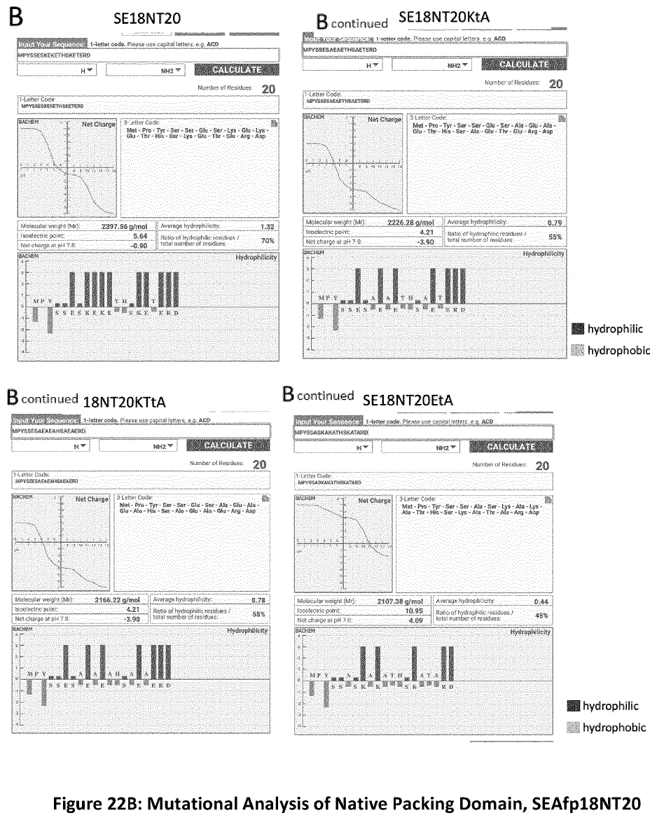

It was investigated whether potential non-qualified N-terminal signal domains selected based on physico-chemical properties (highly positive CC value, hydrophobicity) from Pseudomonas aeruginosa and Serratia entomophilla, could cause the packing domain to package Casphi2 into Afp. The results are shown in FIG. 21 (A)-(D).

Conclusion: 2 packing domains could be highlighted not packing Casphi2 into Afp, SEAfp17NT20 and ExoUNT20. Notably Afp18NT11 (SEQ. NO. 106) could as well not package Casphi2 into Afp (Table 3 & FIG. 12). Afp18NT11, a 11 amino acid version of functional packing domain Afp18NT20 fused to Casphi2, has low content of hydrophilic residues and more hydrophilic amino acids are required for successful packing.

### Example 8—Mutational Analysis of Native Packing Domain, SEAfp18NT20

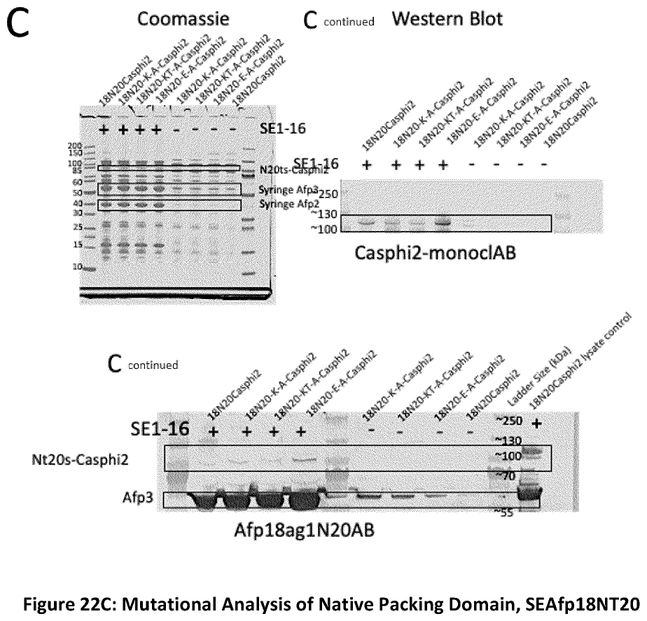

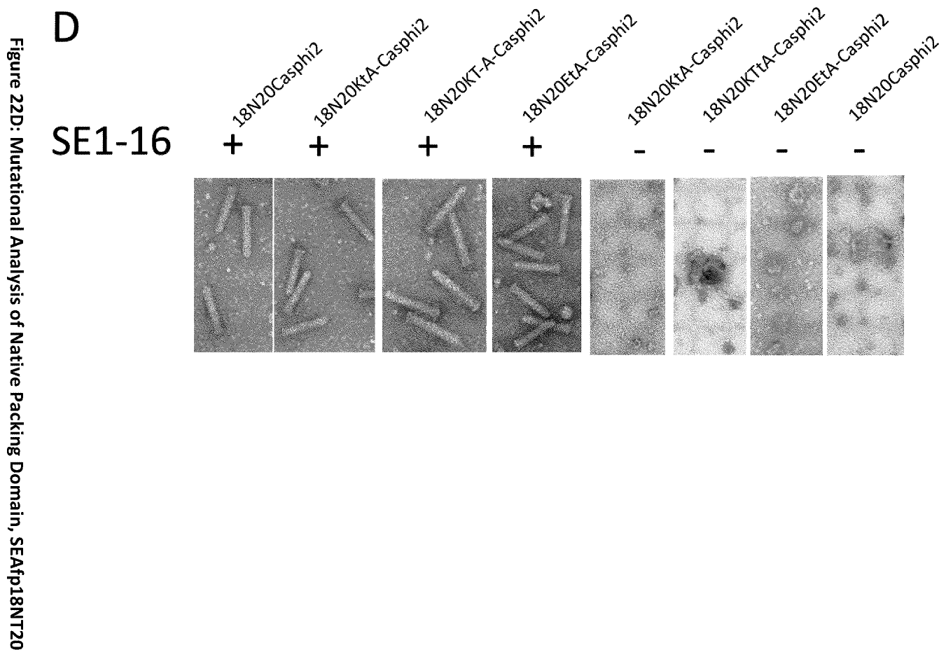

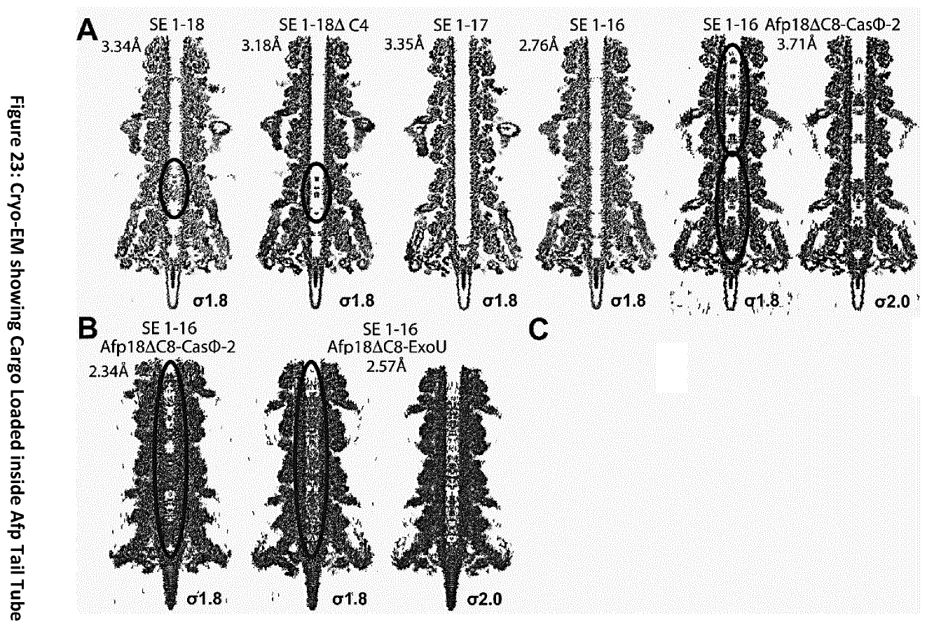

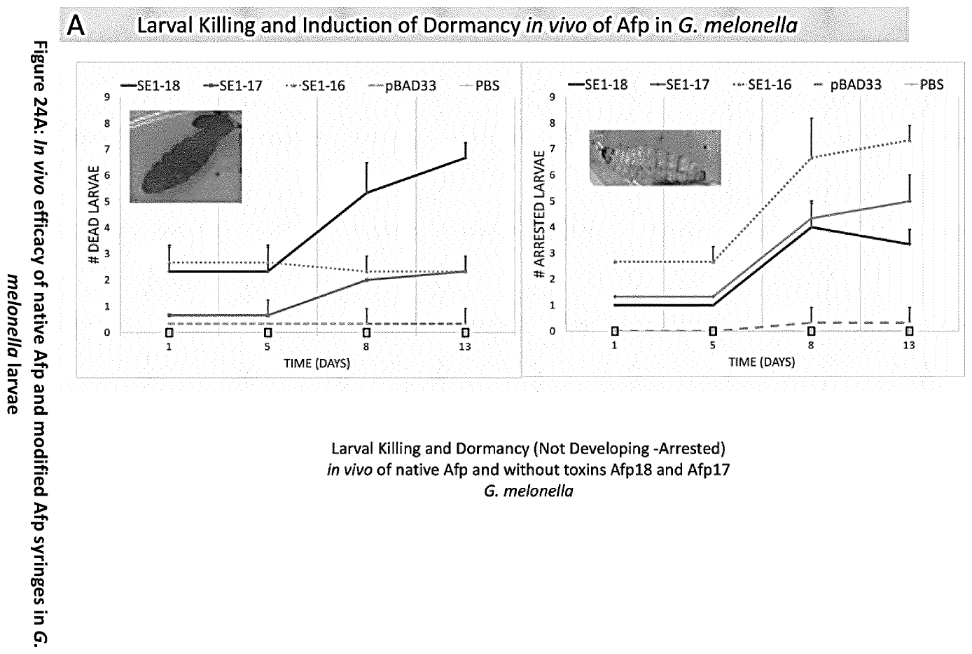

To further investigate the packing domains ablity to package Casphi2 into Afp Mutational analysis of native SEAfp18NT20 packing domain a mutational analysis of Afp's native packing domain SEAfp18NT20 (SEQ. NO. 103) was done. Hydrophilic residues were replaced by non-polar alanine. The results are shown in FIG. 22 (A)-(D).

Conclusion: The native SEAfp18NT20 domain can be mutated with up to 5 hydrophilic residues (K, T, E) along its 20 amino acid length and replaced by non-polar amino acids, showing functional packing. The results give insight into the packing domain range, detection of other packing domains and widens the range of design of new packing domains.

### Example 9: Cryo-EM Showing Cargo Loaded Inside Afp Tail Tube

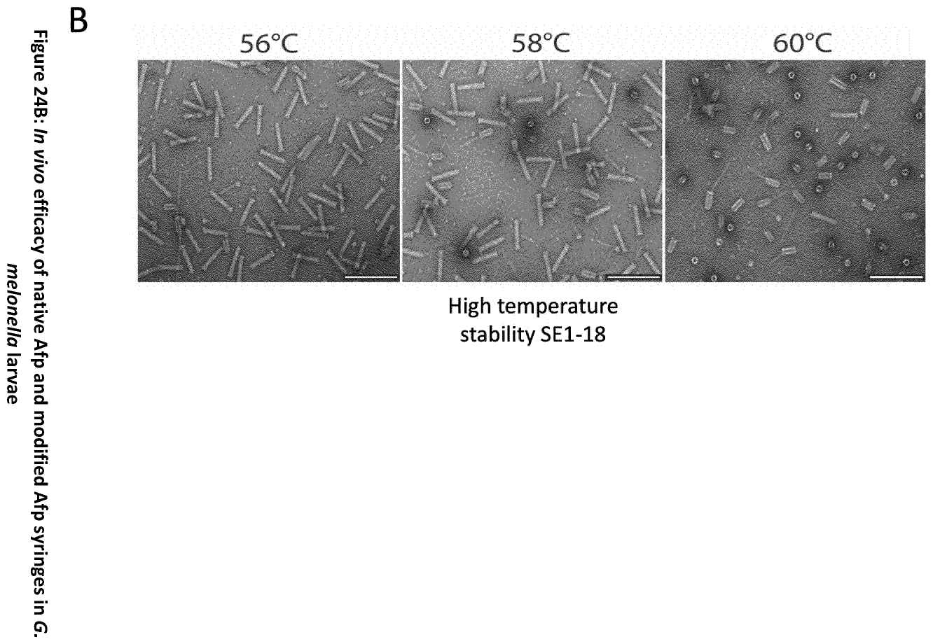

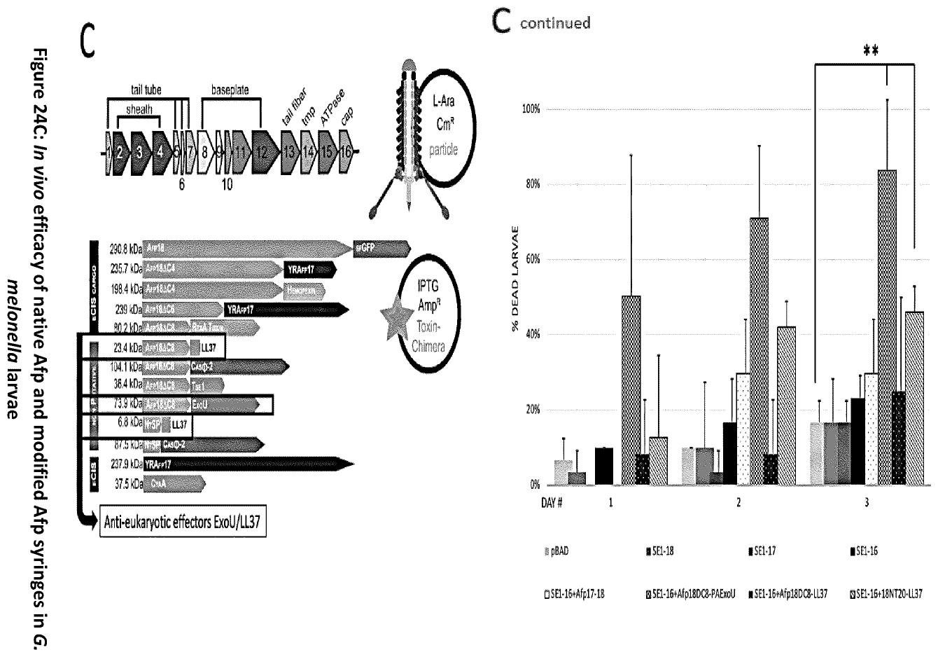

By Cryo-EM analysis two non-eCIS related effectors were investigated, if modified syringes could be loaded and if cargo effectors are located inside the tail tube. The results are shown in FIG. 23 (A)-(B).

Conclusion: Modified syringes show cargo loading inside the tail tube of two non-eCIS related effectors. This confirms modified cargos can be packed inside Afp.

### Example 10: In Vivo Efficacy of Native Afp and Modified Afp Syringes in G. melonella Larvae

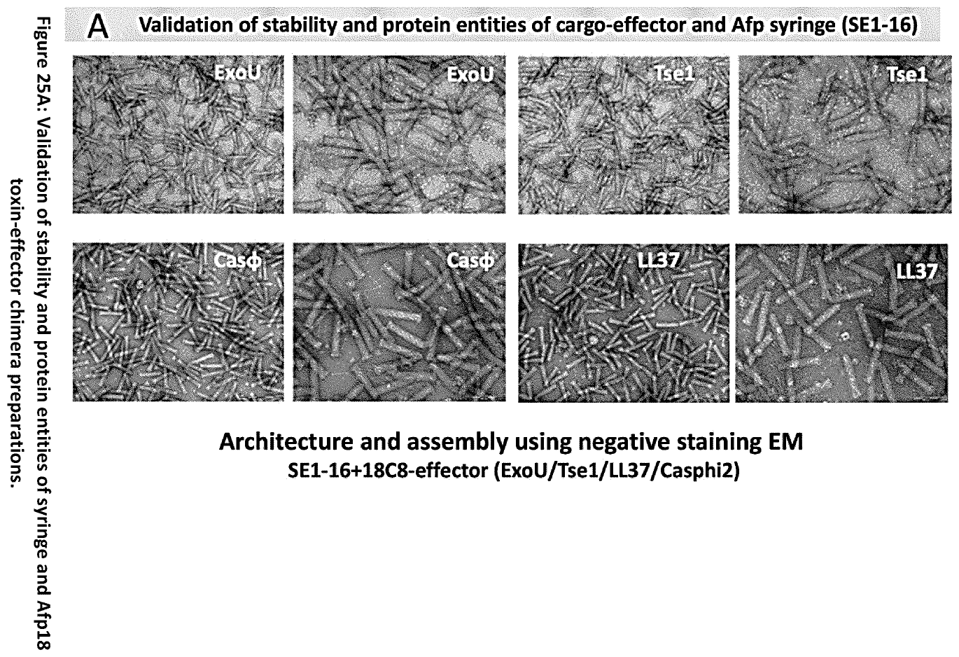

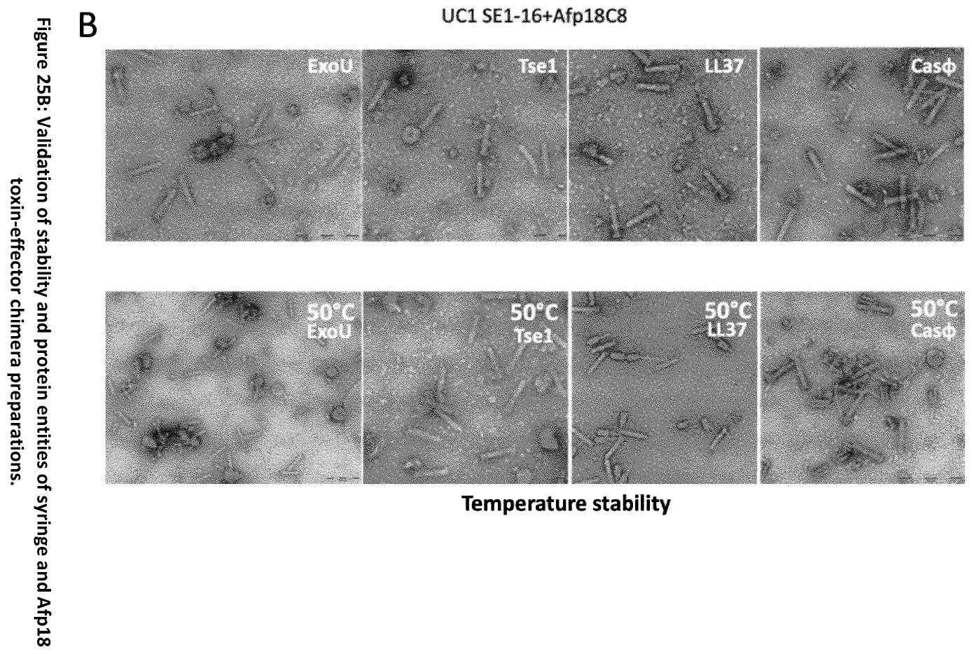

Afp syringe SE1-18 (SEQ. NO. 1) and Afp syringes without toxins SE1-17, SE1-16 (SEQ. NO. 39, 57) were investigated for their effect on moth larve, G. melonella (Lepidoptera, moth). Despite the Afp's native host that is Coleoptera (bugs), killing of Afp syringes including Afp18 toxin could be observed over a period of 13 days FIG. 24 (A). For empty syringes a novel phenotype was observed, that was no moth development, so called arrested state. Modified syringes with anti-eukaryotic effectors, LL37 and ExoU, were investigated for their killing efficacy in vivo in G. melonella larvae. Wild type syringes SE1-18 (SEQ. NO. 1), syringes without toxins SE1-17, SE1-16 (SEQ. NO. 39, 57) and modified syringes including ExoU or LL37 (SEQ. NO. 86 153, 159) were injected and larvae killing observed over time. 100% larvae killing efficacy was observed after 3 days, significantly improved killing when compared to controls (PBS, pBAD33 empty vector) or wild type syringe FIG. 24 (C). Afp larvae ap-peared to be extremely heat stable, a valuable property for field applications FIG. 24 (B). The results are shown in FIG. 24.

Conclusion: The Afp with its Afp18 toxin kills G. melonella larvae after 13 days period. Afp without toxin cargo induces dormancy (no moth formation) after 5 days. When the toxin cargo chimeras are injected, significantly increased larval mortality can be observed already after 3 days for Afp18C8-ExoU and SEAfp18NT20-LL37, the two anti-eukaryotic effectors. The experiments proof that redesigned Afps reveal increased efficacy in vivo.

### Example 11: Qualtity Control of Complete Modified Afp Syringes

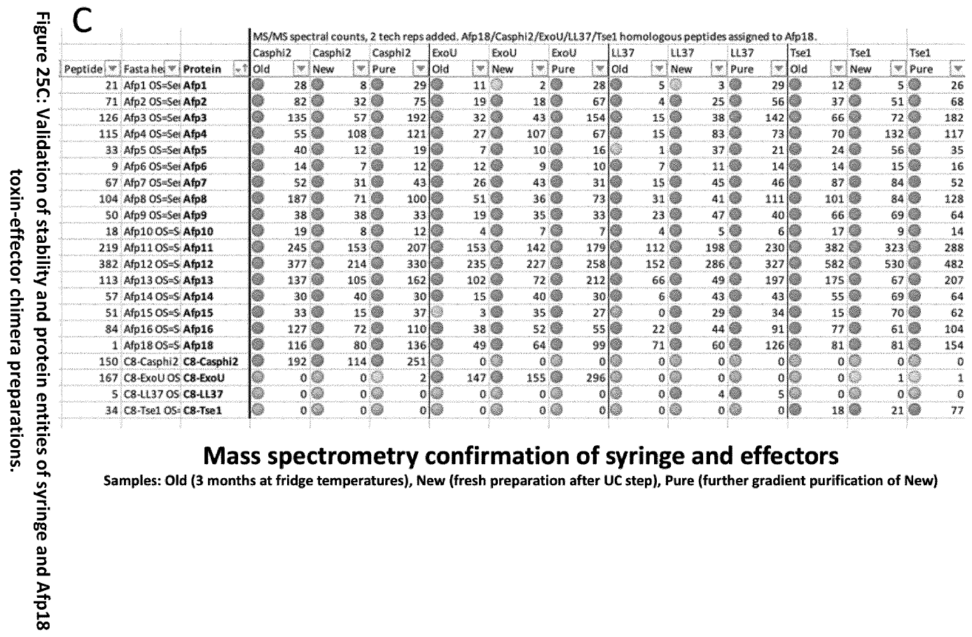

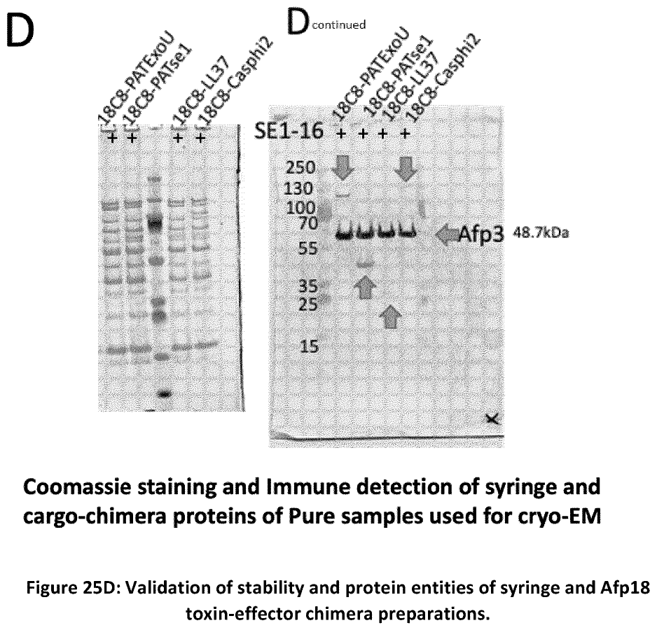

To ensure syringes are of complete architecture and all syringe and effector components have been co-produced, validation experiments are carried out to ensure syringes of high quality so they can be used for cryo-EM analysis or in case a product of pure quality is required. Syringes SE1-16 co-produced with Afp18C8-effectors show complete syringe architecture and similar production levels FIG. 25 (A) Temperature stability of preparations was investigated at 50° C. and analyzed using negative staining electron microscopy and appear stable after high temperature exposure FIG. 25 (B). By using mass spectrometry validation we can confirm all components being present FIG. 25 (D). Further validation of protein and effector components was conducted over coomassie protein gel analysis and immune-detection blotting. The syringe preparations were used for cryo-EM analysis FIG. 23 (B), Cryo-EM analysis of empty and syringes with Afp18 toxin and toxin chimeras)

Conclusion: Afp syringes (SE1-16) co-produced with Afp18-effector chimeras show complete syringe integrity, maintain temperature stability and hold all syringe and effector components.

### REFERENCES

Desfosses et al., 2019, Atomic structures of an entire contractile injection system in both the extended and contracted states, Nature Microbiology, Volume 4, pages 1885-1894.
Eric Duchard, WO2003087377A9 WIPO (PCT)
Grimont et al., 1988, Serratia entomophila sp. nov. Associated with Amber Disease in the New Zealand Grass Grub Costelytra zealandica, International Journal of Systematic Bacteriology, Volume 38, Issue 1, pages 1-6.
Guzman et al., 1995, Tight Regulation, Modulation, and High-Level Expression by Vectors Containing the Arabinose PBAD Promoter, Journal of Bacteriology, July 1995, p. 4121-4130.
Hellberg et al., Peptide Quantitative Structure-Activity Relationships, a Multivariate Approach, Journal of Medicinal Chemistry, 1987, 1126-1135 Hurst et al., Isolation and characterization of the Serratia entomophila antifeeding prophage, FEMS Microbiol. Letter, 2007, 42-48
Jiang et al., 2019, Cryo-EM Structure and Assembly of an Extracellular Contractile Injection System, Cell, Volume 177, Issue 2, pages 370-383.e15.
Pausch et al., 2020, DNA interference states of the hypercompact CRISPR-Casφ effector, Nature Structural Biology, Volume 28, pages 652-661.
Wang et al., 2016, RecET direct cloning and Redap recombineering of biosynthetic gene clusters, large operons or single genes for heterologous expression. Nature Protocols. 2016; 11(7):1175-90.
Xu et al., 2022, Identification and structure of an extracellular contractile injection system from the marine bacterium Algoriphagus machipongonensis, Nature Microbiology, Volume 7, pages 397-410
Jiang et al. 2022, N-terminal signal peptides facilitate the engineering of PVC complex as a potent protein delivery system, Science Advances 8, eabm2343.
Punjani, A., Rubinstein, J. L., Fleet, D. J., Brubaker, M. A., 2017. cryoSPARC: algorithms for rapid unsupervised cryo-EM structure determination. Nat Methods 14, 290-296. https://doi.org/10.1038/nmeth.4169
Punjani, A., Zhang, H., Fleet, D. J., 2020. Non-uniform refinement: adaptive regularization improves single-particle cryo-EM reconstruction. Nat Methods 17, 1214-1221. https://doi.org/10.1038/s41592-020-00990-8
Rohou, A., Grigorieff, N., 2015. CTFFIND4: Fast and accurate defocus estimation from electron micrographs. Journal of Structural Biology 192, 216-221. https://doi.org/10.1016/j.jsb.2015.08.008
Pettersen E F, Goddard T D, Huang C C, Meng E C, Couch G S, Croll T I, Morris J H, Ferrin T E. UCSF ChimeraX: Structure visualization for researchers, educators, and developers. Protein Sci. 2021 January; 30(1):70-82. doi: 10.1002/pro.3943. Epub 2020 Oct. 22. PMID: 32881101; PMCID: PMC7737788.

1. Contractile injection system comprising a Conserved Virulence Cassette controlled by a promoter, said Conserved Virulence cassette comprising structural operons selected from subtypes Ia, Ib, IIa, IIb, IIc, or IId, comprising Open Reading Frames (ORF) encoding a proteinaceous phage tail like syringe structure which functions as a delivery system of cargo, said Conserved Virulence Cassette being co-expressed with an Open Reading Frame controlled by a second promoter encoding a signal peptide and an effector cargo, said signal peptide comprising a packing motif that enables packing of said effector cargo into the syringe, said effector cargo being different from the cargo found in the natural contractile injection system of the Conserved Virulence Cassette and being of up to 300 kDa; wherein said packing motif has a covariance less than zero, said covariance being a function of a first vector and a second vector, said first vector comprising the amino acid hydrophilicities of each amino acid in said packing motif and said second vector comprising the electronic properties of each amino acid in said packing motif, said covariance having a lag of 2 for said first vector and for said second vector.

2. The contractile injection system according to claim 1, wherein the signal peptide comprises at least 20 amino acids.

3. The contractile injection system according to claim 1, wherein the packing motif is positioned N-terminal to effector cargo.

4. Contractile injection system according to claim 1, wherein the Conserved Virulence Cassette is of Class 1 and selected from the group comprising subtypes Ia and Ib derived from Gram-negative phyla from Proteobacteria, Bacteroidetes and Cyanobacteria.

5. Contractile injection system according to claim 4, wherein the Conserved Virulence Cassette is an anti-feeding prophage cassette of subtype Ia with Open Reading Frames afp1 to afp16, from bacteria of the genus Serratia, Salmonella, Erwinia, Yersinia, or Photorhabdus.

6. Contractile injection system according to claim 1, wherein the signal peptide comprising the packing motif consists of 20 amino acids.

7. Contractile injection system according to claim 1, wherein the packing motif comprises more than 60% of polar amino acids selected from lysine, histidine, arginine, glutamic acid, asparagine, serine, threonine, glutamine, and combinations thereof.

8. Contractile injection system according to claim 1, wherein the sequence encoding cargo encodes an antiviral compound, an antibacterial compound, an antifungal compound, an anti-eukaryotic compound, or a gene modification tool.

9. Contractile injection system according to claim 1, wherein the signal peptide is selected from: MPYSSESKEKETHSKETERD (SEQ. NO. 87, 104), MPYFNKSKKNEIRPEKSKEE (SEQ. NO. 218), MPYSRESKEKEIHAKETERD (SEQ. NO. 219), MPYFNELNEKETRSKETESG (SEQ. NO. 220), MLYSSESKEKKTHSKETERD (SEQ. NO. 221), MPYFRESKEKDTHAKESKQD (SEQ. NO. 222), MPYSRESKEKDTHAKGSKQD (SEQ. NO. 223), MPYCNASKQKETYLKEIERD (SEQ. NO. 237), MPYSSESKLKDTHLKEAESD (SEQ. NO. 224), MPYTHSKETERDSSESKEKE (SEQ. NO. 205), MPYDRETEKSHTEKEKSESS (SEQ. NO. 206), MDPRYESTSEEKSSEHETKE (SEQ. NO. 207), MRDPYTESSKEESHSKETKE (SEQ. NO. 208), MPYSSESKEKETHSKE (SEQ. NO. 209), MPYSSESKEKETHESKE, MPYSSESKEKETHS (SEQ. NO. 210), MPYSSESKEKET (SEQ. NO. 211), MLPITAKKTNPFQELEQILA (SEQ. NO. 216), MILPTKAKTFNPQEELQILA (SEQ. NO. 217), MNISSFFLNEENIRFNNQC, MLSTEKHNKDTKHPRNREKK (SEQ. NO. 230), MPNSKYSEKVNHSANGAEKC (SEQ. NO. 231), MPRYSNSQRTPTQSTKNTRR (SEQ. NO. 226), MEHEYSEKEKPQGKKPLIKS (SEQ. NO. 253), MEREYSEKEKHKKHPIQLRD (SEQ. NO. 234), MVHEYSINDRQKRHSFSSAN (SEQ. NO. 235), MLKYANPQTVATQRTKNTA (SEQ. NO. 228), LLTHINLIFRVKCKYSICCLF (SEQ. NO. 239), LCAFPIDGYTNERANQGCGE (SEQ. NO. 240), MNISSYFFLNEENIRFNNQC (SEQ. NO. 225), IIFTRDRNPTLSAHIKGGKK (SEQ. NO. 241), MLSTEKHNKDTKHPRNREKK (SEQ. NO. 230), EVNNGGNKSKAQAHTPDLVM (SEQ. NO. 242), ELKHDDSKIKSQVSIPNLVK (SEQ. NO. 243), TIVNPYSIYLKHIPNGFQDA (SEQ. NO. 244), MEHEYSEKEKPQKCPIQLRD (SEQ. NO. 227), LGDDIMPISNLAKESEVRAV (SEQ. NO. 245), MYDSKKKNSEPTTKKKFERS (SEQ. NO. 232), VGNKNTPSRVKIFISALIFM (SEQ. NO. 246), MPNKKYSENTHQGKKPLIKS (SEQ. NO. 233), MNISSYFFLNEENIKFNNQY (SEQ. NO. 238), MPRYANYQJNPKQNIKNSHG (SEQ. NO. 247), MMFDENLECVNEIINEKLD (SEQ. NO. 248), SENKTKHNQQDSTAKDCWHE (SEQ. NO. 249), ESHSDDKHRDQETNQKTANK (SEQ. NO. 250), EDDSKDHFKQSRNQTEKHYN (SEQ. NO. 251), EDHVNKKKQHTTSSDQDINE (SEQ. NO. 252).

10. Contractile injection system according to claim 5, wherein Open Reading Frames afp1 to afp16 is co-expressed with an Open Reading Frame comprising C-terminal truncated afp18 encoding a signal peptide selected from: MPYSSESKEKETHSKETERD (SEQ. NO. 87, 104), MPYFNKSKKNEIRPEKSKEE (SEQ. NO. 218), MPYSRESKEKEIHAKETERD (SEQ. NO. 219), MPYFNELNEKETRSKETESG (SEQ. NO. 220), MLYSSESKEKKTHSKETERD (SEQ. NO. 221), MPYFRESKEKDTHAKESKQD (SEQ. NO. 222), MPYSRESKEKDTHAKGSKQD (SEQ. NO. 223), MPYCNASKQKETYLKEIERD (SEQ. NO. 237), MPYSSESKLKDTHLKEAESD (SEQ. NO. 224), MPYTHSKETERDSSESKEKE (SEQ. NO. 205), MPYDRETEKSHTEKEKSESS (SEQ. NO. 206), MDPRYESTSEEKSSEHETKE (SEQ. NO. 207), MRDPYTESSKEESHSKETKE (SEQ. NO. 208), MPYSSESKEKETHSKE (SEQ. NO. 209), MPYSSESKEKETHS (SEQ. NO. 210), MPYSSESKEKET (SEQ. NO. 211), MLPITAKKTNPFQELEQILA (SEQ. NO. 216), MILPTKAKTFNPQEELQILA (SEQ. NO. 217), MNISSFFLNEENIRFNNQC, MLSTEKHNKDTKHPRNREKK (SEQ. NO. 230), MPNSKYSEKVNHSANGAEKC (SEQ. NO. 231), MPRYSNSQRTPTQSTKNTRR (SEQ. NO. 226), MEHEYSEKEKPQGKKPLIKS (SEQ. NO. 253), MEREYSEKEKHKKHPIQLRD (SEQ. NO. 234), MVHEYSINDRQKRHSFSSAN (SEQ. NO. 235), MLKYANPQTVATQRTKNTA (SEQ. NO. 228), LLTHINLIFRVKCKYSICCLF (SEQ. NO. 239), LCAFPIDGYTNERANQGCGE (SEQ. NO. 240), MNISSYFFLNEENIRFNNQC (SEQ. NO. 225), IIFTRDRNPTLSAHIKGGKK (SEQ. NO. 241), MLSTEKHNKDTKHPRNREKK (SEQ. NO. 230), EVNNGGNKSKAQAHTPDLVM (SEQ. NO. 242), ELKHDDSKIKSQVSIPNLVK (SEQ. NO. 243), TIVNPYSIYLKHIPNGFQDA (SEQ. NO. 244), MEHEYSEKEKPQKCPIQLRD (SEQ. NO. 227), LGDDIMPISNLAKESEVRAV (SEQ. NO. 245), MYDSKKKNSEPTTKKKFERS (SEQ. NO. 232), VGNKNTPSRVKIFISALIFM (SEQ. NO. 246), MPNKKYSENTHQGKKPLIKS (SEQ. NO. 233), MNISSYFFLNEENIKFNNQY (SEQ. NO. 238), MPRYANYQJNPKQNIKNSHG (SEQ. NO. 247), MMFDENLECVNEIINEKLD (SEQ. NO. 248), SENKTKHNQQDSTAKDCWHE (SEQ. NO. 249), ESHSDDKHRDQETNQKTANK (SEQ. NO. 250), EDDSKDHFKQSRNQTEKHYN (SEQ. NO. 251), EDHVNKKKQHTTSSDQDINE (SEQ. NO. 252).

11. Contractile injection system according to claim 1, wherein the Conserved Virulence Cassette is selected from gene locations: afp1 (AAT48338)-afp18 (AAT48355), afp1 (KHA73_24215)-afp18 (KHA73_24130) from Serratia entomophila, DJ39_RS03165-DJ39_RS03245 from Yersinia ruckeri, L581_RS22600-L581_RS22520 from Serratia fonticola, IFT93_RS22375-IFT93_RS22455 from Erwinia persicina, AEP37_RS09525-AEP37_RS09605 from Yersinia pekkanenii, JFQ86_16445-JFQ86_RS16365 from Serratia ureilytica, C0558_08345-C0558_08275 from Serratia marcescens, JDD69_004072-JDD69_004088 from Salmonella enterica, PluDJC_08620-PluDJC_08545, PluDJC_08725-PluDJC_08650, PluDJC_08815-PluDJC_08740, PluDJC_08925-PluDJC_08845, PluDJC_12605 PluDJC_12675, PluDJC_13295-PluDJC_13220 from Photorhabdus luminescens, and PAU_RS10200-PAU_RS10140, PAU_RS10855-PAU_RS10770, PAU_RS09635-PAU_RS09710, PAU_RS13565-PAU_RS13640, PAU_RS16655-PAU_RS16580 from Photorhabdus asymbiotica.

12. Contractile injection system according to claim 1, wherein the Conserved Virulence Cassette is encoded on the pADAP plasmid with GenBank number CP082788.1.

13. Contractile injection system according to claim 1, wherein said packing motif covariance is a Cross Covariance (CC), said Cross Covariance being calculated by
$${CC}_{za \neq zb , lag} = {\sum }_{i}^{n - lag} \frac{Vza , i \times Vzb , i + lag}{{( n - lag )}^{p}} ,$$
wherein CC is the cross covariances between the first vector Vza comprising amino acid hydrophilicities of each amino acid in said packing motif and the second vector Vzb comprising the electronic properties of each amino acid in said packing motif, where z is the number of z-scales used, i is the position of each amino acid and is a number between 1 and 20, n is the number of amino acids comprised by the vector, I=2 is the lag, p is the normalization degree and V is the descriptor value.

14. Contractile injection system according to claim 1, wherein the first vector comprising the amino acid hydrophilicities of each amino acid in said packing motif and said second vector comprising the electronic properties of each amino acid in said packing motif have the following values:

| | | Second |
|---|---|---|
| Amino acid | First vector | vector |
| Ala (A) | 0.07 | 0.09 |
| Val (V) | −2.69 | −1.29 |
| Leu (L) | −4.19 | −0.98 |
| Ile (I) | −4.44 | −1.03 |
| Pro (P) | −1.22 | 2.23 |
| Phe (F) | −4.92 | 0.45 |
| Trp (W) | −4.75 | 0.85 |
| Met(M) | −2.49 | −0.41 |
| Lys (K) | 2.84 | −3.14 |
| Arg (R) | 2.88 | −3.44 |
| His (H) | 2.41 | 1.11 |
| Gly (G) | 2.23 | 0.30 |
| Ser (S) | 1.96 | 0.57 |
| Thr (T) | 0.92 | −1.40 |
| Cys (C) | 0.71 | 4.13 |
| Tyr (Y) | −1.39 | 0.01 |
| Asn (N) | 3.22 | 0.84 |
| Gln (Q) | 2.18 | −1.14 |
| Asp (D) | 3.64 | 2.36 |
| Glu (E) | 3.08 | −0.07 |

15. A host selected from a eukaryote cell or a prokaryote cell comprising a Conserved Virulence cassette comprising structural operons selected from subtypes Ia, Ib, IIa, IIb, IIc, or IId, comprising Open Reading Frames (ORF) encoding a proteinaceous phage tail like syringe structure which functions as a delivery system of cargo, said Conserved Virulence Cassette being co-expressed with an Open Reading Frame controlled by a second promoter encoding a signal peptide and an effector cargo, said signal peptide comprising a packing motif that enables packing of said effector cargo into the syringe, said effector cargo being different from the cargo found in the natural contractile injection system of the Conserved Virulence Cassette and being of up to 300 kDa; wherein said packing motif has a covariance less than zero, said covariance being a function of a first vector and a second vector, said first vector comprising the amino acid hydrophilicities of each amino acid in said packing motif and said second vector comprising the electronic properties of each amino acid in said packing motif, said covariance having a lag of 2 for said first vector and for said second vector, wherein the Conserved Virulence cassette is expressed in the host.

16. A method for controlling agricultural pests, comprising administering the contractile injection system according to claim 1.

17. Contractile injection system according to claim 1, comprising a medicament or a genetic modification tool as effector cargo for use in delivering a medicament or a genetic modification tool.

18. The method according to claim 16, wherein the agricultural pests are selected from the group consisting of bacteria, nematodes, fungi, and insects at any developmental stage.

19. The host cell according to claim 15, wherein the host cell is selected from the list consisting of yeast, bacteria, E. coli, and the native host.
